(19) United States
(12) Patent Application Publication
(10) Pub. No.: US 2016/0244511 A1
(43) Pub. Date: Aug. 25, 2016

(54) CROSS-REACTIVE STAPHYLOCOCCUS AUREUS ANTIBODY SEQUENCES

(71) Applicant: ARSANIS BIOSCIENCES GMBH, Vienna (AT)

(72) Inventors: Eszter NAGY, Vienna (AT); Adriana BADARAU, Vienna (AT); Harald ROUHA, Wein (AT); Gabor NAGY, Sopron (HU); Irina MIRKINA, Vienna (AT); Zoltan MAGYARICS, Vienna (AT); Zebra VISRAM, Vienna (AT); Michael Benjamin BATTLES, Lebanon, NH (US); Bianka Dominique PRINZ, Lebanon, NH (US); Tushar S. JAIN, Lebanon, NH (US)

(21) Appl. No.: 15/029,944

(22) PCT Filed: Oct. 17, 2014
(86) PCT No.: PCT/EP2014/072316

§ 371 Date: Apr. 15, 2016

(30) Foreign Application Priority Data
Oct. 17, 2013 (EP) 13189095.6

(51) Int. Cl. C07K 16/12; G01N 33/569

(52) U.S. Cl. CPC C07K 16/1271; G01N 33/56938; C07K 2317/565; C07K 2317/515; G01N 2800/26

(57) ABSTRACT

The invention refers to a cross-neutralizing antibody comprising at least one polyspecific binding site that binds to alpha-toxin (Hla) and at least one of the bi-component toxins of Staphylococcus aureus, which antibody comprises at least three complementarity determining regions (CDR1 to CDR3) of the antibody heavy chain variable region (VH), wherein A) the antibody comprises a) a CDR1 comprising or consisting of the amino acid sequence YSISSGMGWG (SEQ ID 1); and b) a CDR2 comprising or consisting of the amino acid sequence SIDQRGSTYYNPSLKS (SEQ ID 2); and c) a CDR3 comprising or consisting of the amino acid sequence ARDAGHGVDMDV (SEQ ID 3); or B) the antibody comprises at least one functionally active CDR variant of a) the parent CDR1 consisting of the amino acid sequence of SEQ ID 1; or b) the parent CDR2 consisting of the amino acid sequence of SEQ ID 2; or c) the parent CDR3 consisting of the amino acid sequence of SEQ ID 3; wherein the functionally active CDR variant comprises at least one point mutation in the parent CDR sequence, and comprises or consists of the amino acid sequence that has at least 60% sequence identity with the parent CDR sequence. It further refers to such cross-neutralizing antibody which is a functionally active variant antibody of a parent antibody that comprises a polyspecific binding site of the VH amino acid sequence of SEQ ID 20, and the VL amino acid sequence of SEQ ID 39, which functionally active variant antibody comprises at least one point mutation in any of the framework regions (FR) or constant domains, or complementarity determining regions (CDR1 to CDR6) in any of SEQ ID 20 or SEQ 39, and has an affinity to bind each of the toxins with a Kd of less than 10⁻⁸M, preferably less than 10⁻⁹M.

The invention refers to cross-neutralizing antibodies comprising at least one polyspecific binding site that binds to alpha-toxin (Hla) and at least one of the bi-component toxins of Staphylococcus aureus, which are characterized by specific amino acid sequences.

### BACKGROUND OF THE INVENTION

Staphylococcus aureus infections represent a significant unmet medical need. S. aureus is one of the most common causes of healthcare associated infections with a particularly high mortality among patients who develop pneumonia, bacteremia and/or sepsis. The spread of antibiotic resistant clones (hospital and community associated methicillin resistant S. aureus, HA- and CA-MRSA) is an additional concern and underscores the need for novel therapeutic approaches.

New antibiotics will unlikely be able to address this medical problem, mostly due to rapidly developing drug resistance and the inability of antibiotics to counteract virulence mechanisms, e.g. the cytolytic effects that contribute to disease progression and mortality in severely infected patients. Several attempts have been made to induce protective immunity either by prophylactic vaccination or passive immunization (i.e. administration of monoclonal antibodies mAbs). These efforts were aimed at enhancing opsonophagocytic uptake and killing by phagocytic cells yet have fallen short of preventing or treating S. aureus infections in a clinical setting.

The recent discovery of the major contribution of exotoxins to the pathogenesis of S. aureus infections has led to new immune prophylactic and therapeutic approaches. Alpha-hemolysin (Hla, or alpha-toxin) was shown to be a major virulence factor that damages epithelial and endothelial cells and has been validated as a vaccine antigen and monoclonal antibody target in animal models of S. aureus disease, reviewed by Beribe and Bubeck Wardenburg (Berube B J, Toxins (Basel). 2013 5(6):1140). More recently Hla has been evaluated in human trials in both active and passive immunization settings.

Wardenburg et al. (The Journal of Experimental Medicine 2008, 205(2): 287-294) describes active immunization with a vaccine based on a mutant form of Hla, which cannot form pores and generates antigen-specific immunoglobulin G responses.

Lin et al. (Expert Review of Clinical Pharmacology 2010, 3(6): 735-767) describes the virulence factors and pathogenesis of staphylococcal infections, and recent developments including vaccines.

Heveker et al. (Human Antibodies and Hybridomas 1994, 5(1-2): 18-24) describes a human monoclonal antibody against staphylococcal alpha-toxin which has been established by hybridoma technology. By such anti-alpha-toxin antibody, none of the bi-component toxins of Staphylococcus aureus is targeted because the bi-component toxins do not contain alpha-toxin and are considered distinct from alpha-toxin.

Ragle et al. (Infection and Immunity 2009, 77(7): 2712-2718) describes anti-alpha-toxin monoclonal antibodies which are able to block the formation of a stable alpha-toxin oligomer. Again, such alpha-toxin antibodies would not target any of the bi-component toxins.

Members of the bi-component cytotoxin family, gamma-hemolysins (HlgAB and HlgCB), Panton Valentine Leukocidin (PVL or LukSF), LukED and LukGH/LukAB can all lyse human phagotyic cells and thus have been implicated in the evasion of innate immunity, a hallmark of S. aureus pathogenesis. In addition, HlgAB is a potent toxin for human red blood cells and LukED has been recently reported to target human T cells via the CXC5 receptor (Alonzo, Nature 2013, 493:51-55). HlgAB and LukED contribute to virulence of S. aureus in murine systemic infection and abscess models, while PVL/LukSF is active only in rabbit models (reviewed by Alonzo PLoS Pathog. 2013, 9(2):e1003143).

The vast majority of S. aureus clinical isolates express Hla, HlgAB and HlgCB, and approximately 40-75% of them LukED. The LukSF/PVL toxin is encoded by phages that are present in 5-10% of strains and implicated in more severe disease manifestation (reviewed by Vandenesch, Front Cell Infect Microbiol. 2012, 2:12).

Contribution of these exotoxins to human S. aureus diseases is implicated based on gene prevalence and sero-epidemiological studies, the latter suggesting a correlation between high serum antibody levels and favorable clinical outcome reported by two independent research groups (Adhikari, J Infect Dis. 2012, 206:915; Fritz, Olin Infect Dis. 2013, 56(11):1554). Therefore, supplementing the human serum antibody repertoire with exotoxin neutralizing monoclonal antibodies is expected to decrease mortality, in particular in patients with low endogenous levels of these toxin neutralizing IgGs.

The subunits of the leukocidins, the S- and F-components—secreted individually in inactive forms—are highly related structurally and share up to 80% amino acid identity. The bi-component toxin subunits and Hla all form barrel-like oligomeric pore complexes upon binding to target cells and have similar structures in spite of low amino acid sequence conservation (<28%).

Gouaux et al. (Protein Science 1997, 6: 2631-2635) describes the differences in sequence and similarity in structure of alpha-toxin, gamma-hemolysin and leukocidin.

The crystal structure of Hla, LukS, LukF, HlgA and HlgB have been determined, and revealed some structural homology, in spite of the low level of amino acid homology between Hla and the bi-component toxin subunits with 16-28% amino acid identity (Galdiero, Protein Sci, 2004:1503; Pedelacq, Structure, 1999:277; Menestrina, FEBS Letters, 2003:54). All these toxins form a ring-like structure formed by oligomerized subunits, leading to pore formation within the cell membrane and subsequent cytolysis. In case of Hla, the pore has been shown to be heptameric, but for the bi-component toxins, hexameric (Comai, Mol Microbial, 2002, 44:1251), heptameric and octameric (Yamashita, PNAS, 2011, 108:17314) heterooligomers have been reported (reviewed in detail by Kaneko, Biosci Biotechnol Biochem, 2004, 68: 981).

The different F- and S-components of this toxin family can form not only cognate pairs (these are: LukS-LukF, LukE-LukD, HlgC-HlgB, HlgA-HlgB and LukH-LukG), but also non-cognate pairs, many of those pairs reported by Gravet et al. (Gravet, FEBS Letters, 1998, 436: 202) and by Dalla Serra et al. for gamma hemolysins and LukS (Dalla Serra, J Chem Inf Model, 2005, 45:1539). Due to the redundancy and promiscuous nature of this toxin family, inactivating one single component is unlikely to be effective to fight S. aureus infections. This notion is supported by observations reported in the literature when neutralization of a single bi-component toxin only partially affected the phenotype (e.g. Ventura, PloS ONE, 2010, 5:e11634; Malachowa, PloS ONE, 2011, 6: e18617). Animal studies showed a differential impact of the various bi-component toxins on the survival, depending on the model employed or the species used for in vivo experiments. The most prominent reduction in disease severity was observed when multiple toxins were deleted, e.g. as in a rabbit model of infection using a knock-out strain of S. aureus where the agr quorum sensing system, a global regulator of toxin expression was inactivated (Kobayashi, J Infect Dis, 2011, 204: 937). Therefore, it is expected that antibody cocktails neutralizing more toxins offer a significant advantage over mAbs against single toxins. However, monoclonal antibody (mAb) cocktails comprising of more than three components are challenging to be developed.

US 2011/274692 A1describes antibodies specific to either LukA, or to LukB, no cross-reactivity of the antibodies is described.

The likelihood of finding single antibodies that cross-react between alpha hemolysin and any of the bi-component toxins was considered to be low, based on the low (<28%) sequence homology between Hla and bi-component toxins. The chance of finding single antibodies cross-reactive among S- and F-components is expected to be higher, due to the higher level of sequence homology (68-82%), with the exception of LukGH that has 30-40% amino acid identity with any S- or F-components. It has been described that hyperimmune serum from animals immunized with LukS can recognize HlgC, however, this is due to the presence of different specificities in the polyclonal serum. Laventie et al. (Laventie, PNAS, 2011, 108:16404) described a bi-specific antibody against LukS that cross-reacts with HlgC. In summary, no cross-reactive mAbs against different bi-component S. aureus toxins or against alpha hemolysin and any of the bi-component toxins have been reported to date.

Given the complex pathogenesis of S. aureus, there is a need to develop an antibody that is able to inactivate several exotoxins, which would significantly increase the potency of anti-S. aureus therapy.

### SUMMARY OF THE INVENTION

It is the objective of the present invention to provide for an antibody directed against the different S. aureus cytotoxins with improved cross-reactive or cross-neutralizing potency. In particular, it is the objective to provide for a monoclonal antibody with nanomolar or sub-nanomolar affinity to at least 2, 3 or 4 different toxin molecules, specifically Hla, HlgB, LukF and LukD. Specifically, the objective refers to an antibody with high neutralizing potency against Hla and multiple bi-component leukocidins in vitro and improved protection compared to single toxin specific antibodies in relevant animal models.

The object is solved by the subject of the present invention.

According to the invention there is provided a cross-neutralizing antibody comprising at least one polyspecific binding site that binds to alpha-toxin (Hla) and at least one of the bi-component toxins of Staphylococcus aureus, which antibody comprises at least three complementarity determining regions (CDR1 to CDR3) of the antibody heavy chain variable region (VH), wherein

A) the antibody comprises
a) a CDR1 comprising or consisting of the amino acid sequence YSISSGMGWG (SEQ ID 1); and
b) a CDR2 comprising or consisting of the amino acid sequence SIDQRGSTYYNPSLKS (SEQ ID 2); and
c) a CDR3 comprising or consisting of the amino acid sequence ARDAGHGVDMDV (SEQ ID 3);

or

B) the antibody comprises at least one functionally active CDR variant of
a) the parent CDR1 consisting of the amino acid sequence of SEQ ID 1; or
b) the parent CDR2 consisting of the amino acid sequence of SEQ ID 2; or
c) the parent CDR3 consisting of the amino acid sequence of SEQ ID 3;

wherein the functionally active CDR variant comprises at least one point mutation in the parent CDR sequence, and comprises or consists of the amino acid sequence that has at least 60% sequence identity with the parent CDR sequence, preferably at least 70%, at least 80%, at least 90% sequence identity.

Specifically, the antibody is not a prior art antibody, such as produced by the host cell deposited under DSM 26748 and the host cell deposited under DSM 26747. Specifically, the antibody of the invention is not antibody #AB-24 produced by a host cell comprising

i) an antibody light chain designated #AB-24-LC which coding sequence is comprised in the host cell deposited under DSM 26748, and

ii) an antibody heavy chain designated #AB-24-HC which coding sequence is comprised in the host cell deposited under DSM 26747.

Yet, the antibody of the invention may be any functional variant of any such prior art antibody, e.g. with a different amino acid sequence in any of the FR and/or any of the CDR sequences. In particular, the antibody of the invention may be any functional variant of the antibody #AB-24, which has the same epitope specificity as the antibody #AB-24.

Specific variants of the antibody designated #AB-24 are specifically included in the subject of the present claims, including, but not limited to, CDR variants, FR variants, murine, chimeric, humanized or human variants, or any antibody domain combination other than a combination composed of the LC and HC of the deposited material, e.g. an antibody comprising the same CDR1-6 or VH/VL combination, yet, with different FR sequences, including e.g. full-length antibodies of various types, Fab, scFv, etc.

According to a specific aspect, the invention provides for an isolated monoclonal antibody that comprises at least one polyspecific binding site that binds to alpha-toxin (Hla) and at least one of the bi-component toxins of Staphylococcus aureus, e.g. that has the same binding specificity as the antibody designated #AB-24, or that cross-competes with the antibody designated #AB-24, which is derived from the antibody designated #AB-24, or a functionally active variant of the antibody designated #AB-24, preferably wherein the antibody designated #AB-24 is characterized by
a) the antibody light chain which coding sequence is contained in the plasmid that has been used to transform a host cell, which transformed host cell is deposited under DSM 26748; and/or
b) the antibody heavy chain which coding sequence is contained in the plasmid that has been used to transform a host cell, which transformed host cell is deposited under DSM 26747.

Specifically, the antibody designated #AB-24 is composed of an antibody light chain comprising the variable region or the LC encoded by the coding sequence of the plasmid comprised in the E. coli host cell deposited under DSM 26748, and an antibody heavy chain comprising the variable region or the HC encoded by the coding sequence of the plasmid comprised in the E. coli host cell deposited under DSM 26747.

Specifically, the functionally active CDR variant comprises at least one of
a) 1, 2, or 3 point mutations in the parent CDR sequence; or
b) 1 or 2 point mutations in any of the four C-terminal or four N-terminal, or four centric amino acid positions of the parent CDR sequence.

Specifically, the functionally active CDR variant is any of
a) a CDR1 sequence selected from the group consisting of YPISSGMGWG (SEQ ID 4), and YSISSGMGWD (SEQ ID 5); or
b) a CDR2 sequence selected from the group consisting of SVDQRGSTYYNPSLKS (SEQ ID 6), RIDQRGSTYYNPSLKS (SEQ ID 7), RVDQRGSTYYNPSLKS (SEQ ID 8), SIDQRGSTYYNPSLEG (SEQ ID 9), and SIDQRGSTYYNPPLES (SEQ ID 10); or
c) a CDR3 sequence selected from the group consisting of ARDAGHGADMDV (SEQ ID 11), and ARDAGHAVDMDV (SEQ ID 12).

Specifically, the antibody is characterized by the CDR sequences, wherein
a) in VH CDR1 at position 5, the amino acid residue is selected from the group consisting of S, A, D, E, F, G, H, I, K, L, M, N, Q, R, T V, W and Y, preferentially any of H, R and W;
b) in VH CDR1 at position 7, the amino acid residue is selected from the group consisting of M, H, K, Q, R and W, preferentially any of K, R or W;
c) in VH CDR2 at position 3, the amino acid residue is selected from the group consisting of D and R;
d) in VH CDR2 at position 7, the amino acid residue is selected from the group consisting of S, A, D, E, F, H, K, M, N, Q, R, T, W and Y, preferentially any of D, H, K, N or Q, and more preferentially is Q;
e) in VH CDR2 at position 9, the amino acid residue is selected from the group consisting of Y, F, K, L, Q and R, and preferentially is R;
f) in VH CDR3 at position 5, the amino acid residue is selected from the group consisting of G, A, D, F, H, I, M, N, R, S, T, V and Y, preferentially any of D, F, H, I, M, N, R, T, V or Y;
g) in VH CDR3 at position 6, the amino acid residue is selected from the group consisting of H, E, Q and S, preferentially any of E or Q;
h) in VH CDR3 at position 7, the amino acid residue is selected from the group consisting of G, A, D, E, H, I, M, N, Q, S, T, V and W, and preferentially is W; and/or
i) in VH CDR3 at position 8, the amino acid residue is selected from the group consisting of V, A, D, E, G, I, K, L, M, Q, R, S and T, preferentially any of M or R.

According to a specific embodiment, the antibody is selected from the group consisting of

a) an antibody comprising
a. the CDR1 sequence of SEQ ID 1; and
b. the CDR2 sequence of SEQ ID 6; and
c. the CDR3 sequence of SEQ ID 11;

b) an antibody comprising
a. the CDR1 sequence of SEQ ID 4; and
b. the CDR2 sequence of SEQ ID 7; and
c. the CDR3 sequence of SEQ ID 3;

c) an antibody comprising
a. the CDR1 sequence of SEQ ID 1; and
b. the CDR2 sequence of SEQ ID 8; and
c. the CDR3 sequence of SEQ ID 3;

d) an antibody comprising
a. the CDR1 sequence of SEQ ID 1; and
b. the CDR2 sequence of SEQ ID 2; and
c. the CDR3 sequence of SEQ ID 12;

e) an antibody comprising
a. the CDR1 sequence of SEQ ID 5; and
b. the CDR2 sequence of SEQ ID 9; and
c. the CDR3 sequence of SEQ ID 3;

f) an antibody comprising
a. the CDR1 sequence of SEQ ID 5; and
b. the CDR2 sequence of SEQ ID 10; and
c. the CDR3 sequence of SEQ ID 3;

According to a specific aspect, the antibody comprises a VH amino acid sequence selected from the group consisting of SEQ ID 20-31.

According to a specific aspect, the antibody comprises an antibody heavy chain (HC) amino acid sequence selected from the group consisting of SEQ ID 40-51, or any of the amino acid sequences of SEQ ID 40-51 with a deletion of the C-terminal amino acid, or in particular with a deletion of a C-terminal Lysine.

Specifically, SEQ ID 40-51 show the HC sequences which is N-terminally extended by a signal sequence. It is understood that the specific antibody comprises such HC amino acid sequence with or without the respective signal sequence, or with alternative signal or leader sequences.

Specifically, SEQ ID 40 shows the HC sequence which includes the VH amino acid sequence of SEQ ID 20, and which is N-terminally extended by a signal sequence. It is understood that the specific antibody comprises such VH or HC amino acid sequence with or without the respective signal sequence, or with alternative signal or leader sequences.

Specifically, SEQ ID 41 shows the HC sequence which includes the VH amino acid sequence of SEQ ID 21, and which is N-terminally extended by a signal sequence. It is understood that the specific antibody comprises such VH or HC amino acid sequence with or without the respective signal sequence, or with alternative signal or leader sequences.

Specifically, SEQ ID 42 shows the HC sequence which includes the VH amino acid sequence of SEQ ID 22, and which is N-terminally extended by a signal sequence. It is understood that the specific antibody comprises such VH or HC amino acid sequence with or without the respective signal sequence, or with alternative signal or leader sequences.

Specifically, SEQ ID 43 shows the HC sequence which includes the VH amino acid sequence of SEQ ID 23, and which is N-terminally extended by a signal sequence. It is understood that the specific antibody comprises such VH or HC amino acid sequence with or without the respective signal sequence, or with alternative signal or leader sequences.

Specifically, SEQ ID 44 shows the HC sequence which includes the VH amino acid sequence of SEQ ID 24, and which is N-terminally extended by a signal sequence. It is understood that the specific antibody comprises such VH or HC amino acid sequence with or without the respective signal sequence, or with alternative signal or leader sequences.

Specifically, SEQ ID 45 shows the HC sequence which includes the VH amino acid sequence of SEQ ID 25, and which is N-terminally extended by a signal sequence. It is understood that the specific antibody comprises such VH or HC amino acid sequence with or without the respective signal sequence, or with alternative signal or leader sequences.

Specifically, SEQ ID 46 shows the HC sequence which includes the VH amino acid sequence of SEQ ID 26, and which is N-terminally extended by a signal sequence. It is understood that the specific antibody comprises such VH or HC amino acid sequence with or without the respective signal sequence, or with alternative signal or leader sequences.

Specifically, SEQ ID 47 shows the HC sequence which includes the VH amino acid sequence of SEQ ID 27, and which is N-terminally extended by a signal sequence. It is understood that the specific antibody comprises such VH or HC amino acid sequence with or without the respective signal sequence, or with alternative signal or leader sequences.

Specifically, SEQ ID 48 shows the HC sequence which includes the VH amino acid sequence of SEQ ID 28, and which is N-terminally extended by a signal sequence. It is understood that the specific antibody comprises such VH or HC amino acid sequence with or without the respective signal sequence, or with alternative signal or leader sequences.

Specifically, SEQ ID 49 shows the HC sequence which includes the VH amino acid sequence of SEQ ID 29, and which is N-terminally extended by a signal sequence. It is understood that the specific antibody comprises such VH or HC amino acid sequence with or without the respective signal sequence, or with alternative signal or leader sequences.

Specifically, SEQ ID 50 shows the HC sequence which includes the VH amino acid sequence of SEQ ID 30, and which is N-terminally extended by a signal sequence. It is understood that the specific antibody comprises such VH or HC amino acid sequence with or without the respective signal sequence, or with alternative signal or leader sequences.

Specifically, SEQ ID 51 shows the HC sequence which includes the VH amino acid sequence of SEQ ID 31, and which is N-terminally extended by a signal sequence. It is understood that the specific antibody comprises such VH or HC amino acid sequence with or without the respective signal sequence, or with alternative signal or leader sequences.

According to a specific aspect, each of the HC sequences may be terminally extended or deleted in the constant region, e.g. a deletion of one or more or the C-terminal amino acids.

Specifically, each of the HC sequences that comprises an C-terminal Lysine residue is preferably employed with a deletion of such C-terminal Lysine residue.

According to a specific embodiment, the antibody further comprises at least three complementarity determining regions (CDR4 to CDR6) of the antibody light chain variable region (VL), preferably wherein

A) the antibody comprises
a) a CDR4 comprising or consisting of the amino acid sequence RASQGISRWLA (SEQ ID 32); and
b) a CDR5 comprising or consisting of the amino acid sequence AASSLQS (SEQ ID 33); and
c) a CDR6 comprising or consisting of the amino acid sequence QQGYVFPLT (SEQ ID 34);

or

B) the antibody comprises at least one functionally active CDR variant of
a) the parent CDR4 consisting of the amino acid sequence of SEQ ID 32; or
b) the parent CDR5 consisting of the amino acid sequence of SEQ ID 33; or
c) the parent CDR6 consisting of the amino acid sequence of SEQ ID 34;

wherein the functionally active CDR variant comprises at least one point mutation in the parent CDR sequence, and comprises or consists of the amino acid sequence that has at least 60% sequence identity with the parent CDR sequence, preferably at least 70%, at least 80%, at least 90% sequence identity.

Specifically, the antibody is characterized by the CDR sequences, wherein
a) in VL CDR4 at position 7, the amino acid residue is selected from the group consisting of S, A, E, F, G, K, L, M, N, Q, R, W and Y, preferentially any of L, M, R or W, and more preferentially is R;
b) in VL CDR5 at position 1, the amino acid residue is selected from the group consisting of A and G;
c) in VL CDR5 at position 3, the amino acid residue is selected from the group consisting of S, A, D, G, H, I, K, L, N, Q, R, T, V and W;
d) in VL CDR5 at position 4, the amino acid residue is selected from the group consisting of S, D, E, H, I, K, M, N, Q, R, T and V, preferentially any of K, N, Q and R;
e) in VL CDR6 at position 3, the amino acid residue is selected from the group consisting of G, A, D, E, F, H, I, K, L, N, Q, R, S, T, V, W and Y;
f) in VL CDR6 at position 4, the amino acid residue is selected from the group consisting of Y, D, F, H, M, R and W;
g) in VL CDR6 at position 5, the amino acid residue is selected from the group consisting of V, A, D, E, F, G, H, I, K, L, M, N, Q, R, S, T, and W; and/or
h) in VL CDR6 at position 6, the amino acid residue is selected from the group consisting of F and W.

According to a specific aspect, the antibody of the invention comprises CDR combinations as listed in FIG. 1, provided, that the antibody is still functionally active.

Specifically, the antibody of the invention comprises the CDR1-6 of any of the antibodies as listed in FIG. 1. However, according to an alternative embodiment, the antibody may comprise different CDR combinations, e.g. wherein an antibody as listed in FIG. 1 comprises at least one CDR sequence, such as 1, 2, 3, 4, 5, or 6 CDR sequences of one antibody and at least one further CDR sequence of a different antibody of any of the antibodies as listed in FIG. 1. According to a specific example, the antibody comprises 1, 2, 3, 4, 5, or 6 CDR sequences, wherein the CDR sequences are CDR combinations of more than 1 antibody, e.g. 2, 3, 4, 5, or 6 different antibodies. For example, the CDR sequences may be combined to preferably comprise 1, 2, or all 3 of CDR1-3 of any of the antibodies as listed in FIG. 1, and 1, 2, or all 3 of CDR4-6 of the same or any other antibody listed in FIG. 1.

It is herein specifically understood that the CDR5 numbered CDR1, 2, and 3 represent the binding region of the VH domain, and CDR4, 5, and 6 represent the binding region of the VL domain.

According to a specific aspect, the antibody of the invention comprises any of the HC and LC amino acid sequence combinations as depicted in FIG. 1, or the binding site formed by such combination of HC and LC amino acid sequences. Alternatively, combinations of the immunoglobulin chains of two different antibodies may be used, provided, that the antibody is still functionally active. For example, the HC sequence of one antibody may be combined with an LC sequence of another antibody. According to further specific embodiments, any of the framework regions as provided in FIG. 1 may be employed as a framework to any of the CDR sequences and/or VH/VL combinations as described herein.

It is understood that the antibody of the invention optionally comprises such amino acid sequences of FIG. 1, with or without the respective signal sequence, or with alternative signal or leader sequences.

According to a specific aspect, each of the sequences of FIG. 1 may be terminally extended or deleted in the constant region, e.g. a deletion of one or more or the C-terminal amino acids.

FIG. 1 shows 12 different HC sequences with similarities in any of the CDR1, 2, and/or 3, and one LC sequence, and supports any HC/LC combination, wherein one of the CDR1-3 of one HC, e.g. CDR1 is combined with any other CDR sequence of a second and optionally a third HC, e.g. CDR2 and CDR3 of a second and a third HC, respectively.

According to a specific aspect, the antibody comprises a VL amino acid sequence of SEQ ID 39 or an antibody light chain (LC) amino acid of SEQ ID 52.

Specifically, SEQ ID 52 shows the LC sequences which includes the VL amino acid sequence of SEQ ID 39, and which is N-terminally extended by a signal sequence. It is understood that the specific antibody comprises such VL or LC amino acid sequence with or without the respective signal sequence, or with alternative signal or leader sequences.

According to a specific aspect, the antibody of the invention comprises any of the VH amino acid sequences of SEQ ID 20-31, and the VL amino acid sequence of SEQ ID 39, or the binding site formed by such combination of VH and VL amino acid sequences.

According to a specific aspect, the antibody of the invention comprises any of the HC amino acid sequences of SEQ ID 40-51, and the LC amino acid sequence of SEQ ID 52, or the binding site formed by such combination of HC and LC amino acid sequences.

According to the invention there is further provided a cross-neutralizing antibody comprising at least one polyspecific binding site that binds to alpha-toxin (Hla) and at least one of the bi-component toxins of Staphylococcus aureus, which antibody is a functionally active variant antibody of a parent antibody that comprises a polyspecific binding site of the VH amino acid sequence of SEQ ID 20, and the VL amino acid sequence of SEQ ID 39, which functionally active variant antibody comprises at least one point mutation in any of the framework regions (FR) or constant domains, or complementarity determining regions (CDR1 to CDR6) in any of SEQ ID 20 or SEQ 39, and has an affinity to bind each of the toxins with a Kd of less than 10⁻⁸M, preferably less than 10⁻⁹M, preferably less than 10⁻¹⁰M, preferably less than 10⁻¹¹M, e.g. with an affinity in the picomolar range.

Specifically, the functionally active variant antibody comprises at least one of the functionally active CDR variants of the invention.

Specifically, the functionally active variant differs from a parent antibody, e.g. any of the antibodies as listed in FIG. 1, in at least one point mutation in the amino acid sequence, preferably in the CDR, wherein the number of point mutations in each of the CDR amino acid sequences is either 0, 1, 2 or 3.

Specifically, the antibody is derived from such antibodies, employing the respective CDR sequences, or CDR mutants, including functionally active CDR variants, e.g. with 1, 2 or 3 point mutations within one CDR loop, e.g. within a CDR length of 5-18 amino acids, e.g. within a CDR region of 5-15 amino acids or 5-10 amino acids. Alternatively, there may be 1 to 2 point mutations within one CDR loop, e.g. within a CDR length of less than 5 amino acids, to provide for an antibody comprising a functionally active CDR variant. Specific CDR sequences might be short, e.g. the CDR2 or CDR5 sequences. According to a specific embodiment, the functionally active CDR variant comprises 1 or 2 point mutations in any CDR sequence consisting of less than 4 or 5 amino acids.

According to a specific aspect, the antibody of the invention comprises CDR and framework sequences, wherein at least one of the CDR and framework sequences includes human, humanized, chimeric, murine or affinity matured sequences, preferably wherein the framework sequences are of an IgG antibody, e.g. of an IgG1, IgG2, IgG3, or IgG4 subtype, or of an IgA1, IgA2, IgD, IgE, or IgM antibody.

Specific antibodies are provided as framework mutated antibodies, e.g. to improve manufacturability or tolerability of a parent antibody, e.g. to provide an improved (mutated) antibody which has a low immunogenic potential, such as humanized antibodies with mutations in any of the CDR sequences and/or framework sequences as compared to a parent antibody.

Further specific antibodies are provided as CDR mutated antibodies, e.g. to improve the affinity of an antibody and/or to target the same epitope or epitopes near the epitope that is targeted by a parent antibody (epitope shift).

Accordingly, any of the antibodies as listed in FIG. 1 may be used as parent antibodies to engineer improved versions.

Specifically, the functionally active variant antibody has a specificity to bind the same epitope as the parent antibody.

According to a specific aspect, the at least one point mutation is any of an amino acid substitution, deletion and/or insertion of one or more amino acids.

Specifically, the at least one point mutation is any of the amino acid substitutions
S51R or S51K in the CDR2; or
G103A, V104A or V104S in the CDR3.

Specifically the bi-component toxin targeted by the antibody of the invention is selected from the group consisting of cognate and non-cognate pairs of F and S components of gamma-hemolysins (HlgABC), PVL (LukSF) and PVL-like toxins, preferably any of HlgAB, HlgCB, LukSF, LukED, LukGH, LukS-HlgB, LukSD, HlgA-LukD, HlgA-LukF, LukG-HlgA, LukEF, LukE-HlgB, HlgC-LukD or HlgC-LukF.

According to a specific aspect, the antibody has a cross-specificity to bind Hla and at least one of the F-components of the bi-component toxins or the toxins comprising such F-components, preferably at least two or three thereof, preferably Hla and at least three of the F-components of the bi-component toxins, or the toxins comprising such F-components.

Specifically, the F-components are selected from the group consisting of HlgB, LukF and LukD, or any F-component of the cognate and non-cognate pairs of F and S components of gamma-hemolysins, PVL toxins and PVL-like toxins, preferably HlgAB, HlgCB, LukSF, LukED, LukS-HlgB, LukSD, HlgA-LukD, HlgA-LukF, LukEF, LukE-HlgB, HlgC-LukD or HlgC-LukF.

Specifically, the F-component targeted by the antibody of the invention is any one, two or three of HlgB, LukF and LukD.

Preferably the binding site binds to at least two or at least three bi-component toxins, preferably at least two or three of any of HlgAB, HlgCB, LukSF and LukED, preferably all of HlgAB, HlgCB, LukSF and LukED.

According to a specific aspect, the antibody inhibits the binding of one or more of the toxins to phosphocholine or phosphatidylcholine, in particular the phosphatidylcholine of mammalian cell membranes.

According to a specific aspect, the antibody exhibits in vitro neutralization potency in a cell-based assay with an IC50 of less than 100:1 mAb:toxin ratio (mol/mol), preferably less than 50:1, preferably less than 25:1, preferably less than 10:1, more preferably less than 1:1.

According to a further specific aspect, the antibody neutralizes the targeted toxins in animals, including both, human and non-human animals, and inhibits S. aureus pathogenesis in vivo, preferably any models of pneumonia, bacteremia, sepsis, abscess, skin infection, peritonitis, catheter and prosthetic devices related infection and osteomyelitis.

According to a specific aspect, the antibody is a full-length monoclonal antibody, an antibody fragment thereof comprising at least one antibody domain incorporating the binding site, or a fusion protein comprising at least one antibody domain incorporating the binding site. Preferably, the antibody is selected from the group consisting of murine, chimeric, humanized or human antibodies, heavy-chain antibodies, Fab, Fd, scFv and single-domain antibodies like VH, VHH or VL, preferably a human IgG1 antibody.

The invention further provides for an expression cassette or a plasmid comprising a coding sequence to express a light chain and/or heavy chain of an antibody of the invention.

The invention specifically provides for an expression cassette or a plasmid comprising a coding sequence to express

a) a VH and/or VL of an antibody of the invention; or

b) or a HC and/or LC of an antibody of the invention.

The invention further provides for a host cell comprising the expression cassette or the plasmid of the invention.

Specifically, a plasmid and a host cell are excluded, which material is deposited under DSM 26747 or DSM 26748. Such deposits are E. coli host cells transformed with a plasmid, wherein the host cell deposited under DSM 26748 is transformed with the plasmid comprising a nucleotide sequence encoding the antibody light chain designated #AB-24-LC; and the host cell deposited under DSM 26747 is transformed with the plasmid comprising a nucleotide sequence encoding the antibody heavy chain designated #AB-24-HC.

Specifically preferred is a host cell and a production method employing such host cell, which host cell comprises
the plasmid or expression cassette of the invention, which incorporates a coding sequence to express the antibody light chain; and
the plasmid or expression cassette of the invention, which incorporates a coding sequence to express the antibody heavy chain.

The invention further provides for a method of producing an antibody according to the invention, wherein a host cell of the invention is cultivated or maintained under conditions to produce said antibody.

The invention further provides for a method of producing functionally active antibody variants of a parent antibody which is any of the antibodies of the invention, e.g. an antibody as listed in FIG. 1, or comprising any of the HC and LC amino acid sequence combinations as depicted in FIG. 1, or comprising the binding site formed by such combination of HC and LC amino acid sequences, or comprising a polyspecific binding site of the VH amino acid sequence of any of SEQ ID 20-31, and the VL amino acid sequence of SEQ ID 39, which method comprises engineering at least one point mutation in any of the framework regions (FR) or constant domains, or complementarity determining regions (CDR1 to CDR6) in any of SEQ ID 20-31 or SEQ 39 to obtain a variant antibody, and determining the functional activity of the variant antibody by any of
the affinity to bind each of Hla and at least one of the bi-component toxins of S. aureus with a Kd of less than 10⁻⁸M, preferably less than 10⁻⁹M, preferably less than 10⁻¹⁰M, preferably less than 10⁻¹¹M, e.g. with an affinity in the picomolar range, and/or
the binding of the variant antibody to Hla or the at least one of the bi-component toxins in competition with the parent antibody;

wherein upon determining the functional activity, the functionally active variants are selected for production by a recombinant production method.

Functionally active variant antibodies may differ in any of the VH or VL sequences, or share the common VH and VL sequences, and comprise modifications in the respective FR. The variant antibody derived from the parent antibody by mutagenesis may be produced a methods well-known in the art.

Exemplary parent antibodies are described in the examples section below and in FIG. 1. Specifically, the antibody is a functionally active derivative of a parent antibody as listed in FIG. 1. Variants with one or more modified CDR sequences, and/or with one or more modified FR sequences, such as sequences of FR1, FR2, FR3 or FR4, or a modified constant domain sequence may be engineered.

The invention further provides for a method of producing an antibody of the invention, comprising

(a) immunizing a non-human animal with the three-dimensional structure of the epitope as defined herein;

(b) forming immortalized cell lines from the isolated B-cells;

(c) screening the cell lines obtained in b) to identify a cell line producing a monoclonal antibody that binds to the epitope; and

(d) producing the monoclonal antibody, or a humanized or human form of the antibody, or a derivative thereof with the same epitope binding specificity as the monoclonal antibody.

According to a further aspect, the invention provides for a method of producing an antibody of the invention, comprising

(a) immunizing a non-human animal with alpha-toxin and/or at least one of a bi-component toxin of Staphylococcus aureus and isolating B-cells producing antibodies;

(b) forming immortalized cell lines from the isolated B-cells;

(c) screening the cell lines to identify a cell line producing a monoclonal antibody that binds to alpha-toxin and at least one of a bi-component toxin of Staphylococcus aureus; and

(d) producing the monoclonal antibody, or a humanized or human form of the antibody, or a derivative thereof with the same epitope binding specificity as the monoclonal antibody.

The invention further provides for the antibody of the invention for medical use, in particular for use in treating a subject at risk of or suffering from a S. aureus infection comprising administering to the subject an effective amount of the antibody to limit the infection in the subject, to ameliorate a disease condition resulting from said infection or to inhibit S. aureus disease pathogenesis, such as pneumonia, sepsis, bacteremia, wound infection, abscesses, surgical site infection, endothalmitis, furunculosis, carbunculosis, endocarditis, peritonitis, osteomyelitis or joint infection.

Specifically the antibody is provided for protecting against S. aureus infections.

Therefore, the invention provides for a method of treatment, wherein a subject at risk of or suffering from a S. aureus infection is treated by administering to the subject an effective amount of the antibody to limit the infection in the subject, to ameliorate a disease condition resulting from said infection or to inhibit S. aureus disease pathogenesis, such as pneumonia, sepsis, bacteremia, wound infection, abscesses, surgical site infection, endothalmitis, furunculosis, carbunculosis, endocarditis, peritonitis, osteomyelitis or joint infection.

Specifically, the method of treatment is provided for protecting against pathogenic S. aureus.

The invention further provides for a pharmaceutical preparation comprising the antibody of the invention, preferably comprising a parenteral or mucosal formulation, optionally containing a pharmaceutically acceptable carrier or excipient.

Such pharmaceutical composition may contain the antibody as the sole active substance, or in combination with other active substances, or a cocktail of active substances, such as a combination or cocktail of at least two or three different antibodies, e.g. wherein the other active substances are further targeting S. aureus, e.g. an OPK antibody or an antibody targeting at least one other toxin. Specifically, the cocktail of antibodies comprises one or more antibodies of the invention, each targeting toxins and the combination targeting more different epitopes or toxins than only one antibody in the cocktail.

The invention further provides for the antibody of the invention for diagnostic use to detect any S. aureus infections, including high toxin producing MRSA infections, such as necrotizing pneumonia, and toxin production in furunculosis and carbunculosis.

The invention further provides for a diagnostic preparation of the antibody of the invention, optionally containing the antibody with a label and/or a further diagnostic reagent with a label.

Specifically, the antibody is provided for diagnostic use according to the invention, wherein a systemic infection with S. aureus in a subject is determined ex vivo by contacting a sample of body fluid of said subject with the antibody, wherein a specific immune reaction of the antibody determines the infection.

Therefore, the invention further refers to the respective method of diagnosing an S. aureus infection in a subject, in particular wherein a systemic infection with S. aureus in a subject is determined.

The invention further provides for a crystal formed by a Hla monomer that diffracts x-ray radiation to produce a diffraction pattern representing the three-dimensional structure of the Hla rim domain in contact with the antibody of the invention, or a binding fragment thereof, preferably a Fab fragment, having the following cell constants: 285.05 Å, 150.94 Å, 115.25 Å, space group P2₁2₁2, optionally with a deviation of between 0.00 Å and 2.00 Å.

The invention further provides for a crystal formed by a LukD monomer that diffracts x-ray radiation to produce a diffraction pattern representing the three-dimensional structure of the LukD rim domain in contact with the antibody of the invention, or a binding fragment thereof, preferably a Fab fragment, having the following cell constants: 112.0 Å, 112.0 Å, 409.3 Å, space group H32, optionally with a deviation of between 0.00 Å and 2.00 Δ.

The invention further provides for the isolated paratope of an antibody of the invention, or a binding molecule comprising said paratope.

The invention further provides for the isolated conformational epitope recognized by the antibody of the invention, characterized by a three-dimensional structure of the rim domain of Hla, LukD, LukF or HlgB. Such epitope may consist of a single epitope or a mixture of epitopes comprising epitope variants, each recognized by the antibody of the invention.

The invention further provides for the epitope of the invention, characterized by a three-dimensional structure selected from the group consisting of
a) the three-dimensional Hla structure characterized by the structure coordinates of the contact amino acid residues 179-191, 194, 200, 269 and 271 of SEQ ID 54;
b) the three-dimensional LukF structure characterized by the structure coordinates of the contact amino acid residues 176-188, 191, 197 and 267 of SEQ ID 55, preferably with amino acid residues 176-179, 181-184, 186-188, 191, 197 and 267 of SE ID 58;
c) the three-dimensional LukD structure characterized by the structure coordinates of the contact amino acid residues 176-188, 191, 197 and 267 of SEQ ID 54, preferably with amino acid residues 176-179, 181-184, 186-188, 191, 197 and 267 of SEQ ID 62;
d) the three-dimensional HlgB structure characterized by the structure coordinates of the amino acid contact residues 177-189, 192, 198 and 268 of SEQ ID 56, preferably with amino acid residues 177-180, 182-185, 187-189, 192, 198 and 268 of SEQ ID 68,
e) the three-dimensional Hla rim domain structure of the crystal of the invention;
f) the three-dimensional LukD rim domain structure of the crystal of the invention; and
g) a three-dimensional structure which is a homolog of any of a) to f) wherein said homolog comprises a binding site that has a root mean square deviation from backbone atoms of contact amino acid residues of between 0.00 Å and 2.00 Δ.

Specifically, the epitope is bound by a binding molecule.

The invention further provides for a binder or binding molecule which specifically binds to the epitope of the invention, preferably selected from the group consisting of a protein, a peptide, a peptidomimetic, a nucleic acid, a carbohydrate, a lipid, an oligopeptide, an aptamer and a small molecule compound, preferably an antibody, an antibody fragment thereof comprising at least one antibody domain incorporating the binding site, or a fusion protein comprising at least one antibody domain incorporating the binding site.

Specifically, the binder or binding molecule is a polyspecific binder that binds to Hla and at least one of the bi-component toxins of S. aureus.

Specifically, the binder or binding molecule prevents toxin binding to phosphocholine and competes with the antibody of the invention.

The invention further provides for a screening method or assay for identifying a binder which specifically binds to or recognizes the epitope of the invention, comprising the steps of:
bringing a candidate compound into contact with the three-dimensional structure of the epitope as defined herein; and
assessing binding between the candidate compound and the three-dimensional structure; wherein binding between the candidate compound and the three-dimensional structure identifies the candidate compound as a polyspecific binder that binds to Hla and at least one of the bi-component toxins of S. aureus. For example, a positive functional binding reaction between the candidate compound and the three-dimensional structure or the epitope identifies the compound as protective binder.

According to a further aspect, the invention provides for an immunogen comprising:

a) an epitope of the invention;

b) optionally further epitopes not natively associated with said epitope of (a);

and

c) a carrier.

Specifically, the carrier is a pharmaceutically acceptable carrier, preferably comprising buffer and/or adjuvant substances.

The immunogen of the invention is preferably provided in a vaccine formulation, preferably for parenteral use.

Specifically the immunogen of the invention is provided for medical use, specifically for use in treating a subject by administering an effective amount of said immunogen to protect the subject from an S. aureus infection, to prevent a disease condition resulting from said infection or to inhibit S. aureus pneumonia pathogenesis.

Specifically the immunogen of the invention is provided for eliciting a protective immune response.

According to a specific aspect, there is further provided a method of treatment wherein a subject at risk of a S. aureus infection is treated, which method comprises administering to the subject an effective amount of the immunogen to prevent infection in the subject, in particular to protect against pathogenic S. aureus.

According to a further aspect, the invention provides for an isolated nucleic acid encoding an antibody of the invention, or encoding an epitope of the invention.

### FIGURES

FIG. 1: Amino acid sequences and Fab K_{D} affinities of Hla—bi-component toxin cross-reactive mAbs.

Heavy and light chain CDR sequences, FR sequences and full-length sequence information which is the composite sequence of the respective FR and CDR sequences (SEQ ID 1-39), are shown, amino acid changes relative to the parental AB-28 mAb indicated by bold and underlined fonts. Fab K_{D} affinities were measured by MSD method using a Sector Immager 2400 instrument (Meso Scale Discovery). Typically 20 pM of biotinylated antigen was incubated with Fab at various concentrations, for 16 h at room temperature, and the unbound antigen captured on immobilized IgG. See also for example, Estep et al., “High throughput solution-based measurement of antibody-antigen affinity and epitope binning”, MAbs, Vol. 5(2), pp. 270-278 (2013). Fab K_{D} affinities are indicated in pM for each antibody and for each toxin components.

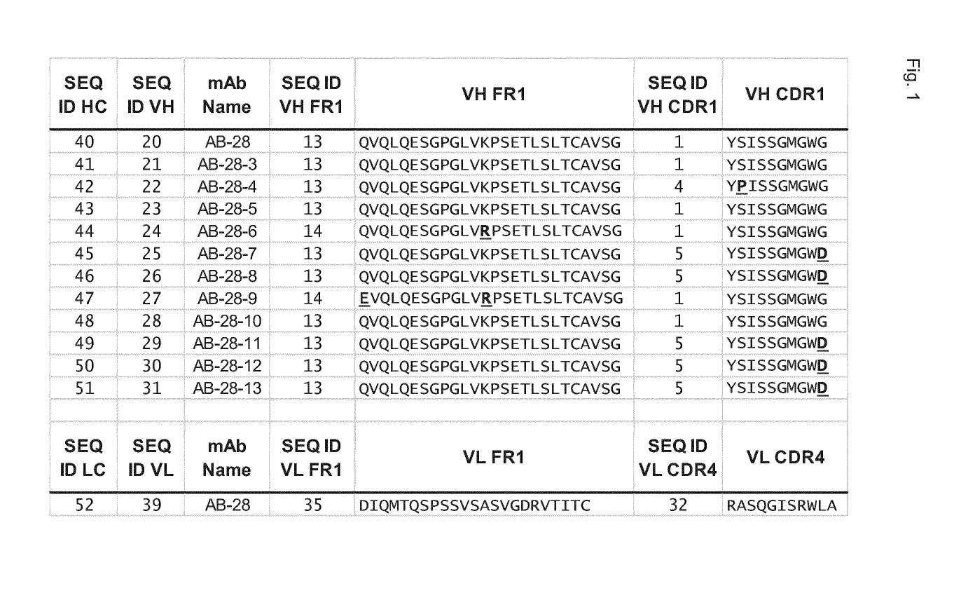

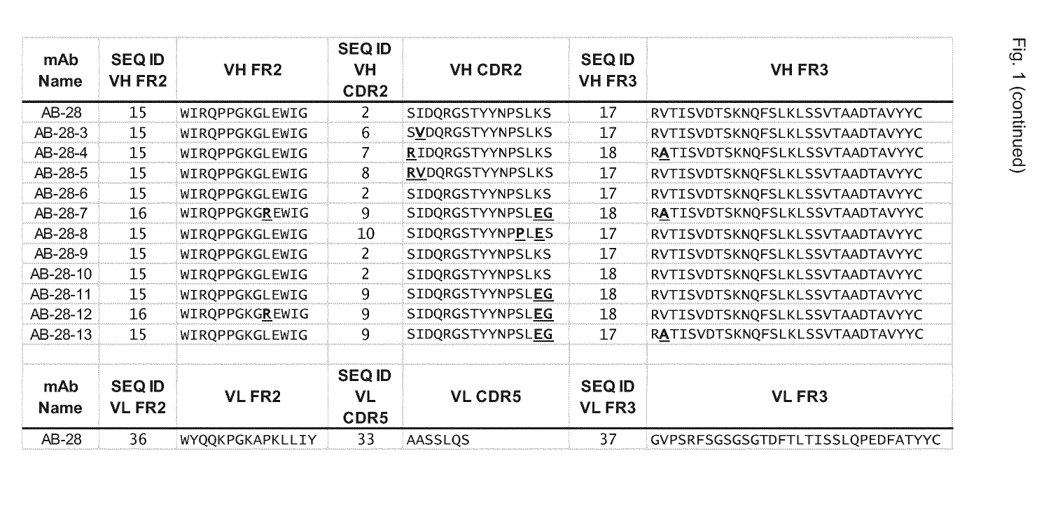

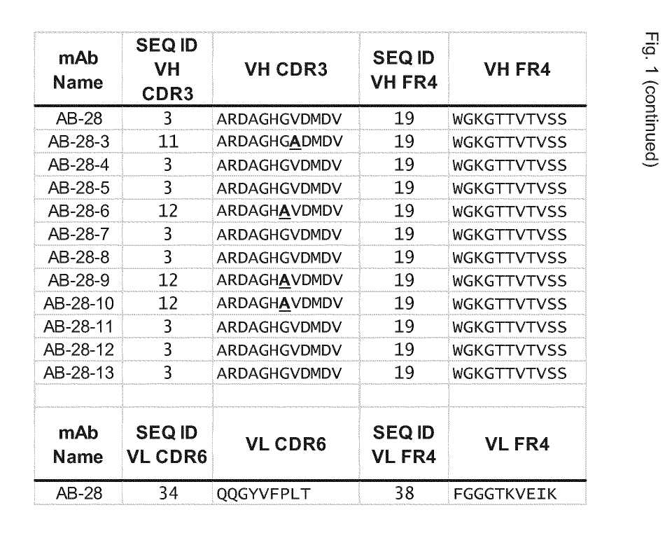

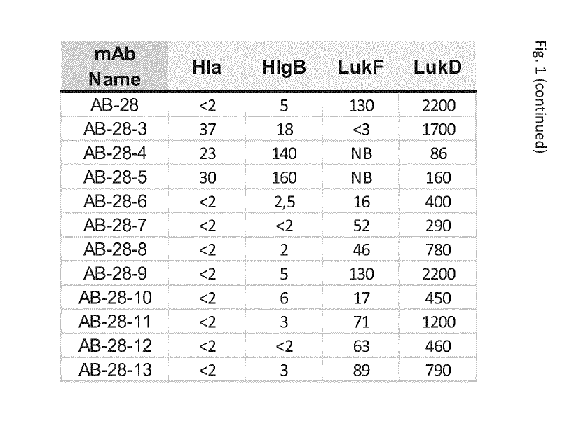

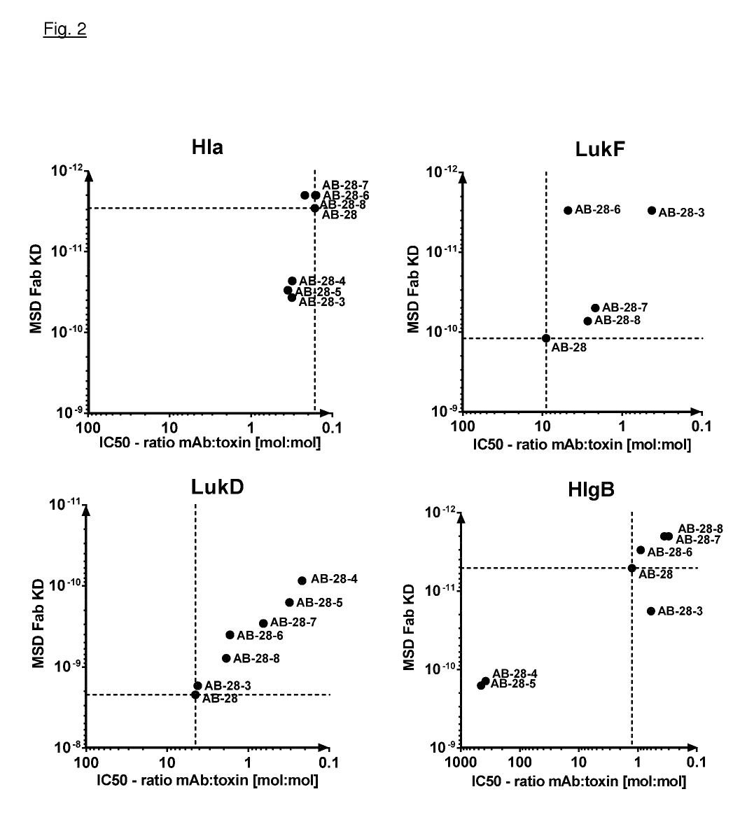

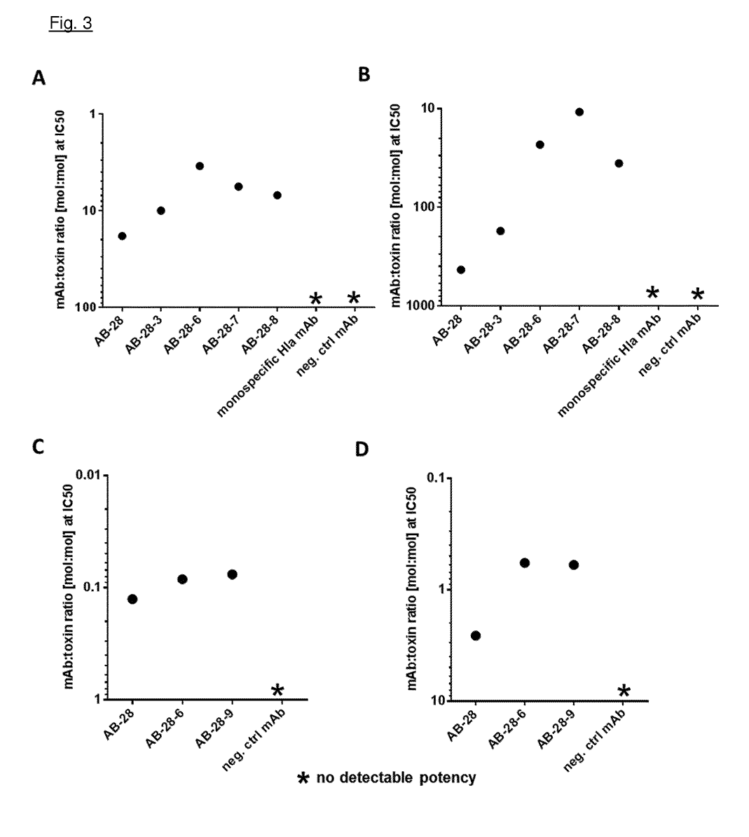

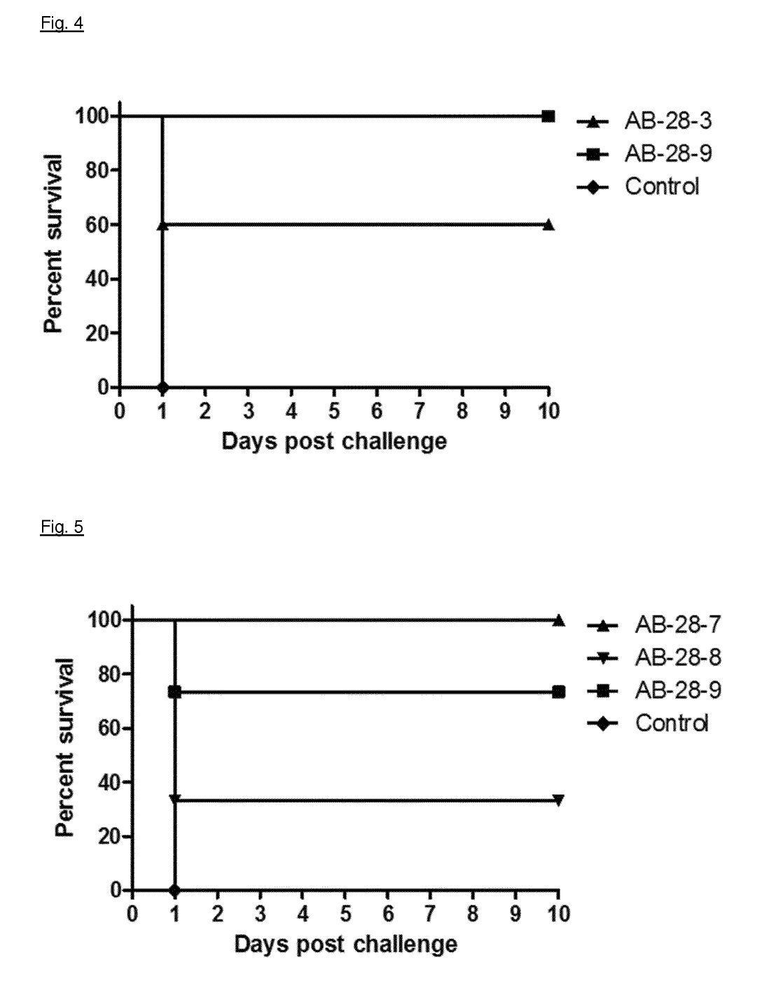

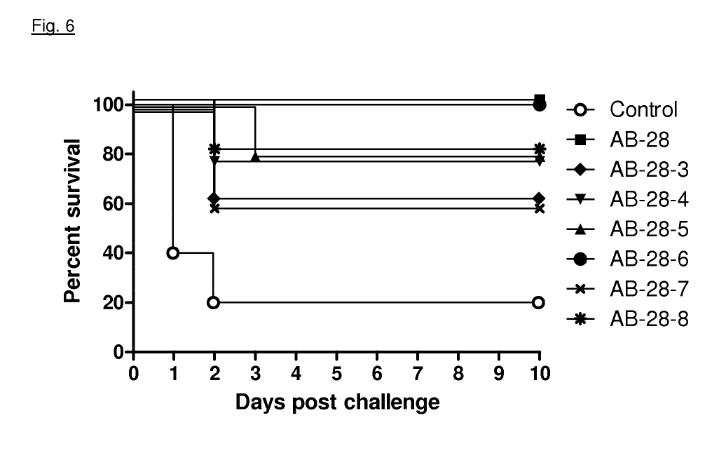

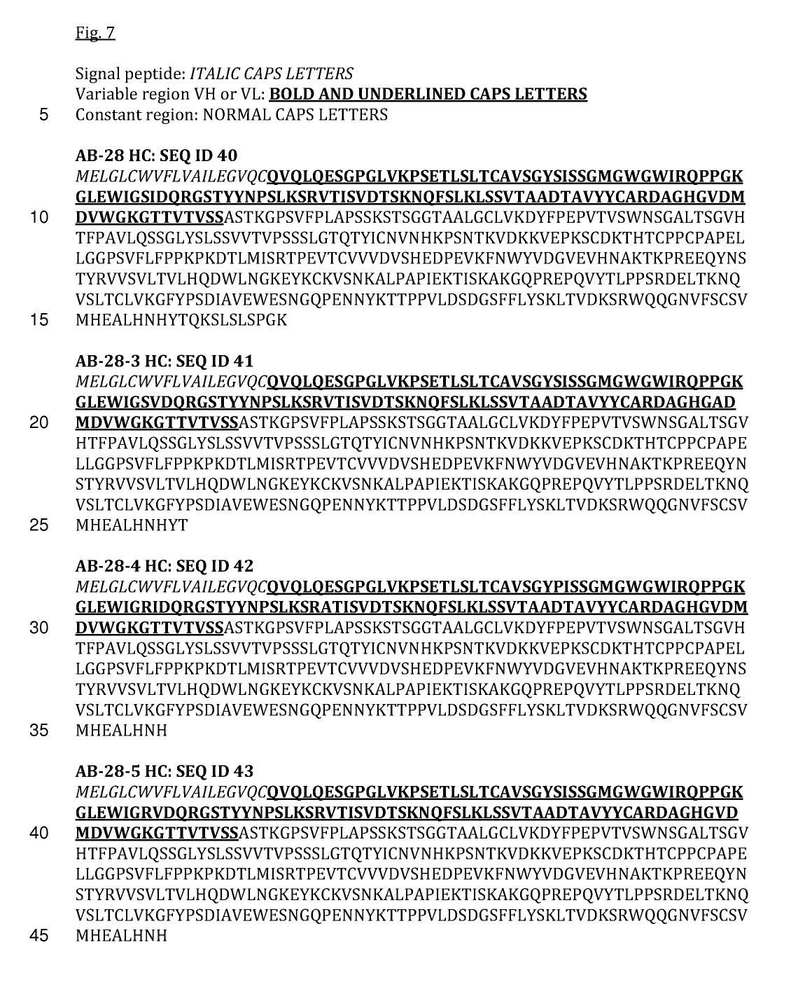

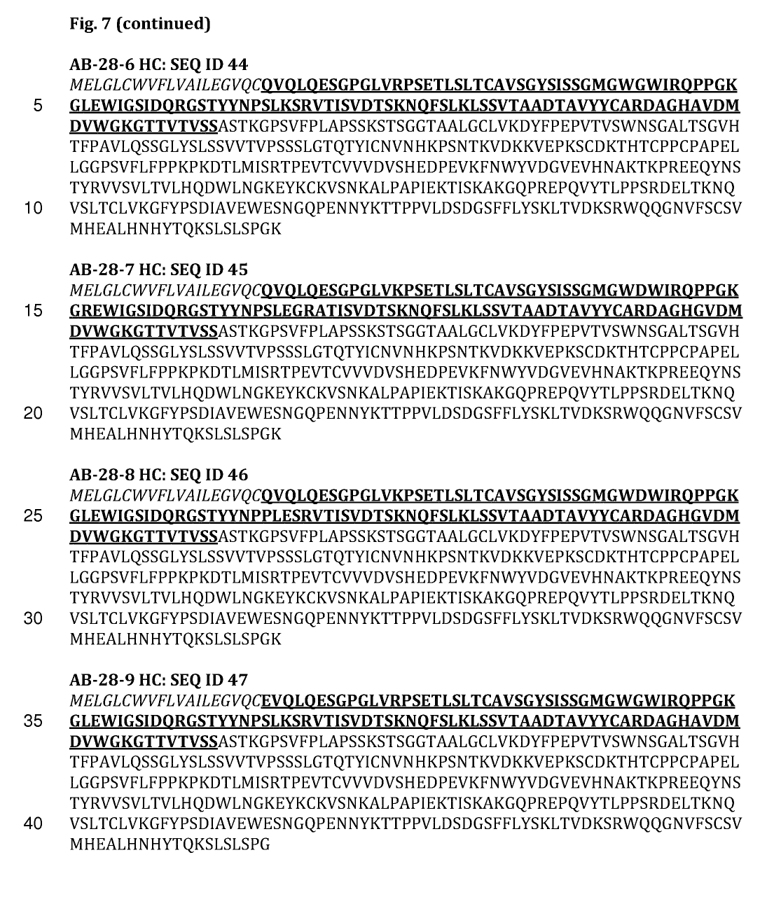

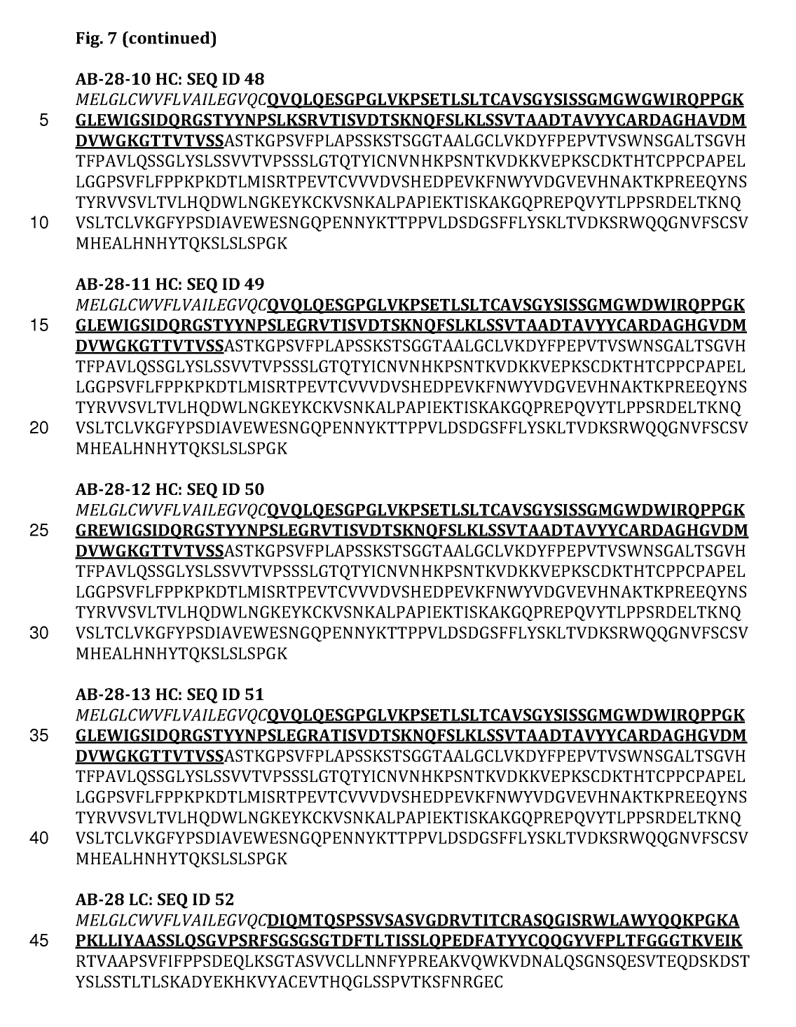

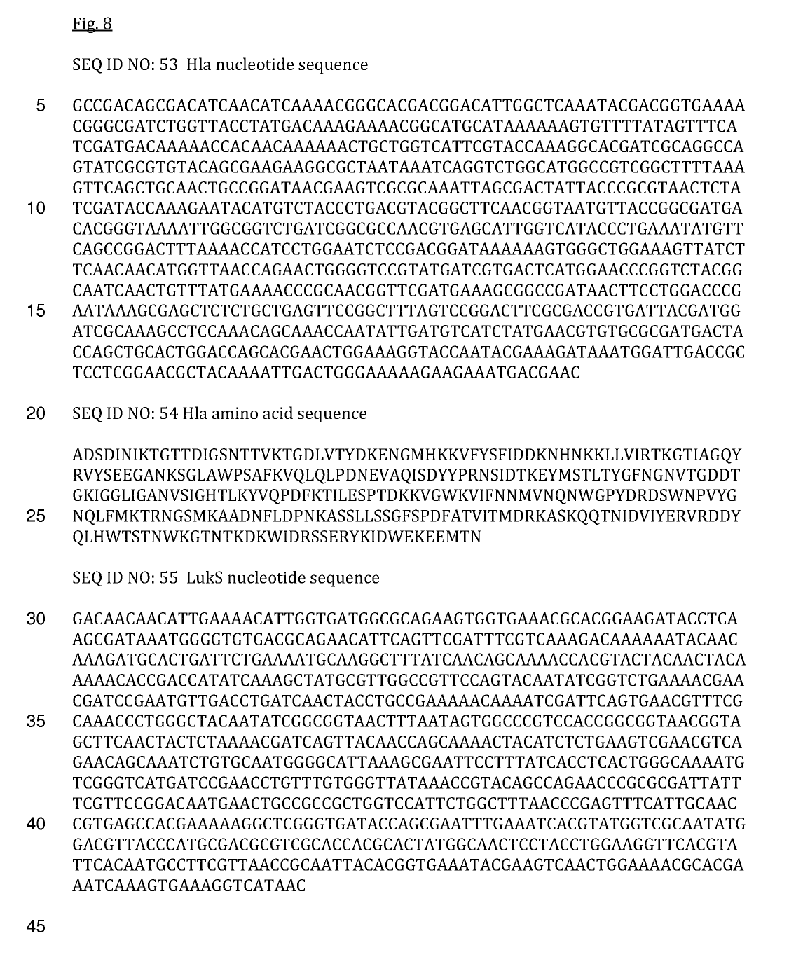

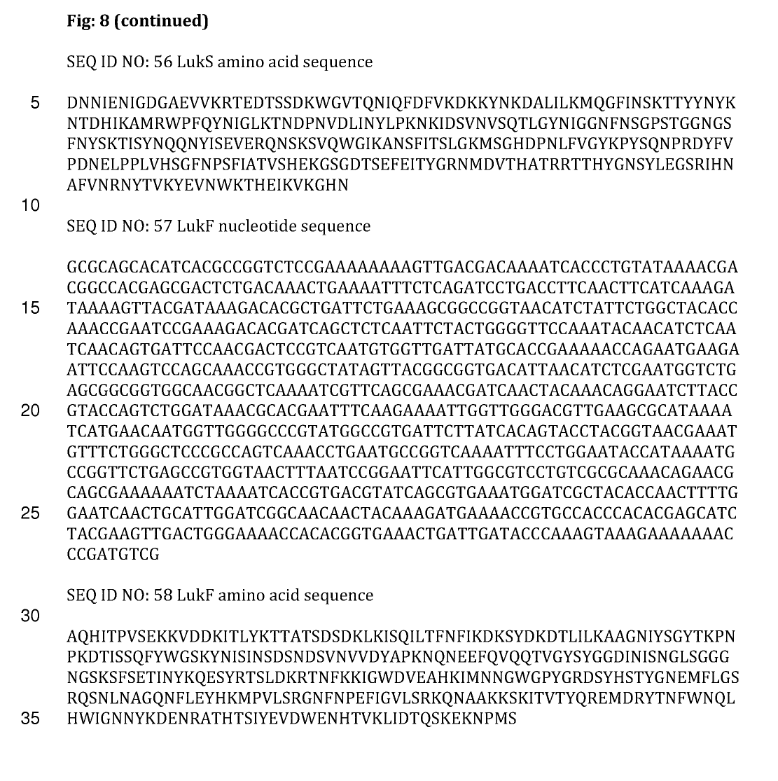

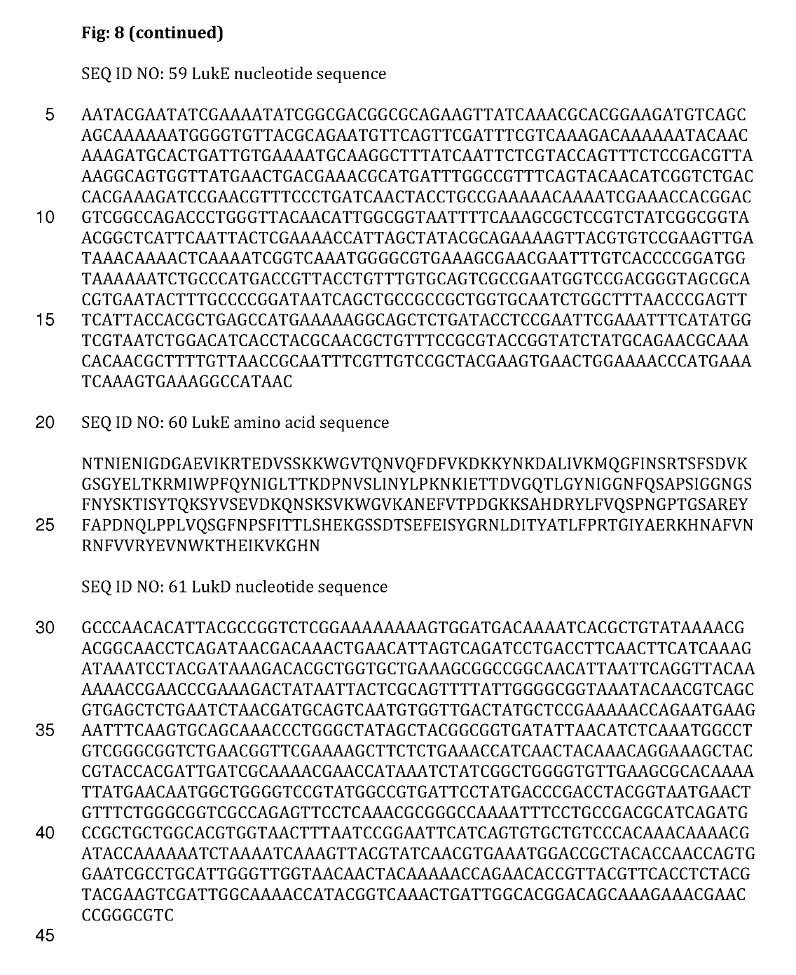

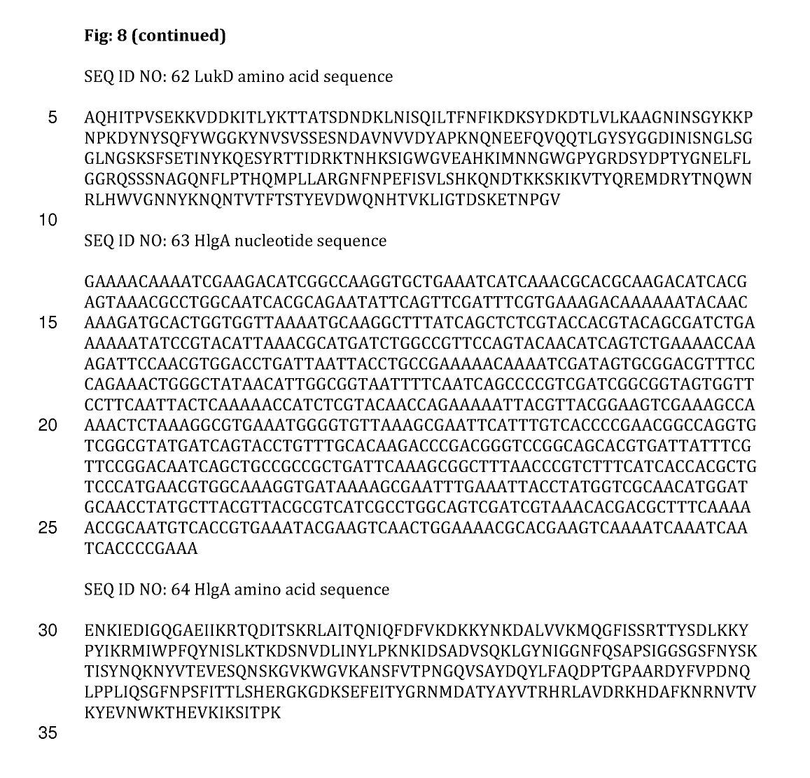

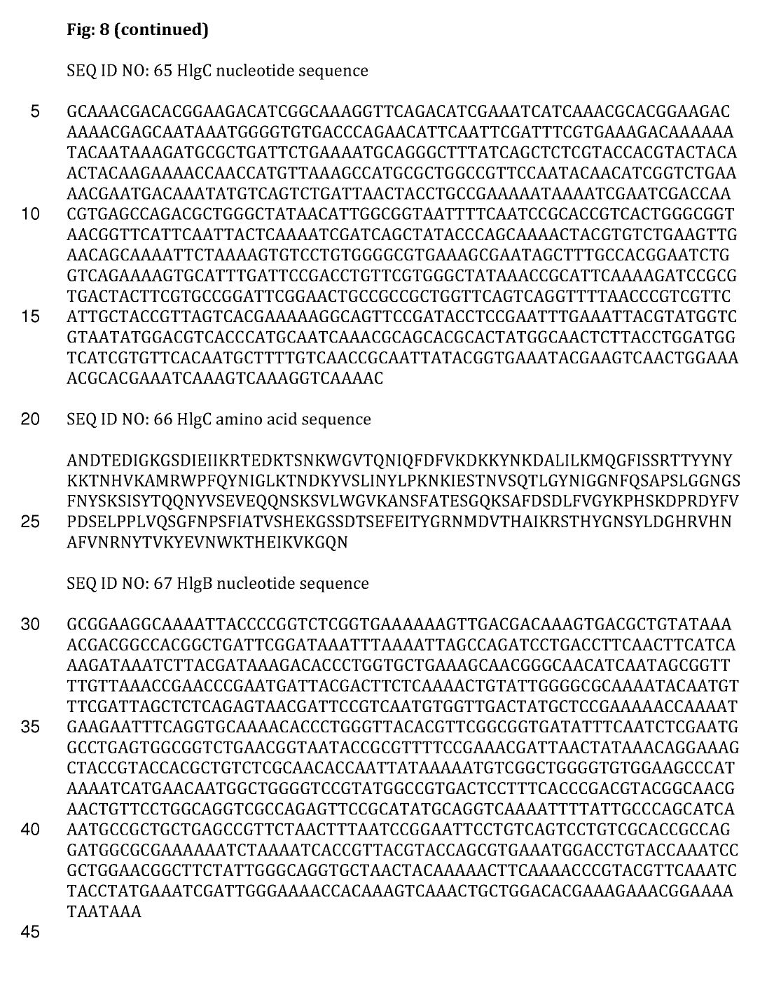

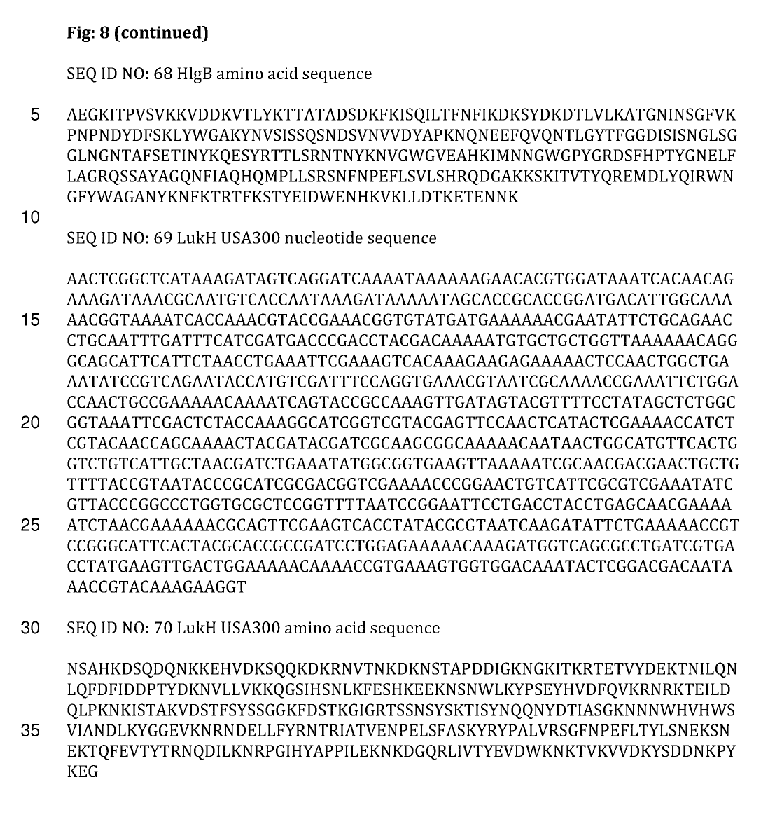

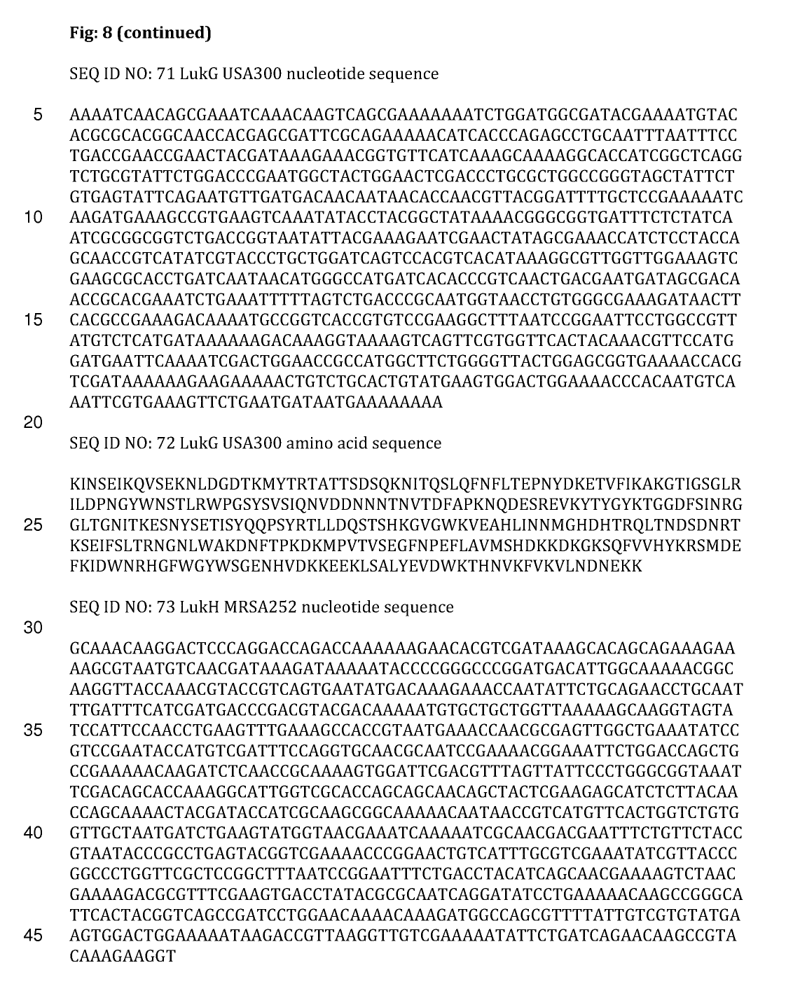

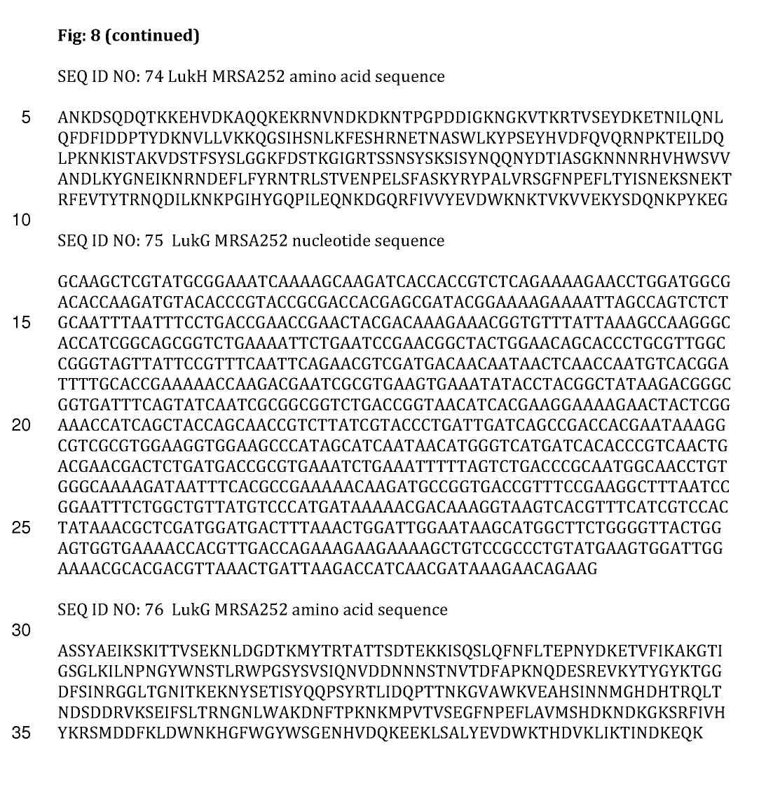

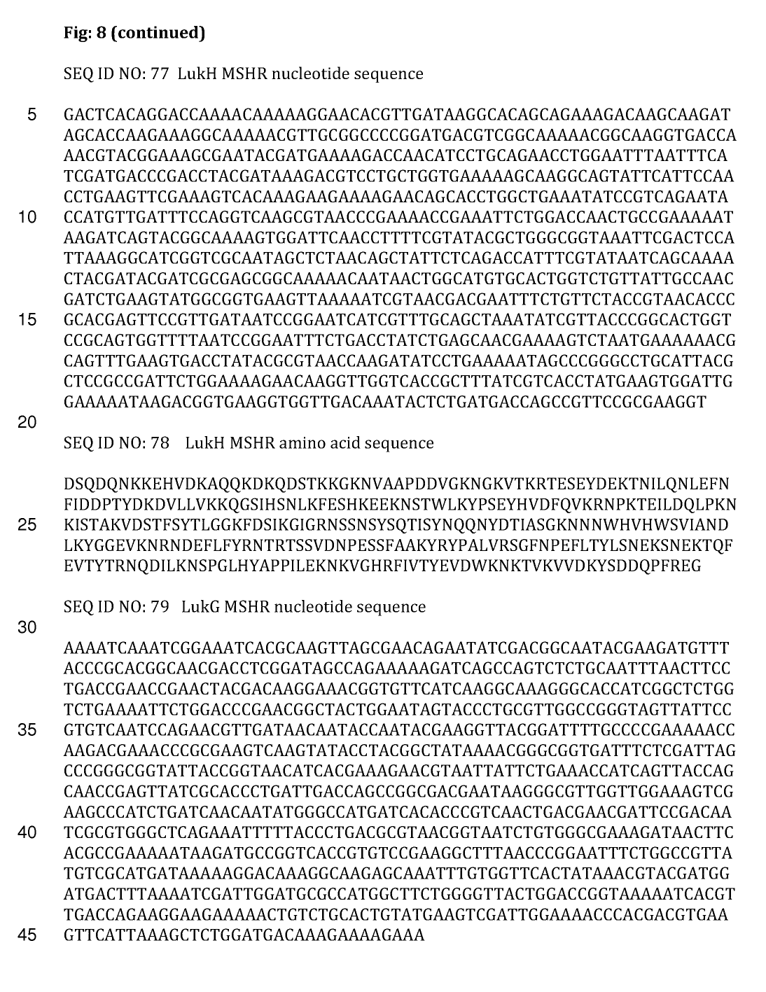

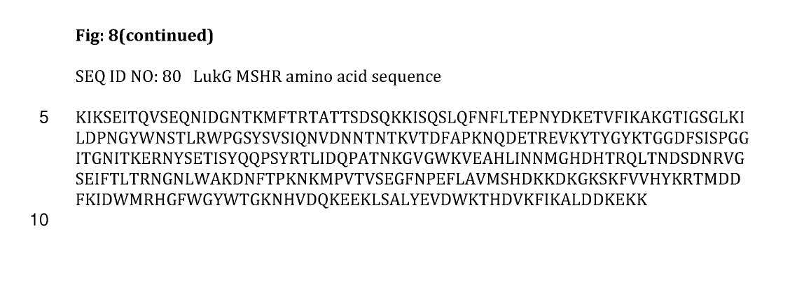

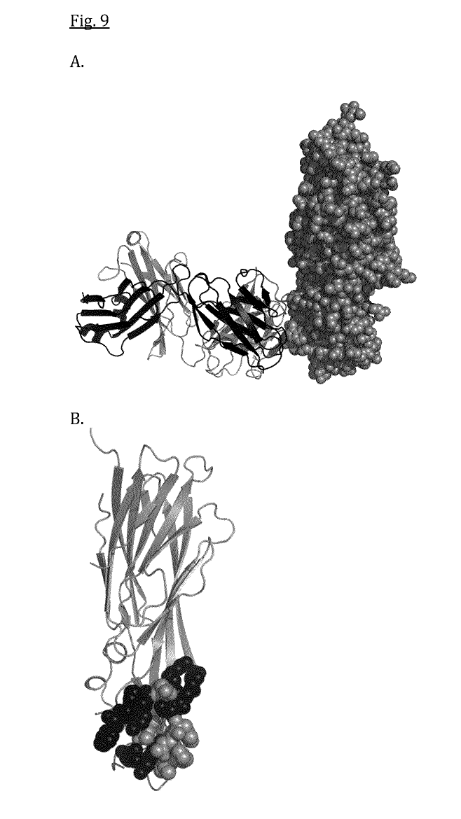

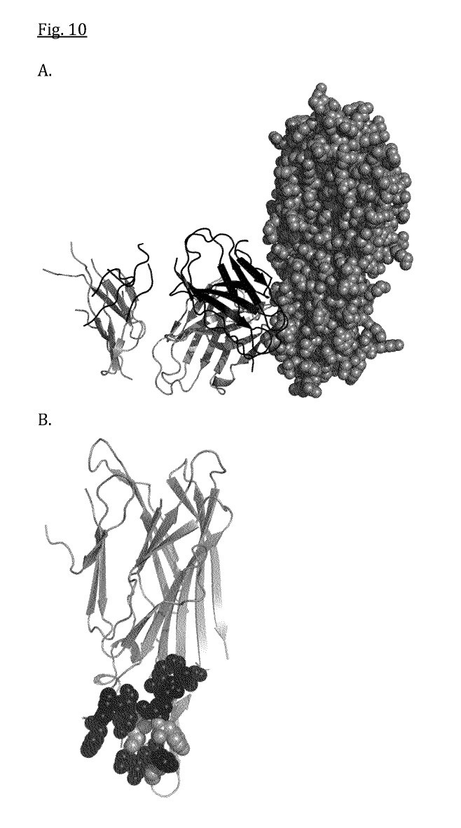

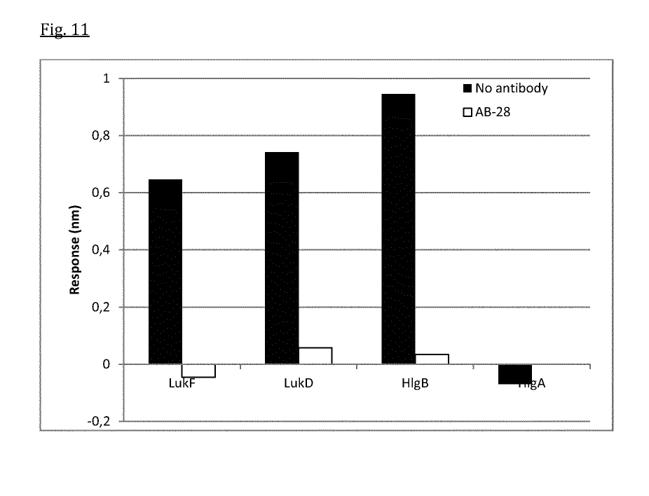

The nomenclature as used in FIG. 1 shall have the following meaning:

VH CDR1=CDR1

VH CDR2=CDR2

VH CDR3=CDR3

VL CDR4=CDR4=VL CDR1

VL CDR5=CDR5=VL CDR2

VL CDR6=CDR6=VL CDR3

FIG. 2. Toxin neutralizing potency of Hla—bi-component toxin cross reactive mAbs increased as affinities towards individual toxin components improved.

A: Hla [12.2 nM] hemoloysis inhibition assay on rabbit blood cells B: LukSF [2.94 nM] intoxication of human polymorphonuclear cells (PMNs), C: LukED [7.35 nM] intoxication of human PMNs, D: HlgCB [2.94 nM] intoxication of human PMNs. Y-axis: Fab KD affinities measured by MSD method using a Sector Immager 2400 instrument (Meso Scale Discovery). Typically 20 pM of biotinylated antigen was incubated with Fab at various concentrations, for 16 h at room temperature, and the unbound antigen captured on immobilized IgGSee also for example, Estep et al., “High throughput solution-based measurement of antibody-antigen affinity and epitope binning”, MAbs, Vol. 5(2), pp. 270-278 (2013). X-axis: IC50 values expressed as mAb to toxin molar ratio at half maximal inhibition of toxin mediated cell lysis. Affinity and potency levels of the parental AB-28 mAb are indicated by dotted lines.

FIG. 3: Hla—bi-component toxin cross reactive mAbs with high affinity to all targeted toxins efficiently inhibit cell lysis resulting from the combined effect of recombinant leukocidins.

A: Inhibition of lysis of human PMNs intoxicated with a mixture of HlgAB [2.94 nM], HlgCB [2.94 nM], LukSF [2.94 nM] and LukED [7.35 nM]. B: Inhibition of rabbit red blood cell hemolysis induced by treatment with a mixture of Hla [12.12 nM] and HlgAB [2.94 nM], HlgA-LukD [2.94 nM], LukED [2.94 nM] and LukSF [2.94 nM]. C: Hla [12.12 nM] hemolysis inhibition assay on rabbit red blood cells. D: Inhibition of lysis of human PMNs intoxicated with a mixture of Hla [3.03 nM], HlgCB [2.94 nM], LukSF [2.94 nM] and LukED [7.35 nM]. Potency of mAbs is expressed as mAb to toxin molar ratio at half maximal inhibition of toxin mediated cell lysis.* no detectable potency

FIG. 4: Protection by Hla—bi-component toxin cross reactive mAbs in murine HlgAB toxin challenge model

Mice were treated intraperitoneally with 200 μg (0.4 mg/ml) of indicated mAbs (10 mg/kg dose) 24 hr prior to intravenous challenge with a lethal dose of HlgA-HlgB toxin (both added at 0.2 μg/mouse dose). Survival of mice was followed for 10 days. Kaplan-Meier survival curves were found to be statistically significantly different by using the Logrank (Mantel-Cox) test. Control mice were treated with a human IgG1 generated against an irrelevant antigen.

FIG. 5: Protection by Hla—bi-component toxin cross reactive mAbs in murine HlgA-LukD toxin challenge model improved in parallel with higher LukD binding affinity

Mice were treated intraperitoneally with 100 μg (0.2 mg/ml) of indicated mAbs (5 mg/kg dose) 24 hr prior to intravenous challenge with a lethal dose of HlgA-LukD toxin (both added at 1 μg/mouse dose). Survival of mice was followed for 10 days. Kaplan-Meier survival curves were found to be statistically significantly different by using the Logrank (Mantel-Cox) test. Control mice were treated with a human IgG1 generated against an irrelevant antigen.

FIG. 6: Protection by Hla—bi-component toxin cross reactive mAbs in a murine pneumonia model.

Mice were treated intraperitoneally with 100 μg (0.2 mg/ml) of indicated mAbs (5 mg/kg dose) 24 hrs prior to intranasal challenge with a lethal dose of TCH1516 (6×10⁸ cfu in 40 μl corresponding to 1.5×10¹⁰ cfu/ml). Survival of mice was followed for 10 days. Kaplan-Meier survival curves were found to be statistically significantly different by using the Logrank (Mantel-Cox) test. Control mice were treated with vehicle only.

FIG. 7: Amino acid sequence information: HC of AB-28, AB-28-3, AB-28-4, AB-28-5, AB-28-6, AB-28-7, AB-28-8, AB-28-9, AB-28-10, AB-28-11, AB-28-12, AB-28-13 (SEQ ID 40-51), and LC of AB-28 (SEQ ID 52).

FIG. 8: S. aureus toxin sequences referred to herein.

SEQ ID 53 Hla nucleotide sequence of the USA300 TCH1516 strain (Genbank, accession number CP000730)

SEQ ID 54: Hla amino acid sequence of the USA300 TCH1516 strain

SEQ ID 55 LukS nucleotide sequence of the USA300 TCH1516 strain

SEQ ID 56: LukS amino acid sequence of the USA300 TCH1516 strain

SEQ ID 57 LukF nucleotide sequence of the USA300 TCH1516 strain

SEQ ID 58: LukF amino acid sequence of the USA300 TCH1516 strain

SEQ ID 59 LukE nucleotide sequence of the USA300 TCH1516 strain

SEQ ID 60: LukE amino acid sequence of the USA300 TCH1516 strain

SEQ ID 61 LukD nucleotide sequence of the USA300 TCH1516 strain

SEQ ID 62: LukD amino acid sequence of the USA300 TCH1516 strain

SEQ ID 63 HlgA nucleotide sequence of the USA300 TCH1516 strain

SEQ ID 64: HlgA amino acid sequence of the USA300 TCH1516 strain

SEQ ID 65 HlgC nucleotide sequence of the USA300 TCH1516 strain

SEQ ID 66: HlgC amino acid sequence of the USA300 TCH1516 strain

SEQ ID 67 HlgB nucleotide sequence of the USA300 TCH1516 strain

SEQ ID 68: HlgB amino acid sequence of the USA300 TCH1516 strain

SEQ ID 69: LukH nucleotide sequence of the USA300 TCH1516 strain

SEQ ID 70: LukH amino acid sequence of the USA300 TCH1516 strain

SEQ ID 71 LukG nucleotide sequence of the USA300 TCH1516 strain

SEQ ID 72: LukG amino acid sequence of the USA300 TCH1516 strain

SEQ ID 73 LukH nucleotide sequence of the MRSA252 strain (Genbank, accession number BX571856)

SEQ ID 74: LukH amino acid sequence of the MRSA252 strain

SEQ ID 75 LukG nucleotide sequence of the MRSA252 strain

SEQ ID 76: LukG amino acid sequence of the MRSA252 strain

SEQ ID 77 LukH nucleotide sequence of the MSHR1132 strain (Genbank, accession number FR821777)

SEQ ID 78: LukH amino acid sequence of the MSHR1132 strain

SEQ ID 79 LukG nucleotide sequence of the MSHR1132 strain

SEQ ID 80: LukG amino acid sequence of the MSHR1132 strain

FIG. 9:

A. Structure of Hla:AB-28 complex, with Hla represented as spheres and the Fab fragment of AB-28 as black cartoon for the light chain and grey carton for the heavy chain. B. Hla monomer with the AB-28 epitope (contact residues) shown as spheres (black for amino acids fully conserved between Hla, LukF, LukD and HlgB).

FIG. 10:

A. Structure of LukD:AB-28 complex, with LukD represented as spheres and the Fab fragment of AB-28 as black cartoon for the light chain and grey carton for the heavy chain. B. LukD monomer with the AB-28 epitope (contact residues) shown as spheres (black for amino acids fully conserved between Hla, LukF, LukD and HlgB)

FIG. 11:

Binding of phosphocholine (PC4-BSA) to biotinylated toxins in ForteBio in presence and in absence (No antibody) of AB-28.

### DETAILED DESCRIPTION

The term “antibody” as used herein shall refer to polypeptides or proteins that consist of or comprise antibody domains, which are understood as constant and/or variable domains of the heavy and/or light chains of immunoglobulins, with or without a linker sequence. Polypeptides are understood as antibody domains, if comprising a beta-barrel structure consisting of at least two beta-strands of an antibody domain structure connected by a loop sequence. Antibody domains may be of native structure or modified by mutagenesis or derivatization, e.g. to modify the antigen binding properties or any other property, such as stability or functional properties, such as binding to the Fc receptors FcRn and/or Fcgamma receptor.

The antibody as used herein has a specific binding site to bind one or more antigens or one or more epitopes of such antigens, specifically comprising a CDR binding site of a single variable antibody domain, such as VH, VL or VHH, or a binding site of pairs of variable antibody domains, such as a VLNH pair, an antibody comprising a VLNH domain pair and constant antibody domains, such as Fab, F(ab′), (Fab)₂, scFv, Fv, or a full length antibody.

The term “antibody” as used herein shall particularly refer to antibody formats comprising or consisting of single variable antibody domain, such as VH, VL or VHH, or combinations of variable and/or constant antibody domains with or without a linking sequence or hinge region, including pairs of variable antibody domains, such as a VLNH pair, an antibody comprising or consisting of a VLNH domain pair and constant antibody domains, such as heavy-chain antibodies, Fab, F(ab′), (Fab)₂, scFv, Fd, Fv, or a full-length antibody, e.g. of an IgG type (e.g., an IgG1, IgG2, IgG3, or IgG4 subtype), IgA1, IgA2, IgD, IgE, or IgM antibody. The term “full length antibody” can be used to refer to any antibody molecule comprising at least most of the Fc domain and other domains commonly found in a naturally occurring antibody monomer. This phrase is used herein to emphasize that a particular antibody molecule is not an antibody fragment.

The term “antibody” shall specifically include antibodies in the isolated form, which are substantially free of other antibodies directed against different target antigens or comprising a different structural arrangement of antibody domains. Still, an isolated antibody may be comprised in a combination preparation, containing a combination of the isolated antibody, e.g. with at least one other antibody, such as monoclonal antibodies or antibody fragments having different specificities.

The term “antibody” shall apply to antibodies of animal origin, including human species, such as mammalian, including human, murine, rabbit, goat, lama, cow and horse, or avian, such as hen, which term shall particularly include recombinant antibodies which are based on a sequence of animal origin, e.g. human sequences.

The term “antibody” further applies to chimeric antibodies with sequences of origin of different species, such as sequences of murine and human origin.

The term “chimeric” as used with respect to an antibody refers to those antibodies wherein one portion of each of the amino acid sequences of heavy and light chains is homologous to corresponding sequences in antibodies derived from a particular species or belonging to a particular class, while the remaining segment of the chain is homologous to corresponding sequences in another species or class. Typically the variable region of both light and heavy chains mimics the variable regions of antibodies derived from one species of mammals, while the constant portions are homologous to sequences of antibodies derived from another. For example, the variable region can be derived from presently known sources using readily available B-cells or hybridomas from non-human host organisms in combination with constant regions derived from, for example, human cell preparations.

The term “antibody” may further apply to humanized antibodies.

The term “humanized” as used with respect to an antibody refers to a molecule having an antigen binding site that is substantially derived from an immunoglobulin from a non-human species, wherein the remaining immunoglobulin structure of the molecule is based upon the structure and/or sequence of a human immunoglobulin. The antigen binding site may either comprise complete variable domains fused onto constant domains or only the complementarity determining regions (CDR) grafted onto appropriate framework regions in the variable domains. Antigen-binding sites may be wild-type or modified, e.g. by one or more amino acid substitutions, preferably modified to resemble human immunoglobulins more closely. Some forms of humanized antibodies preserve all CDR sequences (for example a humanized mouse antibody which contains all six CDRs from the mouse antibody). Other forms have one or more CDRs which are altered with respect to the original antibody.

The term “antibody” further applies to human antibodies.

The term “human” as used with respect to an antibody, is understood to include antibodies having variable and constant regions derived from human germline immunoglobulin sequences. The human antibody of the invention may include amino acid residues not encoded by human germline immunoglobulin sequences (e.g., mutations introduced by random or site-specific mutagenesis in vitro or by somatic mutation in vivo), for example in the CDRs. Human antibodies include antibodies isolated from human immunoglobulin libraries or from animals transgenic for one or more human immunoglobulin.

The term “antibody” specifically applies to antibodies of any class or subclass. Depending on the amino acid sequence of the constant domain of their heavy chains, antibodies can be assigned to the major classes of antibodies IgA, IgD, IgE, IgG, and IgM, and several of these may be further divided into subclasses (isotypes), e.g., IgG1, IgG2, IgG3, IgG4, IgA1, and IgA2.

The term further applies to monoclonal or polyclonal antibodies, specifically a recombinant antibody, which term includes all antibodies and antibody structures that are prepared, expressed, created or isolated by recombinant means, such as antibodies originating from animals, e.g. mammalians including human, that comprises genes or sequences from different origin, e.g. murine, chimeric, humanized antibodies, or hybridoma derived antibodies. Further examples refer to antibodies isolated from a host cell transformed to express the antibody, or antibodies isolated from a recombinant, combinatorial library of antibodies or antibody domains, or antibodies prepared, expressed, created or isolated by any other means that involve splicing of antibody gene sequences to other DNA sequences.

It is understood that the term “antibody” also refers to derivatives of an antibody, in particular functionally active derivatives. An antibody derivative is understood as any combination of one or more antibody domains or antibodies and/or a fusion protein, in which any domain of the antibody may be fused at any position of one or more other proteins, such as other antibodies, e.g. a binding structure comprising CDR loops, a receptor polypeptide, but also ligands, scaffold proteins, enzymes, toxins and the like. A derivative of the antibody may be obtained by association or binding to other substances by various chemical techniques such as covalent coupling, electrostatic interaction, di-sulphide bonding etc. The other substances bound to the antibody may be lipids, carbohydrates, nucleic acids, organic and inorganic molecules or any combination thereof (e.g. PEG, prodrugs or drugs). In a specific embodiment, the antibody is a derivative comprising an additional tag allowing specific interaction with a biologically acceptable compound. There is not a specific limitation with respect to the tag usable in the present invention, as far as it has no or tolerable negative impact on the binding of the antibody to its target. Examples of suitable tags include His-tag, Myc-tag, FLAG-tag, Strep-tag, Calmodulin-tag, GST-tag, MBP-tag, and S-tag. In another specific embodiment, the antibody is a derivative comprising a label. The term “label” as used herein refers to a detectable compound or composition which is conjugated directly or indirectly to the antibody so as to generate a “labeled” antibody. The label may be detectable by itself, e.g. radioisotope labels or fluorescent labels, or, in the case of an enzymatic label, may catalyze chemical alteration of a substrate compound or composition which is detectable.

The preferred derivatives as described herein are functionally active with regard to the antigen binding, and alike the antibodies that are not derivatized, preferably have a potency to neutralize S. aureus and/or which are protective antibodies.

Antibodies derived from a parent antibody or antibody sequence, such as a parent CDR or FR sequence, are herein particularly understood as mutants or variants obtained by e.g. in silico or recombinant engineering or else by chemical derivatization or synthesis.

Specifically, an antibody derived from an antibody of the invention may comprise at least one or more of the CDR regions or CDR variants thereof, e.g. at least 3 CDRs of the heavy chain variable region and/or at least 3 CDRs of the light chain variable region, with at least one point mutation in at least one of the CDR or FR regions, or in the constant region of the HC or LC, being functionally active, e.g. specifically binding the polyspecific binding site recognizing the toxins.

It is understood that the term “antibody” also refers to variants of an antibody, including antibodies with functionally active CDR variants of a parent CDR sequence, and functionally active variant antibodies of a parent antibody.

The term “variant” shall particularly refer to antibodies, such as mutant antibodies or fragments of antibodies, e.g. obtained by mutagenesis methods, in particular to delete, exchange, introduce inserts into a specific antibody amino acid sequence or region or chemically derivatize an amino acid sequence, e.g. in the constant domains to engineer the antibody stability, effector function or half-life, or in the variable domains to improve antigen-binding properties, e.g. by affinity maturation techniques available in the art. Any of the known mutagenesis methods may be employed, including point mutations at desired positions, e.g. obtained by randomisation techniques. In some cases positions are chosen randomly, e.g. with either any of the possible amino acids or a selection of preferred amino acids to randomise the antibody sequences. The term “mutagenesis” refers to any art recognized technique for altering a polynucleotide or polypeptide sequence. Preferred types of mutagenesis include error prone PCR mutagenesis, saturation mutagenesis, or other site directed mutagenesis.

The term “variant” shall specifically encompass functionally active variants.

The term “functionally active variant” of a CDR sequence as used herein, is understood as a “functionally active CDR variant”, and the “functionally active variant” of an antibody as used herein, is understood as “functionally active antibody variant”. The functionally active variant means a sequence resulting from modification of this sequence (a parent antibody or a parent sequence) by insertion, deletion or substitution of one or more amino acids, or chemical derivatization of one or more amino acid residues in the amino acid sequence, or nucleotides within the nucleotide sequence, or at either or both of the distal ends of the sequence, e.g. in a CDR sequence the N-terminal and/or C-terminal 1, 2, 3, or 4 amino acids, and/or the centric 1, 2, 3, or 4 amino acids (i.e. in the midst of the CDR sequence), and which modification does not affect, in particular impair, the activity of this sequence. In the case of a binding site having specificity to a selected target antigen, the functionally active variant of an antibody would still have the predetermined binding specificity, though this could be changed, e.g. to change the fine specificity to a specific epitope, the affinity, the avidity, the Kon or Koff rate, etc. For example, an affinity matured antibody is specifically understood as a functionally active variant antibody. Hence, the modified CDR sequence in an affinity matured antibody is understood as a functionally active CDR variant. Further modifications may be made through mutagenesis, e.g. to widen the cross-specificity to target more toxins or toxin components than the parent antibody, or to increase its reactivity with one or more of the toxins or toxin components.

Specifically, the functionally active variants of an antibody of the invention has the polyspecific binding site that binds to Hla and at least one of the bi-component toxins of S. aureus, as further described herein. A further indicator of functional activity shall be the competitive binding of any of the toxins to the cell membranes, in particular to phosphocholine.

The functional activity is preferably determined in an assay for determining the neutralization potency of antibodies against cytolytic toxins, e.g. determined in a standard assay by measuring increased viability or functionality of cells susceptible to the given toxin. For example, the functional activity is determined if the antibody exhibits in vitro neutralization potency in a cell-based assay with an IC50 of less than 100:1 mAb:toxin ratio (mol/mol), preferably less than 50:1, preferably less than 25:1, preferably less than 10:1, more preferably less than 1:1. Neutralization is typically expressed by percent of viable cells with and without antibodies. For highly potent antibodies, a preferred way of expressing neutralization potency is the antibody:toxin molar ratio, where lower values correspond to higher potency. Values below 10 define a high functional activity. Optionally, values are in the most stringent assay between 1 and 10.

Functionally active variants may be obtained, e.g. by changing the sequence of a parent antibody, e.g. an antibody comprising the binding site, i.e. the binding site formed by the CDR region, or formed by the VH with the sequence of SEQ ID 20, and the VL with the sequence of SEQ ID 39, or formed by the respective CDR region, which parent antibody or sequence is characterized by its cross-reactivity, but with modifications within an antibody region besides the binding site, or derived from such parent antibody, by a modification within the binding site, that does not impair the antigen binding, and preferably would have a biological activity similar to the parent antibody, including the ability to bind toxins of S. aureus and/or to neutralize S. aureus with a specific potency, e.g. with substantially the same biological activity, as determined by a specific binding assay or functional test to target S. aureus or S. aureus toxins.

Specifically, any of the following CDR sequences may be modified to include the following
a) in VH CDR1 at position 5, the amino acid residue is selected from the group consisting of S, A, D, E, F, G, H, I, K, L, M, N, Q, R, T V, W and Y, preferentially any of H, R and W;
b) in VH CDR1 at position 7, the amino acid residue is selected from the group consisting of M, H, K, Q, R and W, preferentially any of K, R or W;
c) in VH CDR2 at position 3, the amino acid residue is selected from the group consisting of D and R;
d) in VH CDR2 at position 7, the amino acid residue is selected from the group consisting of S, A, D, E, F, H, K, M, N, Q, R, T, W and Y, preferentially any of D, H, K, N or Q, and more preferentially is Q;
e) in VH CDR2 at position 9, the amino acid residue is selected from the group consisting of Y, F, K, L, Q and R, and preferentially is R;
f) in VH CDR3 at position 5, the amino acid residue is selected from the group consisting of G, A, D, F, H, I, M, N, R, S, T, V and Y, preferentially any of D, F, H, I, M, N, R, T, V or Y;
g) in VH CDR3 at position 6, the amino acid residue is selected from the group consisting of H, E, Q and S, preferentially any of E or Q;
h) in VH CDR3 at position 7, the amino acid residue is selected from the group consisting of G, A, D, E, H, I, M, N, Q, S, T, V and W, and preferentially is W; and/or
i) in VH CDR3 at position 8, the amino acid residue is selected from the group consisting of V, A, D, E, G, I, K, L, M, Q, R, S and T, preferentially any of M or R.

Specifically, any of the following CDR sequences may be modified to include the following
a) in VL CDR4 at position 7, the amino acid residue is selected from the group consisting of S, A, E, F, G, K, L, M, N, Q, R, W and Y, preferentially any of L, M, R or W, and more preferentially is R;
b) in VL CDR5 at position 1, the amino acid residue is selected from the group consisting of A and G;
c) in VL CDR5 at position 3, the amino acid residue is selected from the group consisting of S, A, D, G, H, I, K, L, N, Q, R, T, V and W;
d) in VL CDR5 at position 4, the amino acid residue is selected from the group consisting of S, D, E, H, I, K, M, N, Q, R, T and V, preferentially any of K, N, Q and R;
e) in VL CDR6 at position 3, the amino acid residue is selected from the group consisting of G, A, D, E, F, H, I, K, L, N, Q, R, S, T, V, W and Y;
f) in VL CDR6 at position 4, the amino acid residue is selected from the group consisting of Y, D, F, H, M, R and W;
g) in VL CDR6 at position 5, the amino acid residue is selected from the group consisting of V, A, D, E, F, G, H, I, K, L, M, N, Q, R, S, T, and W; and/or
h) in VL CDR6 at position 6, the amino acid residue is selected from the group consisting of F and W.

The term “substantially the same biological activity” as used herein refers to the activity as indicated by substantially the same activity being at least 20%, at least 50%, at least 75%, at least 90%, e.g. at least 100%, or at least 125%, or at least 150%, or at least 175%, or e.g. up to 200% of the activity as determined for the comparable or parent antibody.

The preferred variants or derivatives as described herein are functionally active with regard to the antigen binding, preferably which have a potency to specifically bind the individual toxins, and not significantly binding to other antigens that are not target toxins, e.g. with a Kd value difference of at least 2 logs, preferably at least 3 logs. The antigen binding by a functionally active variant is typically not impaired, corresponding to about substantially the same binding affinity as the parent antibody or sequence, or antibody comprising a sequence variant, e.g. with a Kd value difference of less than 2 logs, preferably less than 3 logs, however, with the possibility of even improved affinity, e.g. with a Kd value difference of at least 1 log, preferably at least 2 logs.

In a preferred embodiment the functionally active variant of a parent antibody

a) is a biologically active fragment of the antibody, the fragment comprising at least 50% of the sequence of the molecule, preferably at least 60%, at least 70%, at least 80%, at least 90%, or at least 95% and most preferably at least 97%, 98% or 99%;

b) is derived from the antibody by at least one amino acid substitution, addition and/or deletion, wherein the functionally active variant has a sequence identity to the molecule or part of it, such as an antibody of at least 50% sequence identity, preferably at least 60%, more preferably at least 70%, more preferably at least 80%, still more preferably at least 90%, even more preferably at least 95% and most preferably at least 97%, 98% or 99%; and/or

c) consists of the antibody or a functionally active variant thereof and additionally at least one amino acid or nucleotide heterologous to the polypeptide or the nucleotide sequence.

In one preferred embodiment of the invention, the functionally active variant of the antibody according to the invention is essentially identical to the variant described above, but differs from its polypeptide or the nucleotide sequence, respectively, in that it is derived from a homologous sequence of a different species. These are referred to as naturally occurring variants or analogs.

The term “functionally active variant” also includes naturally occurring allelic variants, as well as mutants or any other non-naturally occurring variants. As is known in the art, an allelic variant is an alternate form of a (poly) peptide that is characterized as having a substitution, deletion, or addition of one or more amino acids that does essentially not alter the biological function of the polypeptide.

Functionally active variants may be obtained by sequence alterations in the polypeptide or the nucleotide sequence, e.g. by one or more point mutations, wherein the sequence alterations retains or improves a function of the unaltered polypeptide or the nucleotide sequence, when used in combination of the invention. Such sequence alterations can include, but are not limited to, (conservative) substitutions, additions, deletions, mutations and insertions.

Specific functionally active variants are CDR variants. A CDR variant includes an amino acid sequence modified by at least one amino acid in the CDR region, wherein said modification can be a chemical or a partial alteration of the amino acid sequence, which modification permits the variant to retain the biological characteristics of the unmodified sequence. A partial alteration of the CDR amino acid sequence may be by deletion or substitution of one to several amino acids, e.g. 1, 2, 3, 4 or 5 amino acids, or by addition or insertion of one to several amino acids, e.g. 1, 2, 3, 4 or 5 amino acids, or by a chemical derivatization of one to several amino acids, e.g. 1, 2, 3, 4 or 5 amino acids, or combination thereof. The substitutions in amino acid residues may be conservative substitutions, for example, substituting one hydrophobic amino acid for an alternative hydrophobic amino acid.

Conservative substitutions are those that take place within a family of amino acids that are related in their side chains and chemical properties. Examples of such families are amino acids with basic side chains, with acidic side chains, with non-polar aliphatic side chains, with non-polar aromatic side chains, with uncharged polar side chains, with small side chains, with large side chains etc.

A point mutation is particularly understood as the engineering of a polynucleotide that results in the expression of an amino acid sequence that differs from the non-engineered amino acid sequence in the substitution or exchange, deletion or insertion of one or more single (non-consecutive) or doublets of amino acids for different amino acids.

Preferred point mutations refer to the exchange of amino acids of the same polarity and/or charge. In this regard, amino acids refer to twenty naturally occurring amino acids encoded by sixty-four triplet codons. These 20 amino acids can be split into those that have neutral charges, positive charges, and negative charges:

The “neutral” amino acids are shown below along with their respective three-letter and single-letter code and polarity:

Alanine: (Ala, A) nonpolar, neutral;

Asparagine: (Asn, N) polar, neutral;

Cysteine: (Cys, C) nonpolar, neutral;

Glutamine: (Gln, Q) polar, neutral;

Glycine: (Gly, G) nonpolar, neutral;

Isoleucine: (Ile, I) nonpolar, neutral;

Leucine: (Leu, L) nonpolar, neutral;

Methionine: (Met, M) nonpolar, neutral;

Phenylalanine: (Phe, F) nonpolar, neutral;

Proline: (Pro, P) nonpolar, neutral;

Serine: (Ser, S) polar, neutral;

Threonine: (Thr, T) polar, neutral;

Tryptophan: (Trp, W) nonpolar, neutral;

Tyrosine: (Tyr, Y) polar, neutral;

Valine: (Val, V) nonpolar, neutral; and

Histidine: (His, H) polar, positive (10%) neutral (90%).

The “positively” charged amino acids are:

Arginine: (Arg, R) polar, positive; and

Lysine: (Lys, K) polar, positive.

The “negatively” charged amino acids are:

Aspartic acid: (Asp, D) polar, negative; and

Glutamic acid: (Glu, E) polar, negative.

“Percent (%) amino acid sequence identity” with respect to the antibody sequences and homologs described herein is defined as the percentage of amino acid residues in a candidate sequence that are identical with the amino acid residues in the specific polypeptide sequence, after aligning the sequence and introducing gaps, if necessary, to achieve the maximum percent sequence identity, and not considering any conservative substitutions as part of the sequence identity. Those skilled in the art can determine appropriate parameters for measuring alignment, including any algorithms needed to achieve maximal alignment over the full length of the sequences being compared.

An antibody variant is specifically understood to include homologs, analogs, fragments, modifications or variants with a specific glycosylation pattern, e.g. produced by glycoengineering, which are functional and may serve as functional equivalents, e.g. binding to the specific targets and with functional properties. The preferred variants as described herein are functionally active with regard to the antigen binding, preferably which have a potency to neutralize S. aureus and/or which are protective antibodies.

An antibody of the present invention may or may not exhibit Fc effector function. Though the mode of action is mainly mediated by neutralizing antibodies without Fc effector functions, Fc can recruit complement and aid elimination of the target antigen, such as a toxin, from the circulation via formation of immune complexes.

Specific antibodies may be devoid of an active Fc moiety, thus, either composed of antibody domains that do not contain an Fc part of an antibody or that do not contain an Fcgamma receptor binding site, or comprising antibody domains lacking Fc effector function, e.g. by modifications to reduce Fc effector functions, in particular to abrogate or reduce ADCC and/or CDC activity. Alternative antibodies may be engineered to incorporate modifications to increase Fc effector functions, in particular to enhance ADCC and/or CDC activity.

Such modifications may be effected by mutagenesis, e.g. mutations in the Fcgamma receptor binding site or by derivatives or agents to interfere with ADCC and/or CDC activity of an antibody format, so to achieve reduction or increase of Fc effector function.

A significant reduction of Fc effector function is typically understood to refer to Fc effector function of less than 10% of the unmodified (wild-type) format, preferably less than 5%, as measured by ADCC and/or CDC activity.

A significant increase of Fc effector function is typically understood to refer to an increase in Fc effector function of at least 10% of the unmodified (wild-type) format, preferably at least 20%, 30%, 40% or 50%, as measured by ADCC and/or CDC activity.

The term “glycoengineered” variants with respect to antibody sequences shall refer to glycosylation variants having modified immunogenic or immunomodulatory (e.g. anti-inflammatory) properties, ADCC and/or CDC, as a result of the glycoengineering. All antibodies contain carbohydrate structures at conserved positions in the heavy chain constant regions, with each isotype possessing a distinct array of N-linked carbohydrate structures, which variably affect protein assembly, secretion or functional activity. IgG1 type antibodies are glycoproteins that have a conserved N linked glycosylation site at Asn297 in each CH2 domain. The two complex bi-antennary oligosaccharides attached to Asn297 are buried between the CH2 domains, forming extensive contacts with the polypeptide backbone, and their presence is essential for the antibody to mediate effector functions such as antibody dependent cellular cytotoxicity (ADCC). Removal of N-Glycan at N297, e.g. through mutating N297, e.g. to A, or T299 typically results in aglycosylated antibody formats with reduced ADCC. Specifically, the antibody of the invention may be glycosylated or glycoengineered, or aglycosylated antibodies.

Major differences in antibody glycosylation occur between cell lines, and even minor differences are seen for a given cell line grown under different culture conditions. Expression in bacterial cells typically provides for an aglycosylated antibody. CHO cells with tetracycline-regulated expression of β(1,4)-N-acetylglucosaminyltransferase III (GnTIII), a glycosyltransferase catalyzing formation of bisecting GlcNAc, was reported to have improved ADCC activity (Umana et al., 1999, Nature Biotech. 17:176-180). In addition to the choice of host cells, factors that affect glycosylation during recombinant production of antibodies include growth mode, media formulation, culture density, oxygenation, pH, purification schemes and the like.

The term “antigen-binding site” or “binding site” refers to the part of an antibody that participates in antigen binding. The antigen binding site is formed by amino acid residues of the N-terminal variable (“V”) regions of the heavy (“H”) and/or light (“L”) chains, or the variable domains thereof. Three highly divergent stretches within the V regions of the heavy and light chains, referred to as “hypervariable regions”, are inter-posed between more conserved flanking stretches known as framework regions, The antigen-binding site provides for a surface that is complementary to the three-dimensional surface of a bound epitope or antigen, and the hypervariable regions are referred to as “complementarity-determining regions”, or “CDRs.” The binding site incorporated in the CDRs is herein also called “CDR binding site”.

Specifically, the CDR sequences as referred to herein are understood as those amino acid sequences of an antibody as determined according to Kabat nomenclature (see Kabat et al., Sequences of Proteins of Immunological Interest, 5^{th} Ed. Public Health Service, U.S. Department of Health and Human Services. (1991)).

The term “antigen” as used herein interchangeably with the terms “target” or “target antigen” shall refer to a whole target molecule or a fragment of such molecule recognized by an antibody binding site. Specifically, substructures of an antigen, e.g. a polypeptide or carbohydrate structure, generally referred to as “epitopes”, e.g. B-cell epitopes or T-cell epitope, which are immunologically relevant, may be recognized by such binding site.

The term “epitope” as used herein shall in particular refer to a molecular structure which may completely make up a specific binding partner or be part of a specific binding partner to a binding site of an antibody. An epitope may either be composed of a carbohydrate, a peptidic structure, a fatty acid, an organic, biochemical or inorganic substance or derivatives thereof and any combinations thereof. If an epitope is comprised in a peptidic structure, such as a peptide, a polypeptide or a protein, it will usually include at least 3 amino acids, preferably 5 to 40 amino acids, and more preferably between about 10-20 amino acids. Epitopes can be either linear or conformational epitopes. A linear epitope is comprised of a single segment of a primary sequence of a polypeptide or carbohydrate chain. Linear epitopes can be contiguous or overlapping.

Conformational epitopes are comprised of amino acids or carbohydrates brought together by folding the polypeptide to form a tertiary structure and the amino acids are not necessarily adjacent to one another in the linear sequence. Specifically and with regard to polypeptide antigens a conformational or discontinuous epitope is characterized by the presence of two or more discrete amino acid residues, separated in the primary sequence, but assembling to a consistent structure on the surface of the molecule when the polypeptide folds into the native protein/antigen. Specifically, the conformational epitope is an epitope which is comprised of a series of amino acid residues which are non-linear in alignment that is that the residues are spaced or grouped in a non-continuous manner along the length of a polypeptide sequence. Such conformational epitope is characterized by a three-dimensional structure with specific structure coordinates as determined by contacting amino acid residues and/or crystallographic analysis, e.g. analysis of a crystal formed by the immune complex of the epitope cound by a specific antibody or Fab fragment.

In particular, the binding residues which contribute to an epitope are herein understood as the contacting amino acid residues.

The term “structure coordinates” refers to Cartesian atomic coordinates derived from mathematical equations related to the patterns obtained on diffraction of a monochromatic beam of X-rays by the atoms, i.e. scattering centres, of the antigen or the immune complex in a crystal form. The diffraction data are used to calculate an electron density map of the repeating unit of the crystal. The electron density maps are then used to establish the individual atoms of the epitope, e.g. as bound by the specific antibody or Fab. It is understood that a set of structure coordinates for an antigen is a relative set of points that define a shape in three dimensions. Slight variations in the individual coordinates will have little effect on overall shape. For the purpose of the invention, any three-dimensional structure that has a root mean square deviation of conserved residue backbone atoms between 0.00 A and 2.00 A when superimposed on the relevant backbone atoms described by the structure coordinates of the antigen are considered identical or substantially identical. For the purpose of the invention, structure coordinates are considered identical or substantially identical even if slight variations are present in the individual coordinates if these do not affect the overall shape defined by the structure coordinates.

Herein the term “epitope” shall particularly refer to the single epitope recognized by an antibody, or the mixture of epitopes comprising epitope variants, each recognized by a cross-reactive antibody.

The cross-reactive antibody as described herein is specifically recognizing the rim domain of the toxins, in particular the soluble toxin monomers or toxin components. The rim domain is understood as the domain of the toxin that is juxtaposed to the outer leaflet of the host plasma membrane, which rim domain is involved in cell membrane binding. Thus, the rim region serves as a membrane anchor. The epitope targeted by the antibody of the invention, which is located in the rim region or the rim domain, thus, has the potential of being immunorelevant, i.e. relevant for protection by active or passive immunotherapy.

Based on the identified epitope of the invention, it is feasible to additionally provide for a respective paratope, e.g. the paratope of an antibody of the invention, such as the part of a full length antibody which recognizes the epitope, the antigen-binding site of an antibody. It is typically a small region of 15-22 amino acids of the antibody's Fv region. Such paratope may be incoroporated in a suitable scaffold to obtain a scaffold-type molecule, e.g. a fusion protein or artificial scaffolds, to obtain a specific binder or binding molecule with the desired cross-reactive binding characteristics.

The term “binding molecule” as used herein is understood as an epitope-binding molecule or an antigen-binding molecule that specifically recognizes the target, and in particular exhibiting cross-specificity to the target toxins. Specific examples of binding molecules are selected from the group consisting of a protein, a peptide, a peptidomimetic, a nucleic acid, a carbohydrate, a lipid, an oligopeptide, an aptamer and a small molecule compound, preferably an antibody, an antibody fragment thereof comprising at least one antibody domain incorporating the binding site, or a fusion protein comprising at least one antibody domain incorporating the binding site.

A binding molecule may e.g. be selected from suitable libraries of binders, e.g. antibody libraries, or libraries of other compounds or scaffolds, e.g. DARPins, HEAT repeat proteins, ARM repeat proteins, tetratricopeptide repeat proteins, and other scaffolds based on naturally occurring repeat proteins, by suitable screening methods to obtain a candidate compound, which is then further characterized for its binding characteristics.

The term “expression” is understood in the following way. Nucleic acid molecules containing a desired coding sequence of an expression product such as e.g. an antibody as described herein, and control sequences such as e.g. a promoter in operable linkage, may be used for expression purposes. Hosts transformed or transfected with these sequences are capable of producing the encoded proteins. In order to effect transformation, the expression system may be included in a vector; however, the relevant DNA may also be integrated into the host chromosome. Specifically the term refers to a host cell and compatible vector under suitable conditions, e.g. for the expression of a protein coded for by foreign DNA carried by the vector and introduced to the host cell.

Coding DNA is a DNA sequence that encodes a particular amino acid sequence for a particular polypeptide or protein such as e.g. an antibody. Promoter DNA is a DNA sequence which initiates, regulates, or otherwise mediates or controls the expression of the coding DNA. Promoter DNA and coding DNA may be from the same gene or from different genes, and may be from the same or different organisms. Recombinant cloning vectors will often include one or more replication systems for cloning or expression, one or more markers for selection in the host, e.g. antibiotic resistance, and one or more expression cassettes.

“Vectors” used herein are defined as DNA sequences that are required for the transcription of cloned recombinant nucleotide sequences, i.e. of recombinant genes and the translation of their mRNA in a suitable host organism.

An “expression cassette” refers to a DNA coding sequence or segment of DNA that code for an expression product that can be inserted into a vector at defined restriction sites. The cassette restriction sites are designed to ensure insertion of the cassette in the proper reading frame. Generally, foreign DNA is inserted at one or more restriction sites of the vector DNA, and then is carried by the vector into a host cell along with the transmissible vector DNA. A segment or sequence of DNA having inserted or added DNA, such as an expression vector, can also be called a “DNA construct”.

Expression vectors comprise the expression cassette and additionally usually comprise an origin for autonomous replication in the host cells or a genome integration site, one or more selectable markers (e.g. an amino acid synthesis gene or a gene conferring resistance to antibiotics such as zeocin, kanamycin, G418 or hygromycin), a number of restriction enzyme cleavage sites, a suitable promoter sequence and a transcription terminator, which components are operably linked together. The term “vector” as used herein includes autonomously replicating nucleotide sequences as well as genome integrating nucleotide sequences. A common type of vector is a “plasmid”, which generally is a self-contained molecule of double-stranded DNA that can readily accept additional (foreign) DNA and which can readily be introduced into a suitable host cell. A plasmid vector often contains coding DNA and promoter DNA and has one or more restriction sites suitable for inserting foreign DNA. Specifically, the term “vector” or “plasmid” refers to a vehicle by which a DNA or RNA sequence (e.g. a foreign gene) can be introduced into a host cell, so as to transform the host and promote expression (e.g. transcription and translation) of the introduced sequence.

The term “host cell” as used herein shall refer to primary subject cells transformed to produce a particular recombinant protein, such as an antibody as described herein, and any progeny thereof. It should be understood that not all progeny are exactly identical to the parental cell (due to deliberate or inadvertent mutations or differences in environment), however, such altered progeny are included in these terms, so long as the progeny retain the same functionality as that of the originally transformed cell. The term “host cell line” refers to a cell line of host cells as used for expressing a recombinant gene to produce recombinant polypeptides such as recombinant antibodies. The term “cell line” as used herein refers to an established clone of a particular cell type that has acquired the ability to proliferate over a prolonged period of time. Such host cell or host cell line may be maintained in cell culture and/or cultivated to produce a recombinant polypeptide.

An “immune response” to a composition, e.g. an immunogenic composition, herein also termed “immunogen” comprising an antigen or epitope, or a vaccine as described herein is the development in the host or subject of a cellular- and/or antibody-mediated immune response to the composition or vaccine of interest. Usually, such a response consists of the subject producing antibodies, B cells, helper T cells, suppressor T cells, and/or cytotoxic T cells directed specifically to an antigen or antigens included in the composition or vaccine of interest.

A “protective immune response” is understood as an immune response that provides a significantly better outcome of an induced or natural infection or toxin challenge in comparison to that of the non-immune population. Protective immune response against toxins is mainly mediated by neutralizing antibodies having high affinity, e.g. with a Kd of less than 10⁻⁸M. The benefit of neutralization of toxins is the protection of targets cells and prevention of inflammation. Fc mediated immune complex formation can contribute as well by removing the toxin from the circulation (via the RES cells).

An immunogen or immunogenic composition usually comprises the antigen or epitope and a carrier, which may specifically comprise an adjuvant. The term “adjuvant” refers to a compound that when administered in conjunction with an antigen augments and/or redirects the immune response to the antigen, but when administered alone does not generate an immune response to the antigen. Adjuvants can augment an immune response by several mechanisms including lymphocyte recruitment, stimulation of B and/or T cells, and stimulation of macrophages. Exemplary carriers are liposomes or cationic peptides; exemplary adjuvants are aluminium phosphate or aluminium hydroxide, MF59 or CpG oligonucleotide.

The term “isolated” or “isolation” as used herein with respect to a nucleic acid, an antibody or other compound shall refer to such compound that has been sufficiently separated from the environment with which it would naturally be associated, so as to exist in “substantially pure” form. “Isolated” does not necessarily mean the exclusion of artificial or synthetic mixtures with other compounds or materials, or the presence of impurities that do not interfere with the fundamental activity, and that may be present, for example, due to incomplete purification. In particular, isolated nucleic acid molecules of the present invention are also meant to include those chemically synthesized.

With reference to nucleic acids of the invention, the term “isolated nucleic acid” is sometimes used. This term, when applied to DNA, refers to a DNA molecule that is separated from sequences with which it is immediately contiguous in the naturally occurring genome of the organism in which it originated. For example, an “isolated nucleic acid” may comprise a DNA molecule inserted into a vector, such as a plasmid or virus vector, or integrated into the genomic DNA of a prokaryotic or eukaryotic cell or host organism. When applied to RNA, the term “isolated nucleic acid” refers primarily to an RNA molecule encoded by an isolated DNA molecule as defined above. Alternatively, the term may refer to an RNA molecule that has been sufficiently separated from other nucleic acids with which it would be associated in its natural state (i.e., in cells or tissues). An “isolated nucleic acid” (either DNA or RNA) may further represent a molecule produced directly by biological or synthetic means and separated from other components present during its production.

With reference to polypeptides or proteins, such as isolated antibodies or epitopes of the invention, the term “isolated” shall specifically refer to compounds that are free or substantially free of material with which they are naturally associated such as other compounds with which they are found in their natural environment, or the environment in which they are prepared (e g. cell culture) when such preparation is by recombinant DNA technology practiced in vitro or in vivo. Isolated compounds can be formulated with diluents or adjuvants and still for practical purposes be isolated—for example, the polypeptides or polynucleotides can be mixed with pharmaceutically acceptable carriers or excipients when used in diagnosis or therapy.

The term “neutralizing” or “neutralization” is used herein in the broadest sense and refers to any molecule that inhibits a pathogen, such as S. aureus from infecting a subject, or to inhibit the pathogen from promoting infections by producing potent protein toxins, or to inhibit the toxins from damaging a target cell in a subject, irrespective of the mechanism by which neutralization is achieved. Neutralization can be achieved, e.g., by an antibody that inhibits the binding and/or interaction of the S. aureus toxin(s) with its cognate receptor on target cells. In certain embodiments, the antibodies described herein can neutralize the toxin activity wherein the in vivo or in vitro effects of the interaction between the toxin and the target cell, such as red blood cells are reduced or eliminated. Neutralization can further occur by inhibition of forming active toxin, for example in the case of the S. aureus bi-component cytolysins, by inhibition of binding of the S- and F-components or formation of the oligomeric pores in cytomembranes.

The neutralization potency of antibodies against cytolytic toxins is typically determined in a standard assay by measuring increased viability or functionality of cells susceptible to the given toxin. Neutralization can be expressed by percent of viable cells with and without antibodies. For highly potent antibodies, a preferred way of expressing neutralization potency is the antibody:toxin molar ratio, where lower values correspond to higher potency. Values below 10 define high, while values below 1 define very high potency.

The term “cross-neutralizing” as used herein shall refer to neutralizing a number of toxins, e.g. toxins incorporating a cross-reactive epitope recognized by the cross-reactive or polyspecific antibody.

The term “Staphylococcus aureus” or “S. aureus” or “pathogenic S. aureus” is understood in the following way. Staphylococcus aureus bacteria are normally found on the skin or in the nose of people and animals. The bacteria are generally harmless, unless they enter the body through a cut or other wound. Typically, infections are minor skin problems in healthy people. Historically, infections were treated by broad-spectrum antibiotics, such as methicillin. Now, though, certain strains have emerged that are resistant to methicillin and other beta-lactam antibiotics, such as penicillin and cephalosporins. They are referred to as methicillin-resistant Staphylococcus aureus (also known as multi-drug resistant Staphylococcus aureus, or “MRSA”).

Staphylococcus aureus, an important human pathogen, expresses a multitude of secreted toxins (exotoxins). These can attack various host cell types, including erythrocytes, neutrophil granulocytes and other immune cells, as well as epithelial cells of the lung or skin. A prominent member of S. aureus toxins is alpha hemolysin (Hla), which exerts cytolytic function on lymphocytes, macrophages, lung epithelial cells and pulmonary endothelial cells.

S. aureus infections, including MRSA, generally start as small red bumps that resemble pimples, boils or spider bites. These bumps or blemishes can quickly turn into deep, painful abscesses that require surgical draining. Sometimes the bacteria remain confined to the skin. On occasion, they can burrow deep into the body, causing potentially life-threatening infections in a broad range of human tissue, including skin, soft tissue, bones, joints, surgical wounds, the bloodstream, heart valves, lungs, or other organs. Thus, S. aureus infections can result in disease conditions associated therewith, which are potentially fatal diseases, such as necrotizing fasciitis, endocarditis, sepsis, bacteremia, peritonitis, toxic shock syndrome, and various forms of pneumonia, including necrotizing pneumonia, and toxin production in furunculosis and carbunculosis. MRSA infection is especially troublesome in hospital or nursing home settings where patients are at risk of or prone to open wounds, invasive devices, and weakened immune systems and, thus, are at greater risk for infection than the general public.

Antibodies neutralizing S. aureus toxins are interfering with the pathogens and pathogenic reactions, thus able to limit or prevent infection and/or to ameliorate a disease condition resulting from such infection, or to inhibit S. aureus pathogenesis, in particular pneumonia, peritonitis, osteomyelitis, bacteremia and sepsis pathogenesis. In this regard “protective antibodies” are understood herein as neutralizing antibodies that are responsible for immunity to an infectious agent observed in active or passive immunity. In particular, protective antibodies as described herein are able to neutralize toxic effects (such as cytolysis, induction of pro-inflammatory cytokine expression by target cells) of secreted virulence factors (exotoxins) and hence interfere with pathogenic potential of S. aureus.

The term “recombinant” as used herein shall mean “being prepared by or the result of genetic engineering”. A recombinant host specifically comprises an expression vector or cloning vector, or it has been genetically engineered to contain a recombinant nucleic acid sequence, in particular employing nucleotide sequence foreign to the host. A recombinant protein is produced by expressing a respective recombinant nucleic acid in a host. The term “recombinant antibody”, as used herein, includes antibodies that are prepared, expressed, created or isolated by recombinant means, such as (a) antibodies isolated from an animal (e.g., a mouse) that is transgenic or transchromosomal for human immunoglobulin genes or a hybridoma prepared therefrom, (b) antibodies isolated from a host cell transformed to express the antibody, e.g., from a transfectoma, (c) antibodies isolated from a recombinant, combinatorial human antibody library, and (d) antibodies prepared, expressed, created or isolated by any other means that involve splicing of human immunoglobulin gene sequences to other DNA sequences. Such recombinant antibodies comprise antibodies engineered to include rearrangements and mutations which occur, for example, during antibody maturation.

As used herein, the term “specificity” or “specific binding” refers to a binding reaction which is determinative of the cognate ligand of interest in a heterogeneous population of molecules. Thus, under designated conditions (e.g. immunoassay conditions), an antibody specifically binds to its particular target and does not bind in a significant amount to other molecules present in a sample. The specific binding means that binding is selective in terms of target identity, high, medium or low binding affinity or avidity, as selected. Selective binding is usually achieved if the binding constant or binding dynamics is at least 10 fold different (understood as at least 1 log difference), preferably the difference is at least 100 fold (understood as at least 2 logs difference), and more preferred a least 1000 fold (understood as at least 3 logs difference) as compared to another antigen.

The term “specificity” or “specific binding” is also understood to apply to binders which bind to one or more molecules, e.g. cross-specific binders. Preferred cross-specific (also called polyspecific or cross-reactive) binders targeting at least two different antigens or targeting a cross-reactive epitope on at least two different antigens, specifically bind the antigens with substantially similar binding affinity, e.g. with less than 100 fold difference or even less than 10 fold difference.

For example, a cross-specific antibody will be able to bind to the various antigens carrying a cross-reactive epitope. Such binding site of an antibody or and antibody with a specificity to bind at least two different antigens or a cross-reactive epitope of at least two different antigens is also called a polyspecific or cross-specific binding site and antibody, respectively. For example, an antibody may have a polyspecific binding site specifically binding an epitope cross-reactive a number of different antigens with sequence homology within the epitope and/or structural similarities to provide for a conformational epitope of essentially the same structure, e.g. cross-reactive at least the Hla and a bi-component toxin of S. aureus.

The immunospecificity of an antibody, its binding capacity and the attendant affinity the antibody exhibits for a cross-reactive binding sequence, are determined by a cross-reactive binding sequence with which the antibody immunoreacts (binds). The cross-reactive binding sequence specificity can be defined, at least in part, by the amino acid residues of the variable region of the heavy chain of the immunoglobulin the antibody and/or by the light chain variable region amino acid residue sequence.

Use of the term “having the same specificity”, “having the same binding site” or “binding the same epitope” indicates that equivalent monoclonal antibodies exhibit the same or essentially the same, i.e. similar immunoreaction (binding) characteristics and compete for binding to a pre-selected target binding sequence. The relative specificity of an antibody molecule for a particular target can be relatively determined by competition assays, e.g. as described in Harlow, et al., ANTIBODIES: A LABORATORY MANUAL, Cold Spring Harbor Laboratory Press, Cold Spring Harbor, N. Y., 1988).

The term “subject” as used herein shall refer to a warm-blooded mammalian, particularly a human being or a non-human animal. MRSA is a critically important human pathogen that is also an emerging concern in veterinary medicine. It is present in a wide range of non-human animal species. Thus, the term “subject” may also particularly refer to animals including dogs, cats, rabbits, horses, cattle, pigs and poultry. In particular the medical use of the invention or the respective method of treatment applies to a subject in need of prophylaxis or treatment of a disease condition associated with a S. aureus infection. The subject may be a patient at risk of a S: aureus infection or suffering from disease, including early stage or late stage disease. The term “patient” includes human and other mammalian subjects that receive either prophylactic or therapeutic treatment. The term “treatment” is thus meant to include both prophylactic and therapeutic treatment.

A subject is e.g. treated for prophylaxis or therapy of S. aureus disease conditions. In particular, the subject is treated, which is either at risk of infection or developing such disease or disease recurrence, or a subject that is suffering from such infection and/or disease associated with such infection.

Specifically the term “prophylaxis” refers to preventive measures which is intended to encompass prevention of the onset of pathogenesis or prophylactic measures to reduce the risk of pathogenesis.

Specifically, the method for treating, preventing, or delaying a disease condition in a subject as described herein, is by interfering with the pathogenesis of S. aureus as causal agent of the condition, wherein the pathogenesis includes a step of forming a pore on the subject's cellular membrane, e.g. by the specific virulence factors or toxins.

The term “toxin” as used herein shall refer to the alpha-toxin (Hla) and the bi-component toxins of S. aureus. It is specifically understood that the toxins targeted by the antibody of the invention are either the toxins as such, e.g. the soluble monomeric toxins or in the form of the pore forming toxins as expressed by S. aureus, or toxin components, such as the components of the bi-component toxins. Therefore, the term “toxin” as used herein shall refer to both, the toxin or the toxin components bearing the immunorelevant epitope.

The virulence of S. aureus is due to a combination of numerous virulence factors, which include surface-associated proteins that allow the bacterium to adhere to eukaryotic cell membranes, a capsular polysaccharide that protects it from opsonophagocytosis, and several exotoxins. S. aureus causes disease mainly through the production of secreted virulence factors such as hemolysins, enterotoxins and toxic shock syndrome toxin. These secreted virulence factors suppress the immune response by inactivating many immunological mechanisms in the host, and cause tissue destruction and help establish the infection. The latter is accomplished by a group of pore forming toxins, the most prominent of which is Hla, a key virulence factor for S. aureus pneumonia.

S. aureus produces a diverse array of further virulence factors and toxins that enable this bacterium to counteract and withstand attack by different kinds of immune cells, specifically subpopulations of white blood cells that make up the body's primary defense system. The production of these virulence factors and toxins allow S. aureus to maintain an infectious state. Among these virulence factors, S. aureus produces several bi-component leukotoxins, which damage membranes of host defense cells and erythrocytes by the synergistic action of two non-associated proteins or subunits. Among these bi-component toxins, gamma-hemolysin (HlgAB and HlgCB) and the Pantone-Valentine Leukocidin (PVL) are the best characterized.

The toxicity of the leukocidins towards mammalian cells involves the action of two components. The first subunit is named class S component, and the second subunit is named class F component. The S and F subunits act synergistically to form pores on white blood cells including monocytes, macrophages, dendritic cells and neutrophils (collectively known as phagocytes). The gamma hemolysins, especially HlgAB and HlgA-LukD also act on red blood cells and LukED on T cells. The repertoire of bi-component leukotoxins produced by S. aureus is known to include cognate and non-cognate pairs of the F and S components, e.g. gamma-hemolysins, PVL toxins and PVL-like toxins, including HlgAB, HlgCB, LukSF, LukED, LukGH, LukS-HlgB, LukSD, HlgA-LukD, HlgA-LukF, LukG-HlgA, LukEF, LukE-HlgB, HlgC-LukD or HlgC-LukF, which are preferred targets as described herein.

The term “substantially pure” or “purified” as used herein shall refer to a preparation comprising at least 50% (w/w), preferably at least 60%, 70%, 80%, 90% or 95% of a compound, such as a nucleic acid molecule or an antibody. Purity is measured by methods appropriate for the compound (e.g. chromatographic methods, polyacrylamide gel electrophoresis, HPLC analysis, and the like).

The term “therapeutically effective amount”, used herein interchangeably with any of the terms “effective amount” or “sufficient amount” of a compound, e.g. an antibody or immunogen of the present invention, is a quantity or activity sufficient to, when administered to the subject effect beneficial or desired results, including clinical results, and, as such, an effective amount or synonym thereof depends upon the context in which it is being applied.

An effective amount is intended to mean that amount of a compound that is sufficient to treat, prevent or inhibit such diseases or disorder. In the context of disease, therapeutically effective amounts of the antibody as described herein are specifically used to treat, modulate, attenuate, reverse, or affect a disease or condition that benefits from an inhibition of S. aureus or S. aureus pathogenesis.

The amount of the compound that will correspond to such an effective amount will vary depending on various factors, such as the given drug or compound, the pharmaceutical formulation, the route of administration, the type of disease or disorder, the identity of the subject or host being treated, and the like, but can nevertheless be routinely determined by one skilled in the art.

The antibody or the immunogen of the present invention may be used prophylactically to inhibit onset of S. aureus infection, or therapeutically to treat S. aureus infection, particularly S. aureus infections such as MRSA that are known to be refractory or in the case of the specific subject, have proven refractory to treatment with other conventional antibiotic therapy.

A therapeutically effective amount of the antibody as described herein, such as provided to a human patient in need thereof, may specifically be in the range of 0.5-50 mg/kg, preferably 5-40 mg/kg, even more preferred up to 20 mg/kg, up to 10 mg/kg, up to 5 mg/kg, though higher doses may be indicated e.g. for treating acute disease conditions.

Moreover, a treatment or prevention regime of a subject with a therapeutically effective amount of the antibody of the present invention may consist of a single administration, or alternatively comprise a series of applications. For example, the antibody may be administered at least once a year, at least once a half-year or at least once a month. However, in another embodiment, the antibody may be administered to the subject from about one time per week to about a daily administration for a given treatment. The length of the treatment period depends on a variety of factors, such as the severity of the disease, either acute or chronic disease, the age of the patient, the concentration and the activity of the antibody format. It will also be appreciated that the effective dosage used for the treatment or prophylaxis may increase or decrease over the course of a particular treatment or prophylaxis regime. Changes in dosage may result and become apparent by standard diagnostic assays known in the art. In some instances, chronic administration may be required.

An effective amount of an immunogen as described herein, such as provided to a patient at risk of developing a disease condition associated with an S. aureus infection, may specifically be in the range of 1-20 mg/kg per dose.

For example, the immunogen may be administered as a first dose followed by one or more booster dose(s), within a certain timeframe, according to a prime-boost immunization scheme to induce a long-lasting, efficacious immune response to S. aureus infection. A preferred vaccination schedule would encompass administration of three doses, e.g. a first dose on day 0, a second dose on day 5-40, and a third dose on day 10-100, preferably on days 0, 28 and 90. According to a preferred accelerated schedule the administration may be on days 0, 7 and 14. Accelerated schedules may be indicated for prophylaxis, e.g. for patients facing elective surgery. Usually alum is used as an adjuvant, e.g. as phosphate or hydroxide.

Therefore, the invention specifically refers to antibodies cross-neutralizing both alpha hemolysin and bi-component toxins of S. aureus. This was surprising, because of the low level of sequence homology. The chance to generate mAbs cross-neutralizing Hla and at least one bi-component toxin was expected to be low. Such cross-neutralizing antibodies are of great potential value.

The only publication describing multiple bi-component specificity antibodies (Laventie, PNAS, 2011, 108:16404) is considered to be non-relevant for the current invention, since it was designed to target LukS and HlgC only.

The present invention specifically refers to an antibody that targets several toxins by the same binding site, herein referred to as a polyspecific binding site, which is able to bind to the different toxins, e.g. four different toxins (quadriple reactive), which are alpha-toxin and F-components of the gamma-hemolysin, the Panten Valentine leukocidin (PVL, LukSF) and LukED. It is feasible that the quadriple reactive mAb also binds the bovine LukM leukocidin based on high amino acid homology to LukED and LukSF.

In some embodiments, the antibodies of the invention that recognize an epitope on Hla and cross-react with HlgB, may have additionally cross-reactivity towards other staphylococcal leukocidin F compounds such as LukF′-PV, LukF-PV, LukDv, LukD, LukF-I, and LukG. Cross-reactive anti-HlgB antibodies of the invention may inhibit or reduce HlgB activity. In some embodiments, the cross-reactive anti-HlgB antibodies neutralize, e.g. substantially eliminate HlgB activity.

According to a specific aspect, there is provided an antibody binding the same epitope, which term includes variants binding to essentially the same epitope, as the parent antibody which is characterized by the polyspecific binding site formed by the VH amino acid sequence of SEQ ID 20 and the VL amino acid sequence of SEQ ID 39, or else by the HC amino acid sequence of SEQ ID 40 and the LC amino acid sequence of SEQ ID 52. Such antibodies may e.g. be functionally active variant antibodies obtained by modifying the respective CDR or antibody sequence of the parent antibody.

Exemplary parent antibodies are described in the examples section below and in FIG. 1. The antibody designated #AB-28 is e.g. used as a parent antibody to produce functionally active CDR variants with one or more modified CDR sequences, and functionally active antibody variants with one or more modified FR sequences, such as sequences of FR1, FR2, FR3 or FR4, or a constant domain sequence, and/or with one or more modified CDR sequences. The variant antibody derived from the parent antibody by mutagenesis are exemplified below and designated #AB-28-3, #AB-28-4, #AB-28-5, #AB-28-6, #AB-28-7, #AB-28-8, #AB-28-9, #AB-28-10, #AB-28-11, #AB-28-12, or #AB-28-13 (see FIG. 1). Though these variant antibodies share the common VL sequence of SEQ ID39, it is feasible that also variant VL chains, e.g. with modifications in the respective FR or CDR sequences may be used, which are functionally active.

Further antibody variants are feasible, which comprise the same binding site, which term includes variants comprising essentially the same binding site, as the antibody designated #AB-28. The #AB-28 antibody and functionally active variants would particularly comprise a binding site potently neutralizing Hla and cross-neutralizing at least one of, at least two of or at least the three cognate toxin pairs LukS-LukF, LukE-LukD, and HlgB-HlgC, and possibly further bi-component toxins.

Specifically, there is provided an antibody comprising the variable region of the antibody designated #AB-28, in particular at least one of the CDR sequences, preferably at least two, at least 3, at least 4, at least 5 or at least six of the CDR sequences, or CDR variants thereof which are functionally active. More specifically, there is provided the antibody designated #AB-28, #AB-28-3, #AB-28-4, #AB-28-5, #AB-28-6, #AB-28-7, #AB-28-8, #AB-28-9, #AB-28-10, #AB-28-11, #AB-28-12, or #AB-28-13.

Specifically, the #AB-28 antibody or any functionally active variant thereof may be produced employing the sequences as provided herein by recombinant means, optionally with further immunoglobulin sequences, e.g. to produce an IgG antibody.

In certain aspects, the invention provides for such functionally active variant antibodies, preferably monoclonal antibodies, most preferably human antibodies, comprising a heavy chain and a light chain, wherein any of the heavy chain or VH variable region or the respective CDRs comprises an amino acid sequence as derived from a parent antibody, which is one of the #AB-28, #AB-28-3, #AB-28-4, #AB-28-5, #AB-28-6, #AB-28-7, #AB-28-8, #AB-28-9, #AB-28-10, #AB-28-11, #AB-28-12, or #AB-28-13 antibodies, by modification of at least one FR or CDR sequences.

In certain aspects, the invention provides for such functionally active variant antibodies, preferably monoclonal antibodies, most preferably human antibodies, comprising a heavy chain and a light chain, wherein any of the light chain or VL variable region or the respective CDRs comprises an amino acid sequence as derived from a parent antibody, which is the #AB-28 antibody, by modification of at least one FR or CDR sequences.

In certain aspects, the invention provides for such variant antibodies, preferably monoclonal antibodies, most preferably human antibodies, comprising a heavy chain and a light chain, wherein any of the heavy and light chain, or the VHNL variable regions, or the respective CDRs comprises an amino acid sequence as derived from a parent antibody, which is one of the #AB-28, #AB-28-3, #AB-28-4, #AB-28-5, #AB-28-6, #AB-28-7, #AB-28-8, #AB-28-9, #AB-28-10, #AB-28-11, #AB-28-12, or #AB-28-13 antibodies, by modification of at least one FR or CDR sequences.

In certain aspects, the invention also provides for such variant antibodies, comprising the respective binding sequences, such as the variable sequences and/or the CDR sequences, as derived from the parent antibodies above, wherein the binding sequences or the CDR comprises a sequence that has at least 60%, preferably at least 70%, or at least 80%, or at least 90%, or at least 95%, or at least 99% identity to the amino acid sequence as derived from the parent antibodies, and wherein the variant is a functionally active variant.

In particular, the functional activity is determined by the cross-reactivity to target the specific toxins, e.g. by binding the same epitope or substantially the same epitope as the respective parent antibody.

Antibodies are said to “bind to the same epitope” or “comprising the same binding site” or have “essentially the same binding” characteristics, if the antibodies cross-compete so that only one antibody can bind to the epitope at a given point of time, i.e. one antibody prevents the binding or modulating effect of the other.

The term “compete” or “cross-compete”, as used herein with regard to an antibody, means that a first antibody, or an antigen-binding portion thereof, binds to an epitope in a manner sufficiently similar to the binding of a second antibody, or an antigen-binding portion thereof, such that the result of binding of the first antibody with its cognate epitope is detectably decreased in the presence of the second antibody compared to the binding of the first antibody in the absence of the second antibody. The alternative, where the binding of the second antibody to its epitope is also detectably decreased in the presence of the first antibody, can, but need not be the case. That is, a first antibody can inhibit the binding of a second antibody to its epitope without that second antibody inhibiting the binding of the first antibody to its respective epitope. However, where each antibody detectably inhibits the binding of the other antibody with its cognate epitope, whether to the same, greater, or lesser extent, the antibodies are said to “cross-compete” with each other for binding of their respective epitope(s). Both competing and cross-competing antibodies are encompassed by the present invention.

Competition herein means a greater relative inhibition than about 30% as determined by competition ELISA analysis or by ForteBio analysis, e.g. as described in the Examples section. It may be desirable to set a higher threshold of relative inhibition as criteria of what is a suitable level of competition in a particular context, e.g., where the competition analysis is used to select or screen for new antibodies designed with the intended function of the binding of additional or other toxins of S. aureus. Thus, for example, it is possible to set criteria for the competitive binding, wherein at least 40% relative inhibition is detected, or at least 50%, at least 60%, at least 70%, at least 80%, at least 90% or even at least 100%, before an antibody is considered sufficiently competitive.

As described herein, in one aspect the invention provides antibody molecules characterized by, e.g. the ability to compete with any of the #AB-28, #AB-28-3, #AB-28-4, #AB-28-5, #AB-28-6, #AB-28-7, #AB-28-8, #AB-28-9, #AB-28-10, #AB-28-11, #AB-28-12, or #AB-28-13 antibodies for binding to any of Hla, LukSF, LukED and HlgCB, or binding to each of Hla, LukF, LukD and HlgB.

Preferred antibodies of the invention are binding said individual antigens with a high affinity, in particular with a high on and/or a low off rate, or a high avidity of binding. The binding affinity of an antibody is usually characterized in terms of the concentration of the antibody, at which half of the antigen binding sites are occupied, known as the dissociation constant (Kd, or K_{D}). Usually a binder is considered a high affinity binder with a Kd<10⁻⁸ M, preferably a Kd<10⁻⁹ M, even more preferred is a Kd<10⁻¹⁹ M.

Yet, in a particularly preferred embodiment the individual antigen binding affinities are of medium affinity, e.g. with a Kd of less than 10⁻⁶ and up to 10⁻⁸ M, e.g. when binding to at least two antigens.

Medium affinity binders may be provided according to the invention, preferably in conjunction with an affinity maturation process, if necessary.

Affinity maturation is the process by which antibodies with increased affinity for a target antigen are produced. Any one or more methods of preparing and/or using affinity maturation libraries available in the art may be employed in order to generate affinity matured antibodies in accordance with various embodiments of the invention disclosed herein. Exemplary such affinity maturation methods and uses, such as random mutagenesis, bacterial mutator strains passaging, site-directed mutagenesis, mutational hotspots targeting, parsimonious mutagenesis, antibody shuffling, light chain shuffling, heavy chain shuffling, CDR1 and/or CDR1 mutagenesis, and methods of producing and using affinity maturation libraries amenable to implementing methods and uses in accordance with various embodiments of the invention disclosed herein, include, for example, those disclosed in: Prassler et al. (2009); Immunotherapy, Vol. 1(4), pp. 571-583; Sheedy et al. (2007), Biotechnol. Adv., Vol. 25(4), pp. 333-352; WO2012/009568; WO2009/036379; WO2010/105256; US2002/0177170; WO2003/074679.

With structural changes of an antibody, including amino acid mutagenesis or as a consequence of somatic mutation in immunoglobulin gene segments, variants of a binding site to an antigen are produced and selected for greater affinities. Affinity matured antibodies may exhibit a several logfold greater affinity than a parent antibody. Single parent antibodies may be subject to affinity maturation. Alternatively pools of antibodies with similar binding affinity to the target antigen may be considered as parent structures that are varied to obtain affinity matured single antibodies or affinity matured pools of such antibodies.

The preferred affinity maturated variant of an antibody according to the invention exhibits at least a 2 fold increase in affinity of binding, preferably at least a 5, preferably at least 10, preferably at least 50, or preferably at least 100 fold increase. The affinity maturation may be employed in the course of the selection campaigns employing respective libraries of parent molecules, either with antibodies having medium binding affinity to obtain the antibody of the invention having the specific target binding property of a binding affinity Kd<10⁻⁸ M. Alternatively, the affinity may be even more increased by affinity maturation of the antibody according to the invention to obtain the high values corresponding to a Kd of less than 10⁻⁹ M, preferably less than 10⁻¹⁹ M or even less than 10⁻¹¹ M, most preferred in the picomolar range.

In certain embodiments binding affinity is determined by an affinity ELISA assay. In certain embodiments binding affinity is determined by a BIAcore, ForteBio or MSD assays. In certain embodiments binding affinity is determined by a kinetic method. In certain embodiments binding affinity is determined by an equilibrium/solution method.

Phagocytic effector cells may be activated through another route employing activation of complement. Antibodies that bind to surface antigens on microorganisms attract the first component of the complement cascade with their Fc region and initiate activation of the “classical” complement system. These results in the stimulation of phagocytic effector cells, which ultimately kill the target by complement dependent cytotoxicity (CDC).

According to a specific embodiment, the antibody of the invention has a cytotoxic activity in the presence of immune-effector cells as measured in a standard ADCC or CDC assay. A cytotoxic activity as determined by either of an ADCC or CDC assay may be shown for an antibody of the invention, if there is a significant increase in the percentage of cytolysis as compared to a control. The cytotoxic activity related to ADCC or CDC is preferably measured as the absolute percentage increase, which is preferably higher than 5%, more preferably higher than 10%, even more preferred higher than 20%. Complement fixation might be specifically relevant, this mechanism can eliminate toxins from the infection site or blood by removal of the immune complexes formed.

According to a specific embodiment, the antibody of the invention has an immunomodulatory function exerted by the Fc part of IgGs. Altered glycosylation increasing the sialylation content, e.g. on the terminal galactose residues, possibly have an anti-inflammatory effect via DC-SIGN signaling. Preferential binding to Fcgamma receptor IIb (inhibitory) over the Ia, IIa and III Fcgamma receptors possibly provides an anti-inflammatory effect.

The invention specifically provides for cross-reactive antibodies, which are obtained by a process to identify neutralizing antibodies with multiple specificities, e.g. by a cross-reactive discovery selection scheme. Accordingly, an antibody library including antibodies showing reactivity with two targets, targets A and B, may first be selected for reactivity with one of the targets, e.g. target A, followed by selection for reactivity with the other target, e.g. target B. Each successive selection round reinforces the reactive strength of the resulting pool towards both targets. Accordingly, this method is particularly useful for identifying antibodies with cross-reactivity directed to the two different targets, and the potential to cross-neutralize a pathogen. The method can be extended to identifying antibodies showing reactivity towards further targets, by including additional rounds of enrichment towards the additional target(s).

Cross-reactive antibodies, in some instances, emerge through screening against single antigens. To increase the likelihood of isolating cross-reactivity clones one would apply multiple selective pressures by processively screening against multiple antigens. Special mAb selection strategies employ the different toxin components in an alternating fashion. For example, neutralizing anti-Hla mAbs are tested for binding to PVL and PVL like toxins on human neutrophils, which represent the major target for bi-component toxins during S. aureus infection.

The recombinant toxins produced by recombinant techniques employing the respective sequences of FIG. 7, or toxins isolated from S. aureus culture supernatants may be used for selecting antibodies from an antibody library, e.g. a yeast-displayed antibody library see, for example: Blaise L, Wehnert A, Steukers M P, van den Beucken T, Hoogenboom H R, Hufton S E. Construction and diversification of yeast cell surface displayed libraries by yeast mating: application to the affinity maturation of Fab antibody fragments. Gene. 2004 Nov. 24; 342(2):211-8; Boder E T, Wittrup K D. Yeast surface display for screening combinatorial polypeptide libraries. Nat Biotechnol. 1997 June; 15(6):553-7; Kuroda K, Ueda M. Cell surface engineering of yeast for applications in white biotechnology. Biotechnol Lett. 2011 January; 33(1):1-9. doi: 10.1007/s10529-010-0403-9. Review; Lauer T M, Agrawal N J, Chennamsetty N, Egodage K, Helk B, Trout B L. Developability index: a rapid in silico tool for the screening of antibody aggregation propensity. J Pharm Sci. 2012 January; 101(1):102-15; Orcutt K. D. and Wittrup K. D. (2010), 207-233 doi: 10.1007/978-3-642-01144-3_15; Rakestraw J A, Aird D, Aha P M, Baynes B M, Lipovsek D. Secretion-and-capture cell-surface display for selection of target-binding proteins. Protein Eng Des Sel. 2011 June; 24(6):525-30; U.S. Pat. No. 6,423,538; U.S. Pat. No. 6,696,251; U.S. Pat. No. 6,699,658; published PCT application publication No. WO2008118476.

In either event cross-reactivity can be further improved by antibody optimization methods known in the art. For example, certain regions of the variable regions of the immunoglobulin chains described herein may be subjected to one or more optimization strategies, including light chain shuffling, destinational mutagenesis, CDR amalgamation, and directed mutagenesis of selected CDR and/or framework regions.

Screening methods for identifying antibodies with the desired neutralizing properties may be inhibition of toxin binding to the target cells, inhibition of formation of dimers or oligomers, inhibition of pore formation, inhibition of cell lysis, inhibition of the induction of cytokines, lymphokines, and any pro-inflammatory signaling, and/or inhibition of in vivo effect on animals (death, hemolysis, overshooting inflammation, organ dysfunction). Reactivity can be assessed based on direct binding to the desired toxins, e.g. using standard assays.

Once cross-neutralizing antibodies with the desired properties have been identified, the dominant epitope or epitopes recognized by the antibodies may be determined. Methods for epitope mapping are well-known in the art and are disclosed, for example, in Epitope Mapping: A Practical Approach, Westwood and Hay, eds., Oxford University Press, 2001.

Epitope mapping concerns the identification of the epitope to which an antibody binds. There are many methods known to those of skill in the art for determining the location of epitopes on proteins, including crystallography analysis of the antibody-antigen complex, competition assays, gene fragment expression assays, and synthetic peptide-based assays. An antibody that “binds the same epitope” as a reference antibody is herein understood in the following way. When two antibodies recognize epitopes that are identical or sterically overlapping epitopes, the antibodies are referred to as binding the same or essentially the same or substantially the same epitopes. A commonly used method for determining whether two antibodies bind to identical or sterically overlapping epitopes is the competition assay, which can be con-figured in all number of different formats, using either labeled antigen or labeled antibody. Usually, an antigen is immobilized on a 96-well plate, and the ability of unlabeled antibodies to block the binding of labeled antibodies is measured using radioactive or enzyme labels.

Once antibodies with the desired cross-neutralizing properties are identified, such antibodies, including antibody fragments can be produced by methods well-known in the art, including, for example, hybridoma techniques or recombinant DNA technology.

In the hybridoma method, a mouse or other appropriate host animal, such as a hamster, is immunized to elicit lymphocytes that produce or are capable of producing antibodies that will specifically bind to the protein used for immunization. Alternatively, lymphocytes may be immunized in vitro. Lymphocytes then are fused with myeloma cells using a suitable fusing agent, such as polyethylene glycol, to form a hybridoma cell.

Culture medium in which hybridoma cells are growing is assayed for production of monoclonal antibodies directed against the antigen. Preferably, the binding specificity of monoclonal antibodies produced by hybridoma cells is determined by immunoprecipitation or by an in vitro binding assay, such as radioimmunoassay (RIA) or enzyme-linked immunoabsorbent assay (ELISA).

Recombinant monoclonal antibodies can, for example, be produced by isolating the DNA encoding the required antibody chains and transfecting a recombinant host cell with the coding sequences for expression, using well known recombinant expression vectors, e.g. the plasmids of the invention or expression cassette(s) comprising the nucleotide sequences encoding the antibody sequences. Recombinant host cells can be prokaryotic and eukaryotic cells, such as those described above.

According to a specific aspect, the nucleotide sequence may be used for genetic manipulation to humanize the antibody or to improve the affinity, or other characteristics of the antibody. For example, the constant region may be engineered to more nearly resemble human constant regions to avoid immune response, if the antibody is used in clinical trials and treatments in humans. It may be desirable to genetically manipulate the antibody sequence to obtain greater affinity to the target toxins and greater efficacy against S. aureus. It will be apparent to one of skill in the art that one or more polynucleotide changes can be made to the antibody and still maintain its binding ability to the target toxins.

The production of antibody molecules, by various means, is generally well understood. U.S. Pat. No. 6,331,415 (Cabilly et al.), for example, describes a method for the recombinant production of antibodies where the heavy and light chains are expressed simultaneously from a single vector or from two separate vectors in a single cell. Wibbenmeyer et al., (1999, Biochim Biophys Acta 1430(2):191-202) and Lee and Kwak (2003, J. Biotechnology 101:189-198) describe the production of monoclonal antibodies from separately produced heavy and light chains, using plasmids expressed in separate cultures of E. coli. Various other techniques relevant to the production of antibodies are provided in, e.g., Harlow, et al., ANTIBODIES: A LABORATORY MANUAL, Cold Spring Harbor Laboratory Press, Cold Spring Harbor, N.Y., (1988).

If desired, the antibody of the invention, e.g. any of the #AB-28, #AB-28-3, #AB-28-4, #AB-28-5, #AB-28-6, #AB-28-7, #AB-28-8, #AB-28-9, #AB-28-10, #AB-28-11, #AB-28-12, or #AB-28-13 antibodies, may be sequenced and the polynucleotide sequence may then be cloned into a vector for expression or propagation. The sequence encoding the antibody may be maintained in vector in a host cell and the host cell can then be expanded and frozen for future use. Production of recombinant monoclonal antibodies in cell culture can be carried out through cloning of antibody genes from B cells by means known in the art.

In another aspect, the invention provides an isolated nucleic acid comprising a sequence that codes for production of the recombinant antibody of the present invention.

In another aspect, the invention provides an isolated nucleic acid comprising a sequence that codes for production of the recombinant epitope of the present invention, or a molecule comprising such epitope of the present invention. However, the epitope of the invention may also be synthetically produced, e.g. through any of the synthesis methods well-known in the art.

An antibody or epitope encoding nucleic acid can have any suitable characteristics and comprise any suitable features or combinations thereof. Thus, for example, an antibody or epitope encoding nucleic acid may be in the form of DNA, RNA, or a hybrid thereof, and may include non-naturally-occurring bases, a modified backbone, e.g., a phosphorothioate backbone that promotes stability of the nucleic acid, or both. The nucleic acid advantageously may be incorporated in an expression cassette, vector or plasmid of the invention, comprising features that promote desired expression, replication, and/or selection in target host cell(s). Examples of such features include an origin of replication component, a selection gene component, a promoter component, an enhancer element component, a polyadenylation sequence component, a termination component, and the like, numerous suitable examples of which are known.

The present disclosure further provides the recombinant DNA constructs comprising one or more of the nucleotide sequences described herein. These recombinant constructs are used in connection with a vector, such as a plasmid, phagemid, phage or viral vector, into which a DNA molecule encoding any disclosed antibody is inserted.

Monoclonal antibodies are produced using any method that produces antibody molecules by continuous cell lines in culture. Examples of suitable methods for preparing monoclonal antibodies include the hybridoma methods of Kohler et al. (1975, Nature 256:495-497) and the human B-cell hybridoma method (Kozbor, 1984, J. Immunol. 133:3001; and Brodeur et al., 1987, Monoclonal Antibody Production Techniques and Applications, (Marcel Dekker, Inc., New York), pp. 51-63).

The invention moreover provides pharmaceutical compositions which comprise an antibody or an immunogen as described herein and a pharmaceutically acceptable carrier or excipient. These pharmaceutical compositions can be administered in accordance with the present invention as a bolus injection or infusion or by continuous infusion. Pharmaceutical carriers suitable for facilitating such means of administration are well known in the art.

Pharmaceutically acceptable carriers generally include any and all suitable solvents, dispersion media, coatings, antibacterial and antifungal agents, isotonic and absorption delaying agents, and the like that are physiologically compatible with an antibody or related composition or combination provided by the invention. Further examples of pharmaceutically acceptable carriers include sterile water, saline, phosphate buffered saline, dextrose, glycerol, ethanol, and the like, as well as combinations of any thereof.

In one such aspect, an antibody can be combined with one or more carriers appropriate a desired route of administration, antibodies may be, e.g. admixed with any of lactose, sucrose, starch, cellulose esters of alkanoic acids, stearic acid, talc, magnesium stearate, magnesium oxide, sodium and calcium salts of phosphoric and sulphuric acids, acacia, gelatin, sodium alginate, polyvinylpyrrolidine, polyvinyl alcohol, and optionally further tabletted or encapsulated for conventional administration. Alternatively, an antibody may be dissolved in saline, water, polyethylene glycol, propylene glycol, carboxymethyl cellulose colloidal solutions, ethanol, corn oil, peanut oil, cotton-seed oil, sesame oil, tragacanth gum, and/or various buffers. Other carriers, adjuvants, and modes of administration are well known in the pharmaceutical arts. A carrier may include a controlled release material or time delay material, such as glyceryl monostearate or glyceryl distearate alone or with a wax, or other materials well known in the art.

Additional pharmaceutically acceptable carriers are known in the art and described in, e.g. REMINGTON'S PHARMACEUTICAL SCIENCES. Liquid formulations can be solutions, emulsions or suspensions and can include excipients such as suspending agents, solubilizers, surfactants, preservatives, and chelating agents.

Pharmaceutical compositions are contemplated wherein an antibody or immunogen of the present invention and one or more therapeutically active agents are formulated. Stable formulations of the antibody or immunogen of the present invention are prepared for storage by mixing said immunoglobulin having the desired degree of purity with optional pharmaceutically acceptable carriers, excipients or stabilizers, in the form of lyophilized formulations or aqueous solutions. The formulations to be used for in vivo administration are specifically sterile, preferably in the form of a sterile aqueous solution. This is readily accomplished by filtration through sterile filtration membranes or other methods. The antibody and other therapeutically active agents disclosed herein may also be formulated as immunoliposomes, and/or entrapped in microcapsules.

Administration of the pharmaceutical composition comprising an antibody or immunogen of the present invention, may be done in a variety of ways, including orally, subcutaneously, intravenously, intranasally, intraotically, transdermally, mucosal, topically, e.g., gels, salves, lotions, creams, etc., intraperitoneally, intramuscularly, intrapulmonary, e.g. employing inhalable technology or pulmonary delivery systems, vaginally, parenterally, rectally, or intraocularly.

Exemplary formulations as used for parenteral administration include those suitable for subcutaneous, intramuscular or intravenous injection as, for example, a sterile solution, emulsion or suspension.

In one embodiment, the antibody or immunogen of the present invention is the only therapeutically active agent administered to a subject, e.g. as a disease modifying or preventing monotherapy.

In another embodiment, the antibody or immunogen of the present invention is combined with further antibodies or immunogens in a cocktail, e.g. combined in a mixture or kit of parts, to target S. aureus, such that the cocktail contains more than one therapeutically active agents administered to a subject, e.g. as a disease modifying or preventing combination therapy.

Alternatively, the antibody or immunogen of the present invention is administered in combination with one or more other therapeutic or prophylactic agents, including but not limited to standard treatment, e.g. antibiotics, steroid and non-steroid inhibitors of inflammation, and/or other antibody based therapy, e.g. employing antibacterial or anti-inflammatory agents.

A combination therapy is particularly employing a standard regimen, e.g. as used for treating MRSA infection. This may include antibiotics, e.g. tygecycline, linezolide, methicillin and/or vancomycin.

In a combination therapy, the antibody may be administered as a mixture, or concomitantly with one or more other therapeutic regimens, e.g. either before, simultaneously or after concomitant therapy.

Prophylactic administration of immunogens in some cases may employ a vaccine comprising the immunogen of the present invention, i.e. a monovalent vaccine. Yet, a multivalent vaccine comprising different immunogens to induce an immune response against the same or different target pathogens may be used.

The biological properties of the antibody, the immunogen or the respective pharmaceutical preparations of the invention may be characterized ex vivo in cell, tissue, and whole organism experiments. As is known in the art, drugs are often tested in vivo in animals, including but not limited to mice, rats, rabbits, dogs, cats, pigs, and monkeys, in order to measure a drug's efficacy for treatment against a disease or disease model, or to measure a drug's pharmacokinetics, pharmacodynamics, toxicity, and other properties. The animals may be referred to as disease models. Therapeutics are often tested in mice, including but not limited to nude mice, SCID mice, xenograft mice, and transgenic mice (including knockins and knockouts). Such experimentation may provide meaningful data for determination of the potential of the antibody to be used as a therapeutic or as a prophylactic with the appropriate half-life, effector function, (cross-) neutralizing activity and/or immune response upon active or passive immunotherapy. Any organism, preferably mammals, may be used for testing. For example because of their genetic similarity to humans, primates, monkeys can be suitable therapeutic models, and thus may be used to test the efficacy, toxicity, pharmacokinetics, pharmacodynamics, half-life, or other property of the subject agent or composition. Tests in humans are ultimately required for approval as drugs, and thus of course these experiments are contemplated. Thus, the antibody, immunogen and respective pharmaceutical compositions of the present invention may be tested in humans to determine their therapeutic or prophylactic efficacy, toxicity, immuno-genicity, pharmacokinetics, and/or other clinical properties.

The invention also provides the subject antibody of the invention for diagnostic purposes, e.g. for use in methods of detecting and quantitatively determining the concentration of a toxin or antibody as immunoreagent or target in a biological fluid sample.

The invention also provides methods for detecting the level of toxins or S. aureus infection in a biological sample, such as a body fluid, comprising the step of contacting the sample with an antibody of the invention. The antibody of the invention may be employed in any known assay method, such as competitive binding assays, direct and indirect sandwich assays, immunoprecipitation assays and enzyme-linked immunosorbent assays (ELISA).

A body fluid as used according to the present invention includes biological samples of a subject, such as tissue extract, urine, blood, serum, stool and phlegm.

In one embodiment the method comprises contacting a solid support with an excess of a certain type of antibody fragment which specifically forms a complex with a target, such as at least one of the toxins targeted by the antibody of the invention, conditions permitting the antibody to attach to the surface of the solid support. The resulting solid support to which the antibody is attached is then contacted with a biological fluid sample so that the target in the biological fluid binds to the antibody and forms a target-antibody complex. The complex can be labeled with a detectable marker. Alternatively, either the target or the antibody can be labeled before the formation the complex. For example, a detectable marker (label) can be conjugated to the antibody. The complex then can be detected and quantitatively determined thereby detecting and quantitatively determining the concentration of the target in the biological fluid sample.

For particular applications the antibody of the invention is conjugated to a label or reporter molecule, selected from the group consisting of organic molecules, enzyme labels, radioactive labels, colored labels, fluorescent labels, chromogenic labels, luminescent labels, haptens, digoxigenin, biotin, metal complexes, metals, colloidal gold and mixtures thereof. Antibodies conjugated to labels or reporter molecules may be used, for instance, in assay systems or diagnostic methods, e.g. to diagnose S. aureus infection or disease conditions associated therewith.

The antibody of the invention may be conjugated to other molecules which allow the simple detection of said conjugate in, for instance, binding assays (e.g. ELISA) and binding studies.

Another aspect of the present invention provides a kit comprising an antibody, which may include, in addition to one or more antibodies, various diagnostic or therapeutic agents. A kit may also include instructions for use in a diagnostic or therapeutic method. Such instructions can be, for example, provided on a device included in the kit, e.g. tools or a device to prepare a biological sample for diagnostic purposes, such as separating a cell and/or protein containing fraction before determining the respective toxin(s) to diagnose a disease. Advantageously, such a kit includes an antibody and a diagnostic agent or reagent that can be used in one or more of the various diagnostic methods described herein. In another preferred embodiment, the kit includes an antibody, e.g. in the lyophilized form, in combination with pharmaceutically acceptable carrier(s) that can be mixed before use to form an injectable composition for near term administration.

The antibody designated #AB-28 specifically is characterized by amino acid sequences as indicated in FIG. 1, specifically the VH sequence of SEQ ID 20 and the HC sequence of SEQ ID 40, respectively, in particular by the VH CDR sequences CDR1 of SEQ ID 1, CDR2 of SEQ ID 2, and CDR3 of SEQ ID 3, and further characterized by the VH FR sequences FR1 of SEQ ID 13, FR2 of SEQ ID 15, FR3 of SEQ ID 17, and FR4 of SEQ ID 19, and further characterized by the VL sequence of SEQ ID 39 and the LC sequence of SEQ ID 52, respectively, in particular by the VL CDR sequences CDR4 of SEQ ID 32, the CDR5 of SEQ ID 33, and the CDR6 of SEQ ID 34, and further characterized by the VL FR sequences FR1 of SEQ ID 35, FR2 of SEQ ID 36, FR3 of SEQ ID 37, and FR4 of SEQ ID 38.

The antibody variants designated #AB-28-3, #AB-28-4, #AB-28-5, #AB-28-6, #AB-28-7, #AB-28-8, #AB-28-9, #AB-28-10, #AB-28-11, #AB-28-12, or #AB-28-13, are characterized by amino acid sequences as indicated in FIG. 1, specifically the respective VH or HC sequences, and further the VL or LC sequences, including the FR and CDR sequences as described in FIG. 1.

The antibody designated #AB-24, specifically the antibody light chain and/or heavy chain, is characterized by the biological material deposited at the DSMZ-Deutsche Sammlung von Mikroorganismen und Zellkulturen, Mascheroder Weg 1 b/Inhoffenstraβe 7B, 38124 Braunschweig (DE) under the accession numbers as indicated herein.

DSM 26747 is an E. coli host cell transformed with a plasmid comprising the coding sequence of the #AB-24 heavy chain (AB-24-HC): Escherichia coli DHSalpha AB-24-HC=DSM 26747, deposition date: Jan. 8, 2013; depositor: Arsanis Biosciences GmbH, Vienna, Austria.

DSM 26748 is an E. coli host cell transformed with a plasmid comprising the coding sequence of the #AB-24 light chain (AB-24-LC): Escherichia coli DHSalpha AB-24-LC=DSM 26748; deposition date: Jan. 8, 2013; depositor: Arsanis Biosciences GmbH, Vienna, Austria.

The subject matter of the following definitions is considered embodiments of the present invention:

1. A cross-neutralizing antibody comprising at least one polyspecific binding site that binds to alpha-toxin (Hla) and at least one of the bi-component toxins of Staphylococcus aureus, which antibody comprises at least three complementarity determining regions (CDR1 to CDR3) of the antibody heavy chain variable region (VH), wherein

A) the antibody comprises
a) a CDR1 comprising or consisting of the amino acid sequence YSISSGMGWG (SEQ ID 1); and
b) a CDR2 comprising or consisting of the amino acid sequence SIDQRGSTYYNPSLKS (SEQ ID 2); and
c) a CDR3 comprising or consisting of the amino acid sequence ARDAGHGVDMDV (SEQ ID 3);

or

B) the antibody comprises at least one functionally active CDR variant of
a) the parent CDR1 consisting of the amino acid sequence of SEQ ID 1; or
b) the parent CDR2 consisting of the amino acid sequence of SEQ ID 2; or
c) the parent CDR3 consisting of the amino acid sequence of SEQ ID 3;

wherein the functionally active CDR variant comprises at least one point mutation in the parent CDR sequence, and comprises or consists of the amino acid sequence that has at least 60% sequence identity with the parent CDR sequence.

2. The antibody of definition 1, wherein the functionally active CDR variant comprises at least one of
a) 1, 2, or 3 point mutations in the parent CDR sequence; or
b) 1 or 2 point mutations in any of the four C-terminal or four N-terminal, or four centric amino acid positions of the parent CDR sequence.

3. The antibody of definition 1 or 2, wherein the functionally active CDR variant is any of
a) a CDR1 sequence selected from the group consisting of YPISSGMGWG (SEQ ID 4), and YSISSGMGWD (SEQ ID 5); or
b) a CDR2 sequence selected from the group consisting of SVDQRGSTYYNPSLKS (SEQ ID 6), RIDQRGSTYYNPSLKS (SEQ ID 7), RVDQRGSTYYNPSLKS (SEQ ID 8), SIDQRGSTYYNPSLEG (SEQ ID 9), and SIDQRGSTYYNPPLES (SEQ ID 10); or
c) a CDR3 sequence selected from the group consisting of ARDAGHGADMDV (SEQ ID 11), and ARDAGHAVDMDV (SEQ ID 12).

4. The antibody of any of definitions 1 to 3, which is selected from the group consisting of

a) an antibody comprising
a. the CDR1 sequence of SEQ ID 1; and
b. the CDR2 sequence of SEQ ID 6; and
c. the CDR3 sequence of SEQ ID 11;

b) an antibody comprising
a. the CDR1 sequence of SEQ ID 4; and
b. the CDR2 sequence of SEQ ID 7; and
c. the CDR3 sequence of SEQ ID 3;

c) an antibody comprising
a. the CDR1 sequence of SEQ ID 1; and
b. the CDR2 sequence of SEQ ID 8; and
c. the CDR3 sequence of SEQ ID 3;

d) an antibody comprising
a. the CDR1 sequence of SEQ ID 1; and
b. the CDR2 sequence of SEQ ID 2; and
c. the CDR3 sequence of SEQ ID 12;

e) an antibody comprising
a. the CDR1 sequence of SEQ ID 5; and
b. the CDR2 sequence of SEQ ID 9; and
c. the CDR3 sequence of SEQ ID 3;

f) an antibody comprising
a. the CDR1 sequence of SEQ ID 5; and
b. the CDR2 sequence of SEQ ID 10; and
c. the CDR3 sequence of SEQ ID 3;

5. The antibody of any of definitions 1 to 3, comprising a VH amino acid sequence selected from the group consisting of SEQ ID 20-31.

6. The antibody of any of definitions 1 to 3, comprising an antibody heavy chain (HC) amino acid sequence selected from the group consisting of SEQ ID 40-51, or any of the amino acid sequences of SEQ ID 40-51 with a deletion of the C-terminal amino acid.

7. The antibody of any of definitions 1 to 6, which further comprises at least three complementarity determining regions (CDR4 to CDR6) of the antibody light chain variable region (VL), preferably wherein

A) the antibody comprises
a) a CDR4 comprising or consisting of the amino acid sequence RASQGIRWLA(SEQ ID 32); and
b) a CDR5 comprising or consisting of the amino acid sequence AASSLQS (SEQ ID 33); and
c) a CDR6 comprising or consisting of the amino acid sequence QQGYVFPLT (SEQ ID 34);

or

B) the antibody comprises at least one functionally active CDR variant of
a) the parent CDR4 consisting of the amino acid sequence of SEQ ID 32; or
b) the parent CDR5 consisting of the amino acid sequence of SEQ ID 33; or
c) the parent CDR6 consisting of the amino acid sequence of SEQ ID 34;

wherein the functionally active CDR variant comprises at least one point mutation in the parent CDR sequence, and comprises or consists of the amino acid sequence that has at least 60% sequence identity with the parent CDR sequence.

8.1. The antibody of definition 7, comprising a VL amino acid sequence of SEQ ID 39 or an antibody light chain (LC) amino acid of SEQ ID 52.

8.2. The antibody of any of definitions 1 to 3, comprising a HC amino acid sequence selected from the group consisting of SEQ ID 40-51, optionally with a deletion of the C-terminal Lysine, and further comprising a LC amino acid of SEQ ID 52.

9. A cross-neutralizing antibody comprising at least one polyspecific binding site that binds to alpha-toxin (Hla) and at least one of the bi-component toxins of Staphylococcus aureus, which antibody is a functionally active variant antibody of a parent antibody that comprises a polyspecific binding site of the VH amino acid sequence of SEQ ID 20, and the VL amino acid sequence of SEQ ID 39, which functionally active variant antibody comprises at least one point mutation in any of the framework regions (FR) or constant domains, or complementarity determining regions (CDR1 to CDR6) in any of SEQ ID 20 or SEQ 39, and has an affinity to bind each of the toxins with a Kd of less than 10⁻⁸M, preferably less than 10⁻⁹M.

10. The antibody of definition 9, wherein the functionally active variant antibody comprises at least one of the functionally active CDR variants as defined in any of definitions 1 to 3.

11. The antibody of definition 9 or 10, wherein the functionally active variant antibody has a specificity to bind the same epitope as the parent antibody.

12. The antibody of any of definitions 1 to 11, wherein the at least one point mutation is any of an amino acid substitution, deletion and/or insertion of one or more amino acids.

13. The antibody of any of definitions 1 to 12, wherein the at least one point mutation is any of the amino acid substitutions
S51R or S51K in the CDR2; or
G103A, V104 Å or V104S in the CDR3.

14. The antibody of any of definitions 1 to 13, which has a cross-specificity to bind Hla and at least one of the F-components of the bi-component toxins, preferably at least two or three thereof.

15. The antibody of definition 14, wherein the F-components are selected from the group consisting of HlgB, LukF and LukD, or any F-component of the cognate and non-cognate pairs of F and S components of gamma-hemolysins, PVL toxins and PVL-like toxins, preferably HlgAB, HlgCB, LukSF, LukED, LukS-HlgB, LukSD, HlgA-LukD, HlgA-LukF, LukEF, LukE-HlgB, HlgC-LukD or HlgC-LukF.

16. The antibody of any of definitions 1 to 15, which inhibits the binding of one or more of the toxins to phosphocholine.

17. The antibody of any of definitions 1 to 16, which is a full-length monoclonal antibody, an antibody fragment thereof comprising at least one antibody domain incorporating the binding site, or a fusion protein comprising at least one antibody domain incorporating the binding site.

18. An expression cassette or a plasmid comprising a coding sequence to express a light chain and/or heavy chain of an antibody according to any of definitions 1 to 17.

19. A host cell comprising the expression cassette or the plasmid of definition 18.

20. A method of producing an antibody according to any of definitions 1 to 17, wherein a host cell according to definition 19 is cultivated or maintained under conditions to produce said antibody.

21. A method of producing functionally active antibody variants of a parent antibody which is any of the antibodies comprising a polyspecific binding site of the VH amino acid sequence of any of SEQ ID 20-31, and the VL amino acid sequence of SEQ ID 39, which method comprises engineering at least one point mutation in any of the framework regions (FR) or constant domains, or complementarity determining regions (CDR1 to CDR6) in any of SEQ ID 20-31 or SEQ 39 to obtain a variant antibody, and determining the functional activity of the variant antibody by any of
the affinity to bind each of Hla and at least one of the bi-component toxins of S. aureus with a Kd of less than 10⁻⁸M, preferably less than 10⁻⁹M, and/or
the binding of the variant antibody to Hla and/or the at least one of the bi-component toxins in competition with the parent antibody;

wherein upon determining the functional activity, the functionally active variants are selected for production by a recombinant production method.

22. The antibody according to any of definitions 1 to 16, for use in treating a subject at risk of or suffering from a S. aureus infection comprising administering to the subject an effective amount of the antibody to limit the infection in the subject, to ameliorate a disease condition resulting from said infection or to inhibit S. aureus disease pathogenesis, such as pneumonia, sepsis, bacteremia, wound infection, abscesses, surgical site infection, endothalmitis, furunculosis, carbunculosis, endocarditis, peritonitis, osteomyelitis or joint infection.

23. A pharmaceutical preparation comprising the antibody according to any of definitions 1 to 16, preferably comprising a parenteral or mucosal formulation, optionally containing a pharmaceutically acceptable carrier or excipient.

24. The antibody according to any of definitions 1 to 16, for diagnostic use to detect any S. aureus infections, including high toxin producing MRSA infections, such as necrotizing pneumonia, and toxin production in furunculosis and carbunculosis.

25. Diagnostic preparation of the antibody according to any of definitions 1 to 16, optionally containing the antibody with a label and/or a further diagnostic reagent with a label.

26. A crystal formed by a Hla monomer that diffracts x-ray radiation to produce a diffraction pattern representing the three-dimensional structure of the Hla rim domain in contact with the antibody of any of definitions 1 to 16, or a binding fragment thereof, preferably a Fab fragment, having the following cell constants: 285.05 Å, 150.94 Å, 115.25 Å, space group P2₁2₁2, optionally with a deviation of between 0.00 Å and 2.00 Å.

27. A crystal formed by a LukD monomer that diffracts x-ray radiation to produce a diffraction pattern representing the three-dimensional structure of the LukD rim domain in contact with the antibody of any of definitions 1 to 16, or a binding fragment thereof, preferably a Fab fragment, having the following cell constants: 112.0 Å, 112.0 Å, 409.3 Å, space group H32, optionally with a deviation of between 0.00 Å and 2.00 Å. 28. The isolated paratope of an antibody of any of definitions 1 to 16, or a binding molecule comprising said paratope.

29. An isolated conformational epitope recognized by the antibody of any of definitions 1 to 16, characterized by a three-dimensional structure of the rim domain of Hla, LukD, LukF or HlgB

30. The epitope of definition 29, characterized by a three-dimensional structure selected from the group consisting of
a) the three-dimensional Hla structure characterized by the structure coordinates of the contact amino acid residues 179-191, 194, 200, 269 and 271 of SEQ ID 54;
b) the three-dimensional LukF structure characterized by the structure coordinates of the contact amino acid residues 176-188, 191, 197 and 267 of SEQ ID 55, preferably with amino acid residues 176-179, 181-184, 186-188, 191, 197 and 267 of SE ID 58;
c) the three-dimensional LukD structure characterized by the structure coordinates of the contact amino acid residues 176-188, 191, 197 and 267 of SEQ ID 54, preferably with amino acid residues 176-179, 181-184, 186-188, 191, 197 and 267 of SEQ ID 62;
d) the three-dimensional HlgB structure characterized by the structure coordinates of the amino acid contact residues 177-189, 192, 198 and 268 of SEQ ID 56, preferably with amino acid residues 177-180, 182-185, 187-189, 192, 198 and 268 of SEQ ID 68,
e) the three-dimensional Hla rim domain structure of the crystal of definition 26;
f) the three-dimensional LukD rim domain structure of the crystal of definition 27; and
g) a three-dimensional structure which is a homolog of any of a) to f) wherein said homolog comprises a binding site that has a root mean square deviation from backbone atoms of contact amino acid residues of between 0.00 Å and 2.00 A.

31. The epitope of definitions 29 or 30, which is bound by a binding molecule.

32. A binding molecule which specifically binds to the epitope of definition 29 or 30, preferably selected from the group consisting of a protein, a peptide, a peptidomimetic, a nucleic acid, a carbohydrate, a lipid, an oligopeptide, an aptamer and a small molecule compound, preferably an antibody, an antibody fragment thereof comprising at least one antibody domain incorporating the binding site, or a fusion protein comprising at least one antibody domain incorporating the binding site.

33. The binding molecule of definitions 32, which is a polyspecific binder that binds to Hla and at least one of the bi-component toxins of S. aureus.

34. The binding molecule of definitions 32 or 33, which prevents toxin binding to phosphocholine and competes with the antibody of any of definition 1 to 16.

35. A screening method or assay for identifying a binder which specifically binds to the epitope of definition 29 or 30, comprising the steps of:
bringing a candidate compound into contact with the three-dimensional structure as defined in definition 29 or 30; and
assessing binding between the candidate compound and the three-dimensional structure; wherein binding between the candidate compound and the three-dimensional structure identifies the candidate compound as a polyspecific binder that binds to Hla and at least one of the bi-component toxins of S. aureus.

36. An immunogen comprising:
a) an epitope of definition 29 or 30;
b) optionally further epitopes not natively associated with said epitope of (a); and
c) a carrier, preferably a pharmaceutically acceptable carrier, preferably comprising buffer and/or adjuvant substances.

37. Immunogen according to definition 36 in a vaccine formulation, preferably for parenteral use.

38. Immunogen according to definition 36 or 37, for use in treating a subject by administering an effective amount of said immunogen to protect the subject from an S. aureus infection, to prevent a disease condition resulting from said infection or to inhibit S. aureus pneumonia pathogenesis.

39. Immunogen according to definition 38, for eliciting a protective immune response.

40. Isolated nucleic acid encoding an antibody according to any of definitions 1 to 16, or an epitope according to definition 29 or 30.

The foregoing description will be more fully understood with reference to the following examples. Such examples are, however, merely representative of methods of practicing one or more embodiments of the present invention and should not be read as limiting the scope of invention.

### EXAMPLES

### Example 1

### Generation of Hla—Bi-Component Toxin Cross-Reactive mAbs Binding with High Affinity to Individual Toxin Components

Methods:

Generation of Recombinant Toxins.

The genes for the S- and F-components were derived from the TCH1516 USA300 strain, codon optimized for E. coli expression, generated by gene synthesis (Genescript, USA), (see FIG. 7) cloned into pET44a and the proteins were produced in BL21, Rosetta or Tuner DE3 strains without signal peptide sequences (determined using the PrediSi program; Hiller, Nucleic Acids Res., 2004, 32: W375-W379)

LukS, LukF, LukE, LukD, HlgA, HlgC and HlgB were expressed in soluble form with an N-terminal NusA/His₆ tag which was removed proteolytically after the first purification step. Purification typically involved three chromatographic steps 1) IMAC (immobilized metal affinity column) 2) cation exchange or IMAC, and 3) size exclusion chromatography. The clarified cell extract was loaded onto a metal ion affinity column (IMAC 5 ml, GE Healthcare or Ni-IDA, 15 ml, Novagen) and the fusion protein was eluted with 500 mM imidazole. Following buffer exchange into cleavage buffer (20 mM Tris, pH 7.5, 200 mM NaCl, 2 mM CaCl₂) and digestion with enterokinase (New England Biolabs), the mature protein (containing two additional amino-acids at the N-terminus ‘SL’), was separated from the NusA/His₆ tag by metal ion affinity or cation exchange (SP-Sepharose FF, 5 ml, GE Healthcare or EMD SO³⁻, XK16 column, Merk) chromatography. The final purification step on the gel filtration column (Superdex 75 16/60, GE Healthcare) equilibrated in 50 mM sodium phosphate pH 7.5 plus 300 mM NaCl, yielded pure (>95%) proteins as judged by sodium dodecyl sulphate polyacrylamide gel electrophoresis (SDS-PAGE). The proteins were assayed for purity by SDS-PAGE, monomeric state by size exclusion chromatography, secondary structure by circular dichroism and for functionality in in vitro toxin potency assays.

For antibody selection, proteins were labeled with the amino reactive reagent Sulfo-NHS-LC biotin (Thermo-Scientific), according to the manufacturer's instructions, yielding biotin levels of 1-2.5 biotin/protein.

Selection of Monoclonal Antibodies.

Toxin-specific antibodies were isolated from a full length human IgG1 antibody library (W02009/036379A2; WO2010105256; WO2012009568) using an in vitro yeast selection system and associated methods. Toxin-binding mAbs were identified by incubating biotin labeled Hla or leukocidin monomers with antibody expressing yeast cells at different concentrations followed by magnetic bead selection and fluorescence-activated cell sorting (FACS) employing streptavidin secondary reagents in several successive selection rounds. Hla and bi-component toxin cross-reactive mAbs were selected by alternating use the different toxin baits. Antibodies were then produced by the selected yeast clones and purified by Protein A affinity chromatography.

Binding of mAbs to the different toxins was confirmed by interferometry measurements using a ForteBio Octet Red instrument (Pall Life Sciences). The biotinylated antigen or the antibody was immobilized on the sensor and the association and dissociation of the antibody Fab fragment or of the antigen, respectively (typically 200 nM), in solution, were measured. Fab KD affinities measured by MSD method using a Sector Immager 2400 instrument (Meso Scale Discovery). Typically 20 pM of biotinylated antigen was incubated with Fab at various concentrations, for 16 h at room temperature, and the unbound antigen captured on immobilized IgGSee also for example, Estep et al., “High throughput solution-based measurement of antibody-antigen affinity and epitope binning”, MAbs, Vol. 5(2), pp. 270-278 (2013).

Results:

A toxin cross-reactive mAb, AB-28 was discovered in successive rounds of selections first with Hla, followed by alternating interrogation with HlgB, LukF and LukD as antigens from a library of full length human IgG1 (approx. diversity ˜10⁻¹⁰) expressed on the surface of yeast. AB-28 displays high affinity towards Hla and HlgB (<10 pM), however, the LukF and LukD binding affinities are 1 and 2 log lower (>100 pM and >1 nM, respectively). Therefore, AB-28 was subjected to optimization using a human IgG1 library generated based on AB-28 CDR sequences. Affinity improved antibodies were obtained by successive selection with LukF, LukD, HlgB and Hla used at low concentrations. Antibodies with improved binding affinity towards LukF and/or LukD were identified by ForteBio or MSD based binding assays. Some antibodies displayed significant affinity improvement towards both LukF and LukD, while maintained the single digit picomolar affinity towards Hla and HlgB. Examples of such antibodies are shown in FIG. 1. The affinity improvement was achieved by single amino acid replacements in the CDR regions, as indicated in FIG. 1.

### Example 2

### Improved Binding Affinity of Hla—Bi-Component Toxin Cross-Reactive mAbs to LukF and LukD is Associated with Higher In Vitro Toxin Neutralizing Potency

Methods:

In Vitro Assays to Measure Toxin Mediated Cell Lysis.

Toxin potency towards target cells was assessed by measuring ATP levels of intoxicated cells (polymorphonuclear cells (PMNs), differentiated HL60 or A549 cells) or hemolysis activity on red blood cells. Briefly, Hla or an equimolar mixture of the F- and S components, were serially diluted in assay medium and used for intoxication of cells. Cell viability of PMNs, differentiated HL60 and A549 cells was then examined using a commercially available kit (Cell Titer-Glo® Luminescent Cell Viability Assay; Promega, USA) according to the manufacturer's instructions. Percent viability was calculated relative to mock-treated controls.

For Hla, two different in vitro assays were performed using either the human lung epithelial cell line A549 or rabbit red blood cells. A549 cells (HPACC #86012804) were trypsinized and plated on the preceding day at a density of 20,000 cells per well (96-well half area luminescence plates, Greiner, Austria) in F12K medium (Gibco, USA) supplemented with 10% FCS and Pen/Strep. Cells were intoxicated for 6 hours at 37° C.+5% CO₂ in F12K medium supplemented with 5% FCS and Pen/Strep.

For rabbit red blood cell assays, rabbit EDTA-whole blood was obtained from New Zealand White Rabbits (Preclinics GmbH, Germany). Blood was diluted 1:1 with PBS w/o Ca⁺⁺/Mg⁺⁺ (PAA Laboratories, Austria) and gradients were prepared by layering 15 ml diluted blood on 15 ml LSM 1077 (PAA Laboratories, Austria) in 50 ml polypropylene tubes. Following centrifugation at 680×g (RT, no brakes) platelets, plasma, PBMCs and Ficoll were removed by aspiration and discarded. The remaining RBC pellet was washed twice in 40 ml PBS w/o Ca⁺⁺/Mg⁺⁺ (centrifugation 680×g, RT, no brakes) and finally re-suspended in 20 ml PBS w/o Ca⁺⁺/Mg⁺⁺. Integrity and cell number of erythrocytes were determined in a standard hemocytometer. Hemolysis assay was performed with 5×10⁷ rabbit red blood cells diluted in PBS w/o Ca⁺⁺/Mg⁺⁺ for X hours at 37° C.+5% CO₂

To measure leukocidal activity of biocompenent toxins, either human PMNs cells or differentiated HL-60 cells were used for measuring cell lysis induced by recombinant toxins or S. aureus culture supernatants. Fresh human blood for PMN isolation was either obtained from the Red Cross (heparinized) or obtained by venipuncture from normal healthy volunteers in K-EDTA or Heparin vacutainer tubes (BD, USA). To aggregate erythrocytes 1 part HetaSep solution (Stem Cell Technologies, France) was added to 5 parts of blood, mixed and incubated at 37° C. until the plasma/erythrocyte interphase was at approximately 50% of the total volume. The leukocyte enriched plasma layer was then carefully layered on a 2-step Percoll gradient (73% and 63% Percoll Plus diluted in HBSS, GE Healthcare) and centrifuged at 680×g, RT, 30 min, no brakes. The first and second layer of the post-spin gradient (mainly serum and monocytes) were removed by aspiration. PMNs were harvested from the second opaque layer and washed twice in 50 ml HBSS (Gibco, USA)+10 mM Glucose. The number of viable cells was counted using trypan blue dye exclusion in a hemocytometer. For PMN ATP assays, cells were re-suspended in neutrophil medium, RPMI 1640 (PAA Laboratories, Austria) supplemented with 10% FCS, 2 mM L-Glutamine and 100 U/ml penicillin and 0.1 mg/ml streptomycin (PAA/GE Healthcare); The HL-60 (ATCC CCL-240™) human promyelocytic leukemia cell line was cultured in neutrophil medium and differentiated with 100 mM DMF (N,N-Dimethylformamide, Fisher BioReagents) or 4.3 mM dbcAMP (dibytiryl cyclic AMP; Sigma-Aldrich), as described previously (21,22). Differentiation was determined by disappearance of CD71 and appearance of CD11b staining using Brilliant Violet 421 conjugated anti-CD11b (clone ICRF44, BioLegend) and PE-conjugated anti-CD71 monoclonal antibodies (clone OKT9, eBioscience). PMN/HL60lysis assays were performed with 25,000 cells/well diluted in neutrophil medium in half area luminescence plates (Greiner, Austria) for 4 hours at 37° C.+5% CO₂.

Determining Toxin Neutralizing Activity of Antibodies.

For PMN/HL60 cell assays, antibodies were serially diluted in neutrophil medium and mixed with toxins at a fixed concentration as indicated in the figure legends. Viability assay was started after a 1 hour pre-incubation step to allow antibody-toxin binding. % inhibition of toxin activity was calculated using the following formula: % inhibition=[(viability toxin only−inhibited activity)/(viability toxin only)]×100. Neutralization activity in the graphs is represented as molar ratio of mAb:toxin at the IC50 mAb concentrationA human IgG1 control mAb expressed by yeast cells and generated against an irrelevant antigen (hen egg lysozyme) was included in all assays.

For A459 Hla-neutralization assays, cells were trypsinized and plated on the preceding day at a density of 20,000 cells per well (96-well half area luminescence plates, Greiner, Austria) in F12K medium (Gibco, USA) supplemented with 10% FCS and Pen/Strep). Antibodies were serially diluted in F12K medium supplemented with 5% FCS and Pen/Strep (=A549 cell assay medium) in a separate dilution plate and mixed with alpha hemolysin at a fixed concentration [3.03 nM]. After a 1 hour pre-incubation step at room temperature, seeding medium on adherent A549 cells was discarded and replaced by the mAb:toxin mixture. Cells were intoxicated for 6 hours at 37° C.+5% CO₂ and then subjected to ATP measurement (as described for PMNs/HL60).

For RBC hemolysis inhibition assays with monoclonal antibodies, antibodies were serially diluted in PBS and mixed with toxin at a fixed concentration as indicated in the figure legends. Hemolysis assay was started after a 1 hour pre-incubation step to allow antibody-toxin binding. % inhibition of toxin activity was calculated using the following formula: % inhibition=[(hemolysis toxin only−inhibited activity)/(hemolysis toxin only)]×100. Neutralization activity in the graphs is represented as molar ratio of mAb:toxin at the IC50 mAb concentration.

Results:

Toxin neutralizing potency of antibodies was measured by intoxication of human PMNs with recombinant LukSF, LukED and HlgCB, or that of human red blood cells with HlgAB or that of human lung epithelial cells (A549 cell line) with Hla. Binding affinity towards the different toxins predicted very well the toxin neutralization potency of mAbs, as shown in FIG. 2. The best Hla-LukF-LukD-HlgB quadruple cross-reactive antibodies with K_{D} values for LukD <800 pM and for LukF <55 pM, exemplified by AB-28-6, AB-28-7, AB-28-8 and AB-28-9, were highly efficient in neutralizing all targeted toxins.

The potency of F-component specific mAbs was not restricted to the four cognate toxin pairs but was equally evident against leukocidins formed by non-cognate pairing, such as HlgA-LukD.

The benefit of improved toxin cross-reactivity is highlighted by the results of intoxication assays performed with a mixture of recombinant leukocidins active against a certain cell type, added at concentrations sufficient to cause 100% cell lysis by the individual toxins. Antibodies that lack appreciable activity even against only one of the toxins failed to provide protection against cell lysis. mAbs with the highest overall affinity against F-components and Hla—exemplified by AB-28-6, AB-28-7, AB-28-8 and AB-28-9—were found to have superior potency in these combined toxin assays, shown in FIG. 3.

### Example 3

### Improved Binding Affinity of Hla—Bi-Component Toxin Cross-Reactive mAbs to LukF and LukD is Associated with Higher In Vivo Protection

Methods:

Passive Protection of Mice with Monoclonal Antibodies.

The protective effects of anti-S. aureus toxin antibodies were evaluated in several murine models. Passive immunization with mAbs was performed intraperitoneally 24 h prior to the lethal challenge with recombinant toxins. Groups of 5 mice (BALB/c) received 5 or 10 mg/kg doses (100 or 200 μg/mouse, respectively) of the individual mAbs diluted in PBS. Control groups received either PBS alone or the same dose of isotype matched non-specific mAb. Challenge was performed intravenously with HlgA-HlgB or HlgA-LukD toxin pairs at 0.2 and 1 μg (each component) per mouse doses, respectively.

Results:

Cross-reactive Hla mAbs with <20 pM affinity to HlgB were highly effective to prevent lethality due to exposure to recombinant HlgAB, as shown in FIG. 4 with mAbs AB-28-3 (K_{D}=18 pM) and AB-28-9 (K_{D}=5 pM).

The cross-reactive mAbs displayed the broadest range of affinities towards LukD from 85 pM to 2.2 nM. The tested offsprings generated from the AB-28 parent mAb by changing certain amino acids in the CDR regions are significantly protective with strong correlation between affinity and in vivo efficacy exemplified by AB-28-9, AB-28-7 and AB-28-8, having 400, 290 and 780 pM K_{D} values and 75, 100 and 35% survival rates, respectively, as shown in FIG. 5.

### Example 4

### Protection Mediated by Hla—Bi-Component Toxin Cross-Reactive mAbs Against Intranasal Bacterial Challenge by S. aureus TCH1516

Methods:

Passive protection of mice with monoclonal antibodies. The protective effects of anti-S. aureus toxin antibodies in a murine pneumonia model was assessed. Passive immunization with mAbs was performed intraperitoneally 24 h prior to lethal intranasal challenge with live bacteria. Groups of 5 mice (BALB/c) received 5 mg/kg doses (100 μg; 0.2 mg/ml) of the individual mAbs diluted in PBS. Control groups received PBS alone. 40 μl of bacterial suspension containing 6×10⁸ cfu was applied to the nares after anesthetizing the mice with 10% Ketamin-2% Rompun.

Results:

Cross-reactive Hla mAbs were highly effective in preventing lethality induced by S. aureus TCH1516 in a pneumonia model. All antibodies exhibited a high level of protection when compared to the control group which received vehicle only (20% survival).

### Example 5

### Epitope Mapping/Binding of Antibodies Using the Crystal Structure of Hla:AB-28 and LukD:AB-28 Complexes and Phosphocholine Binding Assays

The epitope residues of the AB-28 antibody in the Hla and LukD molecules were identified from the crystal structures of Hla and LukD, respectively, in complex with the Fab fragment of AB-28. The epitopes are defined as the toxin residues at the Fab-toxin interface which are in contact with the Fab residues, i.e. the distance between any of their non-hydrogen atoms is smaller or equal to 4.0 Å, as measured in Pymol (PyMOL Molecular Graphics System, Version 1.5.0.4 Schrödinger, LLC).

The Hla epitope depicted in FIG. 9A, is found in the rim domain of Hla (sphere representation), and is binding to residues from both the light chain (black cartoon) and heavy chain (grey, cartoon) of the variable domain of the AB-28 Fab. The Hla contact residues (FIG. 8B, spheres) determined from the Hla crystal structure are aminoacids: 179-191, 194, 200, 269 and 271. Among these, amino acids 179-182, 184-186, 191, 200, 269 and 271 (FIG. 9B, black spheres) are fully conserved between Hla, LukF, LukD and HlgB, while amino acids 183, 190 and 194 are conserved between LukF, LukD and HlgB, and amino acid 189 conserved between Hla, LukD and HlgB, based on sequence alignments and available structural data. The aminoacid 179 (Trp in Hla) corresponds to other aromatic residues in LukD and LukF (Tyr) and HlgB (Phe). The corresponding amino acids in LukF and LukD are 176-188, 191, 197, 265 and 267, while those in HlgB are 177-189, 192, 198, 266 and 268.

The contact amino acids determined from the LukD:AB-28 crystal structure (FIG. 10A, same representation as in FIG. 8A) in LukD, are: 176-179, 181-184, 186-188, 191, 197 and 267 (FIG. 10A, contact residues as spheres, fully conserved residues in black), all except 184 (different in Hla and HlgB) and 187 and 191 (different in Hla) being fully conserved among Hla, LukF, LukD and HlgB.

Phosphocholine binding to the rim domain, in a pocket conserved between Hla, LukF, LukD and HlgB, was observed crystallographically for Hla (Science 1996, 274, 1859), HlgB (Nat. Struct. Biol. 1999, 6, 134) and LukF (Structure, 1999, 7, 277-287). The amino acids involved in phosphocholine binding in LukF are Asn-173, Trp-176, Tyr-179, Glu-191 and Arg197, the latter four being fully conserved between Hla, LukF, LukD and HlgB, and also contact residues between LukD (and Hla) and AB-28 in the crystal structures. Binding of phosphocholine to the toxins was assessed in ForteBio-based measurements by immobilizing biotinylated toxins on streptavidin sensors and measuring the binding of a phosphocholine-BSA adduct (PC4-BSA, Biosearch Technologies) in solution (PBS+1% BSA). There is measurable binding of PC4-BSA to LukF, LukD and HlgB, but not to the negative control S component HlgA (FIG. 11), as judged from the response values measure with the ForteBio analysis software version 7. The binding of the AB-28 IgG (10 μg/ml) to the biotinylated toxins prevents subsequent binding of phosphocholine (FIG. 11), which indicates that AB-28 outcompetes phosphocholine binding to the toxins.

### Example 6

### In Silico Analysis of Variant Amino Acids Using the Crystal Structure of Hla:AB-28 and LukD:AB-28 Complexes

The crystal structures of the AB-28 Fab fragment in complex with Hla and LukD were used to calculate binding energies for all contact positions as well as for in silico mutagenesis of the Fab contact residues to predict variants with improved binding towards one or both toxins.

The structures were prepared using YASARA (Krieger E, Vriend G. YASARA View-molecular graphics for all devices-from smartphones to workstations. Bioinformatics. 2014 Oct. 15; 30(20):2981-2), the ions and water molecules were stripped, the hydrogens were added and optimized, using steepest descent minimization. The contributions of each residue to the overall binding energy were calculated using Rosetta score 12 function (Kaufmann K W, Lemmon G H, Deluca S L, Sheehan J H, Meiler J. Practically useful: what the Rosetta protein modeling suite can do for you. Biochemistry. 2010 Apr. 13; 49(14):2987-98) without further optimization.

The energetics for each contact residue are given in Table 1 below. There are several positions in contact but with low contribution to the overall binding energy, particularly in VL CDR5 which could present opportunities for increasing affinity upon mutagenesis. Several methods were tested for the in silico mutagenesis protocol and the method that exhibited best qualitative agreement with the known binding characteristics of AB-28 variants was chosen. Selected contact positions were mutated to all amino acids except Cys and Pro and the binding energy changes for each mutation are given in Table 2.1 and 2.2 below.

TABLE 1
Binding energies (ΔG values) of AB-28 contact residues to LukD and Hla
Light chain

| | VL CDR4 | | | VL FR2 | VL CDR5 | | | VL FR3 | VL CDR6 | | |
|---|---|---|---|---|---|---|---|---|---|---|---|
| Target | S7 | R8 | W9 | Y15 | A1 | S3 | S4 | S4 | Y4 | V5 | F6 |
| LukD | −0.03 | −0.19 | −3.67 | −0.13 | −0.10 | — | −0.02 | 0.00 | −1.26 | −0.38 | −1.62 |
| Hla | — | −1.01 | −4.34 | −0.09 | −0.16 | 0.02 | 0.01 | — | −2.04 | −0.50 | −1.28 |

Heavy chain

| | VH CDR1 | | VH CDR2 | | | | | VH CDR3 | | | | | |
|---|---|---|---|---|---|---|---|---|---|---|---|---|---|
| Target | S5 | M7 | D3 | Q4 | R5 | S7 | Y9 | D3 | A4 | G5 | H6 | G7 | V8 |
| LukD | 0.00 | −0.93 | 0.22 | −2.08 | −2.10 | 0.03 | 0.03 | 0.02 | — | −0.22 | −1.50 | −0.20 | −0.38 |
| Hla | 0.01 | −0.98 | −0.49 | −2.71 | −1.35 | −0.33 | −0.59 | — | 0.02 | −1.73 | −3.24 | −1.72 | −0.38 |

TABLE 2.1
Change in binding energies (ΔΔG) upon mutation of
AB-28 contact residues to LukD and Hla - Light Chain
Light chain

| Antigen | Region | Number | AA | A | D | E | F | G | H | I | K | L |
|---|---|---|---|---|---|---|---|---|---|---|---|---|
| Hla | VL CDR4 | 7 | S | 0.02 | 0.02 | −0.14 | −0.16 | −0.24 | −0.04 | −0.16 | −0.47 | −0.50 |
| | VL CDR4 | 8 | R | 1.76 | 1.95 | 1.93 | 1.76 | 1.86 | 1.60 | 1.72 | 2.32 | 1.76 |
| | VL CDR4 | 9 | W | 2.22 | 1.76 | 2.19 | 7.18 | 2.52 | 3.81 | 1.43 | 4.36 | 3.48 |
| LukD | VL CDR4 | 7 | S | 0.16 | 0.76 | 0.11 | 0.07 | 0.16 | 0.94 | 0.84 | 0.05 | 0.11 |
| | VL CDR4 | 8 | R | 0.96 | 1.04 | 0.99 | 0.97 | 1.03 | 0.86 | 0.97 | 0.83 | 0.96 |
| | VL CDR4 | 9 | W | 4.25 | 3.47 | 3.60 | 2.70 | 4.45 | 1.73 | 2.84 | 2.95 | 2.82 |
| Hla | VL CDR5 | 1 | A | 0.00 | 0.58 | 2.54 | 1.37 | 0.07 | 1.24 | 0.06 | 2.10 | 5.10 |
| | VL CDR5 | 3 | S | 0.03 | 0.09 | 0.04 | 0.08 | −0.09 | 0.00 | 0.03 | 0.04 | 0.03 |
| | VL CDR5 | 4 | S | −0.02 | 0.08 | −0.18 | 2.19 | −0.02 | −0.26 | −0.06 | −0.50 | −0.02 |
| LukD | VL CDR5 | 1 | A | 0.00 | 1.39 | 3.26 | 0.96 | −0.61 | 1.49 | 2.86 | 6.54 | 3.19 |
| | VL CDR5 | 3 | S | −0.04 | −0.04 | 1.25 | 0.79 | −0.04 | −0.45 | −0.15 | −0.04 | −0.04 |
| | VL CDR5 | 4 | S | 0.54 | 0.45 | 0.35 | 0.44 | 0.57 | 0.26 | 0.33 | −0.23 | 2.33 |
| Hla | VL CDR6 | 3 | G | −0.09 | −0.06 | −0.06 | −0.06 | 0.00 | −0.06 | 0.10 | −0.07 | −0.07 |
| | VL CDR6 | 4 | Y | 0.74 | 0.48 | 0.56 | −0.05 | 0.76 | 0.06 | 0.51 | 0.70 | 0.46 |
| | VL CDR6 | 5 | V | 0.09 | −0.03 | −0.29 | 0.97 | −0.19 | −0.09 | −0.19 | −0.19 | −0.18 |
| | VL CDR6 | 6 | F | 0.98 | 0.77 | 1.08 | 0.00 | 1.08 | 0.18 | 1.12 | 1.79 | 0.18 |
| LukD | VL CDR6 | 3 | G | −0.05 | −0.04 | −0.04 | −0.20 | 0.00 | −0.20 | 0.23 | −0.04 | −0.20 |
| | VL CDR6 | 4 | Y | 0.38 | 0.21 | 0.24 | −0.01 | 0.43 | −0.20 | −0.19 | −0.17 | 0.52 |
| | VL CDR6 | 5 | V | 0.01 | 0.02 | −0.37 | −0.66 | 0.03 | −0.24 | −0.27 | −0.12 | −0.24 |
| | VL CDR6 | 6 | F | 1.09 | 1.03 | 1.24 | 0.00 | 1.23 | 0.92 | 0.88 | 0.44 | 0.57 |

| Antigen | Region | M | N | Q | R | S | T | V | W | Y |
|---|---|---|---|---|---|---|---|---|---|---|
| Hla | VL CDR4 | −0.71 | 0.02 | −0.17 | −0.61 | 0.00 | 0.06 | 0.16 | −1.85 | −0.18 |
| | VL CDR4 | 1.58 | 1.69 | 1.74 | 0.00 | 1.64 | 1.72 | 1.76 | 1.76 | 1.73 |
| | VL CDR4 | 2.53 | 2.29 | 2.70 | 2.92 | 2.17 | 2.06 | 1.72 | 0.00 | 7.97 |
| LukD | VL CDR4 | −0.10 | 0.12 | 0.07 | −0.86 | 0.00 | 0.83 | 0.84 | −0.33 | −0.24 |
| | VL CDR4 | 0.91 | 0.91 | 0.87 | 0.00 | 0.94 | 0.92 | 0.96 | 0.90 | 0.97 |
| | VL CDR4 | 2.52 | 4.81 | 3.64 | 2.73 | 4.23 | 3.73 | 3.11 | 0.00 | 2.88 |
| Hla | VL CDR5 | 3.71 | 1.24 | 1.82 | 4.43 | −0.02 | 1.77 | 1.94 | 1.46 | 0.50 |
| | VL CDR5 | 0.04 | −0.02 | 0.08 | 0.04 | 0.00 | 0.01 | 0.03 | 0.05 | 0.15 |
| | VL CDR5 | −0.13 | −0.69 | −1.34 | 0.54 | 0.00 | 0.04 | −0.08 | 1.19 | 3.96 |
| LukD | VL CDR5 | 3.34 | 1.73 | 3.72 | 5.59 | 1.48 | 1.71 | 1.91 | 13.46 | 4.97 |
| | VL CDR5 | 0.51 | −0.07 | 0.00 | −0.04 | 0.00 | 0.16 | −0.12 | −0.04 | 0.98 |
| | VL CDR5 | 0.41 | 0.36 | 0.20 | −0.55 | 0.00 | −0.19 | 0.15 | 0.05 | 0.17 |
| Hla | VL CDR6 | −0.09 | −0.07 | −0.06 | −0.18 | −0.06 | −0.05 | −0.14 | −0.06 | −0.06 |
| | VL CDR6 | 0.43 | 0.64 | 0.54 | 0.43 | 0.66 | 0.56 | 0.52 | 0.17 | 0.00 |
| | VL CDR6 | −0.10 | 0.11 | −0.35 | −0.17 | 0.15 | 0.23 | 0.00 | 0.35 | 1.04 |
| | VL CDR6 | 1.54 | 0.81 | 1.17 | 1.39 | 0.97 | 0.71 | 0.58 | −0.29 | 0.03 |
| LukD | VL CDR6 | 2.04 | −0.04 | −0.04 | −0.04 | −0.04 | −0.05 | −0.20 | −0.20 | −0.20 |
| | VL CDR6 | 0.02 | 0.32 | 0.24 | 0.07 | 0.42 | 0.25 | 0.20 | 0.14 | 0.00 |
| | VL CDR6 | −0.42 | 0.03 | −0.21 | −0.13 | −0.01 | −0.01 | 0.00 | −0.45 | −0.34 |
| | VL CDR6 | 1.11 | 0.97 | 0.75 | 0.98 | 1.03 | 0.87 | 0.87 | −0.09 | 0.52 |

TABLE 2.2
Change in binding energies (ΔΔG) upon mutation of
AB-28 contact residues to LukD and Hla - Heavy Chain
Heavy chain

| Antigen | Region | Number | AA | A | D | E | F | G | H | I | K | L |
|---|---|---|---|---|---|---|---|---|---|---|---|---|
| Hla | VH CDR1 | 5 | S | 0.08 | 0.06 | 0.07 | 0.08 | 0.08 | 0.07 | 0.07 | 0.07 | 0.07 |
| | VH CDR1 | 7 | M | 1.27 | 1.22 | 0.61 | 0.40 | 1.26 | −1.55 | 0.55 | −0.27 | 0.56 |
| LukD | VH CDR1 | 5 | S | 0.17 | 0.06 | 0.15 | 0.09 | 0.17 | −0.62 | 0.05 | 0.17 | 0.13 |
| | VH CDR1 | 7 | M | 1.42 | 1.14 | 0.88 | 1.25 | 1.39 | 0.17 | 0.27 | −0.90 | 0.53 |
| Hla | VH CDR2 | 3 | D | 2.28 | 0.00 | 2.29 | 2.61 | 2.33 | 1.88 | 3.23 | 2.84 | 3.78 |
| | VH CDR2 | 4 | Q | 3.50 | 3.40 | 1.29 | 1.65 | 3.65 | 2.07 | 2.15 | 3.42 | 2.79 |
| | VH CDR2 | 5 | R | 2.28 | 2.88 | 1.83 | 1.66 | 5.16 | −0.44 | 2.60 | 3.19 | 0.42 |
| | VH CDR2 | 7 | S | 0.40 | −1.11 | −0.31 | 0.31 | 0.66 | −1.25 | 2.31 | 0.07 | 2.88 |
| | VH CDR2 | 9 | Y | −0.11 | −0.11 | −0.16 | 0.47 | 0.30 | 0.57 | 0.33 | −0.20 | 0.48 |
| LukD | VH CDR2 | 3 | D | 1.07 | 0.00 | 1.10 | 2.94 | 1.11 | −0.09 | 0.94 | 0.52 | 1.27 |
| | VH CDR2 | 4 | Q | 2.13 | 2.20 | 1.29 | 2.32 | 2.43 | 0.92 | 1.49 | 2.98 | 2.52 |
| | VH CDR2 | 5 | R | 3.16 | 2.33 | 2.10 | 2.13 | 4.56 | 0.92 | 3.26 | 2.26 | 1.83 |
| | VH CDR2 | 7 | S | 0.12 | 0.03 | −0.30 | −0.12 | 0.28 | −0.39 | 0.08 | −0.65 | −0.15 |
| | VH CDR2 | 9 | Y | 0.66 | 0.64 | 0.56 | 0.07 | 0.66 | 0.20 | 0.65 | 0.38 | 0.25 |
| Hla | VH CDR3 | 5 | G | 0.14 | −0.96 | 2.05 | 0.23 | 0.00 | −2.64 | −1.25 | 0.91 | 1.56 |
| | VH CDR3 | 6 | H | 1.94 | 2.28 | 2.18 | 1.51 | 2.52 | 0.00 | 2.40 | 1.52 | 2.15 |
| | VH CDR3 | 7 | G | −0.37 | −0.44 | −0.50 | 4.80 | 0.00 | −0.02 | −0.40 | 4.49 | 1.71 |
| | VH CDR3 | 8 | V | 0.01 | −0.08 | −0.28 | 1.95 | 0.12 | −0.22 | −0.31 | −0.34 | 0.29 |
| LukD | VH CDR3 | 5 | G | −0.11 | 0.03 | 0.14 | −0.61 | 0.00 | −1.49 | −0.17 | 0.40 | −0.11 |
| | VH CDR3 | 6 | H | 0.66 | 0.95 | −1.40 | 2.86 | 1.17 | 0.00 | 0.72 | −0.33 | 0.30 |
| | VH CDR3 | 7 | G | −0.30 | −0.29 | −0.32 | −0.30 | 0.00 | −0.30 | −0.14 | −0.18 | −0.30 |
| | VH CDR3 | 8 | V | −0.05 | −0.06 | 0.15 | −0.07 | 0.03 | 2.99 | −0.30 | −0.31 | −0.22 |

| Antigen | Region | M | N | Q | R | S | T | V | W | Y |
|---|---|---|---|---|---|---|---|---|---|---|
| Hla | VH CDR1 | 0.08 | 0.06 | 0.08 | 0.08 | 0.00 | −0.01 | 0.07 | 0.07 | 0.07 |
| | VH CDR1 | 0.00 | 1.07 | −0.20 | −1.15 | 1.20 | 1.14 | 1.20 | −2.01 | 0.95 |
| LukD | VH CDR1 | 0.16 | 0.11 | 0.16 | −1.55 | 0.00 | −0.03 | 0.04 | −1.27 | 0.08 |
| | VH CDR1 | 0.00 | 0.97 | 0.36 | 0.00 | 1.41 | 1.50 | 1.11 | 0.48 | 1.18 |
| Hla | VH CDR2 | 2.20 | 2.41 | 1.98 | 2.89 | 2.11 | 2.28 | 2.29 | 4.39 | 3.37 |
| | VH CDR2 | 2.63 | 3.55 | 0.00 | 2.12 | 3.58 | 3.09 | 2.75 | 5.98 | 1.87 |
| | VH CDR2 | 0.36 | 2.75 | 1.85 | 0.00 | 2.52 | 1.65 | 1.12 | 0.56 | 1.51 |
| | VH CDR2 | 0.27 | −0.52 | −0.55 | 0.24 | 0.00 | 0.25 | 2.34 | 0.47 | 0.34 |
| | VH CDR2 | −0.44 | −0.20 | −0.33 | −0.59 | −0.14 | 0.13 | 0.40 | 0.75 | 0.00 |
| LukD | VH CDR2 | 1.20 | 0.49 | 0.07 | −0.78 | 0.73 | 1.06 | 0.99 | 0.61 | 2.88 |
| | VH CDR2 | 2.23 | 1.97 | 0.00 | 2.27 | 1.84 | 1.58 | 2.10 | 1.54 | 1.88 |
| | VH CDR2 | 2.09 | 2.57 | 2.27 | 0.00 | 3.46 | 2.21 | 2.18 | 2.81 | 2.97 |
| | VH CDR2 | −0.18 | 0.04 | −0.73 | 0.03 | 0.00 | 0.04 | 0.07 | −0.03 | −0.13 |
| | VH CDR2 | 0.64 | 0.56 | 0.40 | 0.41 | 0.54 | 0.58 | 0.65 | 0.74 | 0.00 |
| Hla | VH CDR3 | −1.52 | −1.31 | 0.81 | 1.13 | −0.13 | −0.78 | −0.93 | 1.06 | 0.18 |
| | VH CDR3 | 2.07 | 2.84 | 1.56 | 1.62 | 0.35 | 2.04 | 2.83 | 3.78 | 4.65 |
| | VH CDR3 | −0.30 | −0.29 | −0.27 | 6.61 | −0.28 | −0.36 | −0.26 | 4.67 | 4.70 |
| | VH CDR3 | −0.66 | 0.05 | −0.18 | −0.65 | −0.04 | −0.21 | 0.00 | 4.21 | 2.44 |
| LukD | VH CDR3 | −0.42 | −0.17 | 0.41 | −0.94 | −0.28 | 0.17 | 0.23 | −0.02 | −1.53 |
| | VH CDR3 | 0.21 | 1.05 | −1.05 | 0.29 | 0.19 | 0.07 | 0.36 | 0.36 | 2.55 |
| | VH CDR3 | −0.30 | −0.38 | −0.38 | −0.32 | −0.32 | −0.29 | −0.14 | −0.57 | −0.30 |
| | VH CDR3 | −0.78 | 0.64 | −0.46 | −2.70 | −0.06 | −0.06 | 0.00 | 3.28 | 0.82 |

The in silico mutagenesis indicated that changing several AB-28 contact residues could lead to improved binding to both Hla and LukD, i.e. S7R in VL CDR4 and S7Q in VH CDR2, V8M, V8R in VH CDR3. A number of other mutations: S7W, S7M, S7L in VL CDR4, S4Q, S4N, S4K in VL CDR5 and M7H, M7R in VH CDR1, S7D, S7H, S7N, Y9R in VH CDR2 predict better binding to Hla, without affecting binding to LukD. Likewise, the A1G substitution in VL CDR5 and S5H, S5R, S5W, M7K substitutions in VH CDR1, S7K substitution in VH CDR2 may lead to better binding to LukD, without affecting binding to Hla. On the other hand the S4R substitution in VL CDR5 and H6E, H6Q substitutions in VH CDR3 are predicted to improve binding to LukD but decrease binding to Hla. There are also a relatively high number of mutations that are not affecting binding to either LukD or Hla (ΔΔG values <0.5), so these variants are expected to show similar binding profiles as AB-28.

1. A cross-neutralizing antibody comprising at least one polyspecific binding site that binds to alpha-toxin (Hla) and at least one of the bi-component toxins of Staphylococcus aureus, which antibody comprises at least three complementarity determining regions (CDR1 to CDR3) of the antibody heavy chain variable region (VH), wherein
A) the antibody comprises
a) a CDR1 comprising or consisting of the amino acid sequence YSISSGMGWG (SEQ ID 1); and
b) a CDR2 comprising or consisting of the amino acid sequence SIDQRGSTYYNPSLKS (SEQ ID 2); and
c) a CDR3 comprising or consisting of the amino acid sequence ARDAGHGVDMDV (SEQ ID 3);
or
B) the antibody comprises at least one functionally active CDR variant of
a) the parent CDR1 consisting of the amino acid sequence of SEQ ID 1; or
b) the parent CDR2 consisting of the amino acid sequence of SEQ ID 2; or
c) the parent CDR3 consisting of the amino acid sequence of SEQ ID 3;
wherein the functionally active CDR variant comprises at least one point mutation in the parent CDR sequence, and comprises or consists of the amino acid sequence that has at least 60% sequence identity with the parent CDR sequence,
wherein the antibody is not antibody #AB-24 produced by a host cell comprising
i) an antibody light chain designated #AB-24-LC which coding sequence is comprised in the host cell deposited under DSM 26748, and
ii) an antibody heavy chain designated #AB-24-HC which coding sequence is comprised in the host cell deposited under DSM 26747.

2. The antibody of claim 1, wherein
a) in VH CDR1 at position 5, the amino acid residue is selected from the group consisting of S, A, D, E, F, G, H, I, K, L, M, N, Q, R, T V, W and Y;
b) in VH CDR1 at position 7, the amino acid residue is selected from the group consisting of M, H, K, Q, R and W;
c) in VH CDR2 at position 3, the amino acid residue is selected from the group consisting of D and R;
d) in VH CDR2 at position 7, the amino acid residue is selected from the group consisting of S, A, D, E, F, H, K, M, N, Q, R, T, W and Y;
e) in VH CDR2 at position 9, the amino acid residue is selected from the group consisting of Y, F, K, L, Q and R;
f) in VH CDR3 at position 5, the amino acid residue is selected from the group consisting of G, A, D, F, H, I, M, N, R, S, T, V and Y;
g) in VH CDR3 at position 6, the amino acid residue is selected from the group consisting of H, E, Q and S;
h) in VH CDR3 at position 7, the amino acid residue is selected from the group consisting of G, A, D, E, H, I, M, N, Q, S, T, V and W; and/or
i) in VH CDR3 at position 8, the amino acid residue is selected from the group consisting of V, A, D, E, G, I, K, L, M, Q, R, S and T.

3. The antibody of claim 1, wherein the functionally active CDR variant comprises at least one of
a) 1, 2, or 3 point mutations in the parent CDR sequence; or
b) 1 or 2 point mutations in any of the four C-terminal or four N-terminal, or four centric amino acid positions of the parent CDR sequence.

4. The antibody of claim 1, wherein the functionally active CDR variant is any of
a) a CDR1 sequence selected from the group consisting of YPISSGMGWG (SEQ ID 4), and YSISSGMGWD (SEQ ID 5); or
b) a CDR2 sequence selected from the group consisting of SVDQRGSTYYNPSLKS (SEQ ID 6), RIDQRGSTYYNPSLKS (SEQ ID 7), RVDQRGSTYYNPSLKS (SEQ ID 8), SIDQRGSTYYNPSLEG (SEQ ID 9), and SIDQRGSTYYNPPLES (SEQ ID 10); or
c) a CDR3 sequence selected from the group consisting of ARDAGHGADMDV (SEQ ID 11), and ARDAGHAVDMDV (SEQ ID 12).

5. The antibody of claim 1, which is selected from the group consisting of
a) an antibody comprising
a. the CDR1 sequence of SEQ ID 1; and
b. the CDR2 sequence of SEQ ID 6; and
c. the CDR3 sequence of SEQ ID 11;
b) an antibody comprising
a. the CDR1 sequence of SEQ ID 4; and
b. the CDR2 sequence of SEQ ID 7; and
c. the CDR3 sequence of SEQ ID 3;
c) an antibody comprising
a. the CDR1 sequence of SEQ ID 1; and
b. the CDR2 sequence of SEQ ID 8; and
c. the CDR3 sequence of SEQ ID 3;
d) an antibody comprising
a. the CDR1 sequence of SEQ ID 1; and
b. the CDR2 sequence of SEQ ID 2; and
c. the CDR3 sequence of SEQ ID 12;
e) an antibody comprising
a. the CDR1 sequence of SEQ ID 5; and
b. the CDR2 sequence of SEQ ID 9; and
c. the CDR3 sequence of SEQ ID 3;
f) an antibody comprising
a. the CDR1 sequence of SEQ ID 5; and
b. the CDR2 sequence of SEQ ID 10; and
c. the CDR3 sequence of SEQ ID 3;

6. The antibody of claim 1, comprising a VH amino acid sequence selected from the group consisting of SEQ ID 20-31, and comprising an antibody heavy chain (HC) amino acid sequence selected from the group consisting of SEQ ID 40-51, or any of the amino acid sequences of SEQ ID 40-51 with a deletion of the C-terminal amino acid.

7. The antibody of claim 1, which further comprises at least three complementarity determining regions (CDR4 to CDR6) of the antibody light chain variable region (VL), wherein
A) the antibody comprises
a) a CDR4 comprising or consisting of the amino acid sequence RASQGISRWLA(SEQ ID 32); and
b) a CDR5 comprising or consisting of the amino acid sequence AASSLQS(SEQ ID 33); and
c) a CDR6 comprising or consisting of the amino acid sequence QQGYVFPLT(SEQ ID 34);
or
B) the antibody comprises at least one functionally active CDR variant of
a) the parent CDR4 consisting of the amino acid sequence of SEQ ID 32; or
b) the parent CDR5 consisting of the amino acid sequence of SEQ ID 33; or
c) the parent CDR6 consisting of the amino acid sequence of SEQ ID 34;
wherein the functionally active CDR variant comprises at least one point mutation in the parent CDR sequence, and comprises or consists of the amino acid sequence that has at least 60% sequence identity with the parent CDR sequence.

8. The antibody according to claim 7, wherein
a) in VL CDR4 at position 7, the amino acid residue is selected from the group consisting of S, A, E, F, G, K, L, M, N, Q, R, W and Y;
b) in VL CDR5 at position 1, the amino acid residue is selected from the group consisting of A and G;
c) in VL CDR5 at position 3, the amino acid residue is selected from the group consisting of S, A, D, G, H, I, K, L, N, Q, R, T, V and W;
d) in VL CDR5 at position 4, the amino acid residue is selected from the group consisting of S, D, E, H, I, K, M, N, Q, R, T and V;
e) in VL CDR6 at position 3, the amino acid residue is selected from the group consisting of G, A, D, E, F, H, I, K, L, N, Q, R, S, T, V, W and Y;
f) in VL CDR6 at position 4, the amino acid residue is selected from the group consisting of Y, D, F, H, M, R and W;
g) in VL CDR6 at position 5, the amino acid residue is selected from the group consisting of V, A, D, E, F, G, H, I, K, L, M, N, Q, R, S, T, and W; and/or
h) in VL CDR6 at position 6, the amino acid residue is selected from the group consisting of F and W.

9. The antibody of claim 7, comprising a VL amino acid sequence of SEQ ID 39 or an antibody light chain (LC) amino acid of SEQ ID 52.

10. A cross-neutralizing antibody comprising at least one polyspecific binding site that binds to alpha-toxin (Hla) and at least one of the bi-component toxins of Staphylococcus aureus, which antibody is a functionally active variant antibody of a parent antibody that comprises a polyspecific binding site of the VH amino acid sequence of SEQ ID 20, and the VL amino acid sequence of SEQ ID 39, which functionally active variant antibody comprises at least one point mutation in any of the framework regions (FR) or constant domains, or complementarity determining regions (CDR1 to CDR6) in any of SEQ ID 20 or SEQ 39, and has an affinity to bind each of the toxins with a Kd of less than 10⁻⁸M, preferably less than 10⁻⁹M.

11. The antibody of claim 9, wherein
a) in VH CDR1 at position 5, the amino acid residue is selected from the group consisting of S, A, D, E, F, G, H, I, K, L, M, N, Q, R, T V, W and Y;
b) in VH CDR1 at position 7, the amino acid residue is selected from the group consisting of M, H, K, Q, R and W;
c) in VH CDR2 at position 3, the amino acid residue is selected from the group consisting of D and R;
d) in VH CDR2 at position 7, the amino acid residue is selected from the group consisting of S, A, D, E, F, H, K, M, N, Q, R, T, W and Y;
e) in VH CDR2 at position 9, the amino acid residue is selected from the group consisting of Y, F, K, L, Q and R;
f) in VH CDR3 at position 5, the amino acid residue is selected from the group consisting of G, A, D, F, H, I, M, N, R, S, T, V and Y;
g) in VH CDR3 at position 6, the amino acid residue is selected from the group consisting of H, E, Q and S;
h) in VH CDR3 at position 7, the amino acid residue is selected from the group consisting of G, A, D, E, H, I, M, N, Q, S, T, V and W; and/or
i) in VH CDR3 at position 8, the amino acid residue is selected from the group consisting of V, A, D, E, G, I, K, L, M, Q, R, S and T.

12. The antibody of claim 10, wherein
a) in VL CDR4 at position 7, the amino acid residue is selected from the group consisting of S, A, E, F, G, K, L, M, N, Q, R, W and Y;
b) in VL CDR5 at position 1, the amino acid residue is selected from the group consisting of A and G;
c) in VL CDR5 at position 3, the amino acid residue is selected from the group consisting of S, A, D, G, H, I, K, L, N, Q, R, T, V and W;
d) in VL CDR5 at position 4, the amino acid residue is selected from the group consisting of S, D, E, H, I, K, M, N, Q, R, T and V;
e) in VL CDR6 at position 3, the amino acid residue is selected from the group consisting of G, A, D, E, F, H, I, K, L, N, Q, R, S, T, V, W and Y;
f) in VL CDR6 at position 4, the amino acid residue is selected from the group consisting of Y, D, F, H, M, R and W;
g) in VL CDR6 at position 5, the amino acid residue is selected from the group consisting of V, A, D, E, F, G, H, I, K, L, M, N, Q, R, S, T, and W; and/or
h) in VL CDR6 at position 6, the amino acid residue is selected from the group consisting of F and W.

13. The antibody of claim 1, which has a cross-specificity to bind Hla and at least one of the F-components of the bi-component toxins, wherein the F-components are selected from the group consisting of HlgB, LukF and LukD, or any F-component of the cognate and non-cognate pairs of F and S components of gamma-hemolysins, PVL toxins and PVL-like toxins.

14. The antibody of claim 1, which is a full-length monoclonal antibody, an antibody fragment thereof comprising at least one antibody domain incorporating the binding site, or a fusion protein comprising at least one antibody domain incorporating the binding site.

15. A method for treating a subject at risk of or suffering from a S. aureus infection comprising administering to the subject an effective amount of the antibody of claim 1 to limit the infection in the subject, to ameliorate a disease condition resulting from said infection or to inhibit S. aureus disease pathogenesis.

16. A pharmaceutical preparation comprising the antibody according to claim 1 and a pharmaceutically acceptable carrier or excipient.

17. (canceled)

18. (canceled)

19. Isolated nucleic acid encoding an antibody according to claim 1.

20. A recombinant expression cassette or a plasmid comprising a coding sequence to express a light chain and/or heavy chain of an antibody according to claim 1.

21. A host cell comprising the expression cassette or the plasmid of claim 20.

22. A method of producing an antibody comprising cultivating or maintaining the host cell according to claim 21 under conditions to produce said antibody.

23. A method of producing functionally active antibody variants of a parent antibody which is any of the antibodies comprising a polyspecific binding site of the VH amino acid sequence of any of SEQ ID 20-31, and the VL amino acid sequence of SEQ ID 39, which method comprises engineering at least one point mutation in any of the framework regions (FR) or constant domains, or complementarity determining regions (CDR1 to CDR6) in any of SEQ ID 20-31 or SEQ 39 to obtain a variant antibody, and determining the functional activity of the variant antibody by any of
the affinity to bind each of Hla and at least one of the bi-component toxins of S. aureus with a Kd of less than 10⁻⁸M, preferably less than 10⁻⁹M, and/or
the binding of the variant antibody to Hla and/or the at least one of the bi-component toxins in competition with the parent antibody;
wherein upon determining the functional activity, the functionally active variants are selected for production by a recombinant production method.

24. A crystal formed by a Hla monomer that diffracts x-ray radiation to produce a diffraction pattern representing the three-dimensional structure of the Hla rim domain in contact with the antibody of claim 1, or a binding fragment thereof, having the following cell constants: 285.05 Å, 150.94 Å, 115.25 Å, space group P2₁2₁2, optionally with a deviation of between 0.00 Å and 2.00 Å.

25. A crystal formed by a LukD monomer that diffracts x-ray radiation to produce a diffraction pattern representing the three-dimensional structure of the LukD rim domain in contact with the antibody of claim 1, or a binding fragment thereof, having the following cell constants: 112.0 Å, 112.0 Å, 409.3 Å, space group H32, optionally with a deviation of between 0.00 Å and 2.00 Å.

26. The isolated paratope of an antibody of claim 1, or a binding molecule comprising said paratope.

27. An isolated conformational epitope recognized by the antibody of claim 1, characterized by a three-dimensional structure of the rim domain of Hla, LukD, LukF or HlgB.

28. The epitope of claim 27, characterized by a three-dimensional structure selected from the group consisting of
a) the three-dimensional Hla structure characterized by the structure coordinates of the contact amino acid residues 179-191, 194, 200, 269 and 271 of SEQ ID 54;
b) the three-dimensional LukF structure characterized by the structure coordinates of the contact amino acid residues 176-188, 191, 197 and 267 of SEQ ID 55, preferably with amino acid residues 176-179, 181-184, 186-188, 191, 197 and 267 of SE ID 58;
c) the three-dimensional LukD structure characterized by the structure coordinates of the contact amino acid residues 176-188, 191, 197 and 267 of SEQ ID 54, preferably with amino acid residues 176-179, 181-184, 186-188, 191, 197 and 267 of SEQ ID 62;
d) the three-dimensional HlgB structure characterized by the structure coordinates of the amino acid contact residues 177-189, 192, 198 and 268 of SEQ ID 56, preferably with amino acid residues 177-180, 182-185, 187-189, 192, 198 and 268 of SEQ ID 68,
e) the three-dimensional Hla rim domain structure of the crystal of definition 26;
f) the three-dimensional LukD rim domain structure of the crystal of definition 27; and
g) a three-dimensional structure which is a homolog of any of a) to f) wherein said homolog comprises a binding site that has a root mean square deviation from backbone atoms of contact amino acid residues of between 0.00 Å and 2.00 Å.

29. The epitope of claim 27, which is bound by a binding molecule.

30. A binding molecule which is specifically binds to the epitope of claim 27, selected from the group consisting of a protein, a peptide, a peptidomimetic, a nucleic acid, a carbohydrate, a lipid, an oligopeptide, an aptamer and a small molecule compound, preferably an antibody, an antibody fragment thereof comprising at least one antibody domain incorporating the binding site, or a fusion protein comprising at least one antibody domain incorporating the binding site, wherein the binding molecule is a polyspecific binder that binds to Hla and at least one of the bi-component toxins of S. aureus.

31. A screening method or assay for identifying a binder which specifically binds to the epitope of claim 28, comprising the steps of:
bringing a candidate compound into contact with the three-dimensional structure as defined in claim 28; and
assessing binding between the candidate compound and the three-dimensional structure; wherein binding between the candidate compound and the three-dimensional structure identifies the candidate compound as a polyspecific binder that binds to Hla and at least one of the bi-component toxins of S. aureus.

32. An immunogen comprising:
a) an epitope of claim 27;
b) optionally epitopes not natively associated with said epitope of (a); and
c) a pharmaceutically acceptable carrier.

33. Immunogen according to claim 32 in a vaccine formulation comprising adjuvant substances.

34. A method of treating a subject comprising administering to the subject an effective amount of the immunogen of claim 32 to protect the subject from an S. aureus infection, to prevent a disease condition resulting from said infection or to inhibit S. aureus pneumonia pathogenesis.

35. (canceled)

36. A method for ex vivo diagnosis of a systemic infection with S. aureus in a subject comprising contacting a sample of body fluid of the subject with the antibody of claim 1, allowing a specific immune reaction with the antibody as immunoreagent, wherein the specific immune reaction determines the systemic infection with S. aureus.

37. A composition comprising the antibody of claim 1 with a label and/or a further diagnostic reagent with a label.

38. A method of inducing a protective immune response in a subject, comprising administering to the subject an effective amount of the immunogen of claim 32.


SEQUENCE LISTING (80 sequences)

SEQ ID NO: 1 - protein, 10 aa, artificial
  partial antibody sequence
YSISSGMGWG

SEQ ID NO: 2 - protein, 16 aa, artificial
  partial antibody sequence
SIDQRGSTYYNPSLKS

SEQ ID NO: 3 - protein, 12 aa, artificial
  partial antibody sequence
ARDAGHGVDMDV

SEQ ID NO: 4 - protein, 10 aa, artificial
  partial antibody sequence
YPISSGMGWG

SEQ ID NO: 5 - protein, 10 aa, artificial
  partial antibody sequence
YSISSGMGWD

SEQ ID NO: 6 - protein, 16 aa, artificial
  partial antibody sequence
SVDQRGSTYYNPSLKS

SEQ ID NO: 7 - protein, 16 aa, artificial
  partial antibody sequence
RIDQRGSTYYNPSLKS

SEQ ID NO: 8 - protein, 16 aa, artificial
  partial antibody sequence
RVDQRGSTYYNPSLKS

SEQ ID NO: 9 - protein, 16 aa, artificial
  partial antibody sequence
SIDQRGSTYYNPSLEG

SEQ ID NO: 10 - protein, 16 aa, artificial
  partial antibody sequence
SIDQRGSTYYNPPLES

SEQ ID NO: 11 - protein, 12 aa, artificial
  partial antibody sequence
ARDAGHGADMDV

SEQ ID NO: 12 - protein, 12 aa, artificial
  partial antibody sequence
ARDAGHAVDMDV

SEQ ID NO: 13 - protein, 26 aa, artificial
  partial antibody sequence
QVQLQESGPGLVKPSETLSLTCAVSG

SEQ ID NO: 14 - protein, 26 aa, artificial
  partial antibody sequence
QVQLQESGPGLVRPSETLSLTCAVSG

SEQ ID NO: 15 - protein, 14 aa, artificial
  partial antibody sequence
WIRQPPGKGLEWIG

SEQ ID NO: 16 - protein, 14 aa, artificial
  partial antibody sequence
WIRQPPGKGREWIG

SEQ ID NO: 17 - protein, 30 aa, artificial
  partial antibody sequence
RVTISVDTSKNQFSLKLSSVTAADTAVYYC

SEQ ID NO: 18 - protein, 30 aa, artificial
  partial antibody sequence
RATISVDTSKNQFSLKLSSVTAADTAVYYC

SEQ ID NO: 19 - protein, 11 aa, artificial
  partial antibody sequence
WGKGTTVTVSS

SEQ ID NO: 20 - protein, 119 aa, artificial
  VH-Sequence
QVQLQESGPGLVKPSETLSLTCAVSGYSISSGMGWGWIRQPPGKGLEWIGSIDQRGSTYYNPSLKSRVTISVDTSKNQFSLKLSSVTAADTAVYYCARDAGHGVDMDVWGKGTTVTVSS

SEQ ID NO: 21 - protein, 119 aa, artificial
  VH-Sequence
QVQLQESGPGLVKPSETLSLTCAVSGYSISSGMGWGWIRQPPGKGLEWIGSVDQRGSTYYNPSLKSRVTISVDTSKNQFSLKLSSVTAADTAVYYCARDAGHGADMDVWGKGTTVTVSS

SEQ ID NO: 22 - protein, 119 aa, artificial
  VH-Sequence
QVQLQESGPGLVKPSETLSLTCAVSGYPISSGMGWGWIRQPPGKGLEWIGRIDQRGSTYYNPSLKSRATISVDTSKNQFSLKLSSVTAADTAVYYCARDAGHGVDMDVWGKGTTVTVSS

SEQ ID NO: 23 - protein, 119 aa, artificial
  VH-Sequence
QVQLQESGPGLVKPSETLSLTCAVSGYSISSGMGWGWIRQPPGKGLEWIGRVDQRGSTYYNPSLKSRVTISVDTSKNQFSLKLSSVTAADTAVYYCARDAGHGVDMDVWGKGTTVTVSS

SEQ ID NO: 24 - protein, 119 aa, artificial
  VH-Sequence
QVQLQESGPGLVRPSETLSLTCAVSGYSISSGMGWGWIRQPPGKGLEWIGSIDQRGSTYYNPSLKSRVTISVDTSKNQFSLKLSSVTAADTAVYYCARDAGHAVDMDVWGKGTTVTVSS

SEQ ID NO: 25 - protein, 119 aa, artificial
  VH-Sequence
QVQLQESGPGLVKPSETLSLTCAVSGYSISSGMGWDWIRQPPGKGREWIGSIDQRGSTYYNPSLEGRATISVDTSKNQFSLKLSSVTAADTAVYYCARDAGHGVDMDVWGKGTTVTVSS

SEQ ID NO: 26 - protein, 119 aa, artificial
  VH-Sequence
QVQLQESGPGLVKPSETLSLTCAVSGYSISSGMGWDWIRQPPGKGLEWIGSIDQRGSTYYNPPLESRVTISVDTSKNQFSLKLSSVTAADTAVYYCARDAGHGVDMDVWGKGTTVTVSS

SEQ ID NO: 27 - protein, 119 aa, artificial
  VH-Sequence
EVQLQESGPGLVRPSETLSLTCAVSGYSISSGMGWGWIRQPPGKGLEWIGSIDQRGSTYYNPSLKSRVTISVDTSKNQFSLKLSSVTAADTAVYYCARDAGHAVDMDVWGKGTTVTVSS

SEQ ID NO: 28 - protein, 119 aa, artificial
  VH-Sequence
QVQLQESGPGLVKPSETLSLTCAVSGYSISSGMGWGWIRQPPGKGLEWIGSIDQRGSTYYNPSLKSRVTISVDTSKNQFSLKLSSVTAADTAVYYCARDAGHAVDMDVWGKGTTVTVSS

SEQ ID NO: 29 - protein, 119 aa, artificial
  VH-Sequence
QVQLQESGPGLVKPSETLSLTCAVSGYSISSGMGWDWIRQPPGKGLEWIGSIDQRGSTYYNPSLEGRVTISVDTSKNQFSLKLSSVTAADTAVYYCARDAGHGVDMDVWGKGTTVTVSS

SEQ ID NO: 30 - protein, 119 aa, artificial
  VH-Sequence
QVQLQESGPGLVKPSETLSLTCAVSGYSISSGMGWDWIRQPPGKGREWIGSIDQRGSTYYNPSLEGRVTISVDTSKNQFSLKLSSVTAADTAVYYCARDAGHGVDMDVWGKGTTVTVSS

SEQ ID NO: 31 - protein, 119 aa, artificial
  VH-Sequence
QVQLQESGPGLVKPSETLSLTCAVSGYSISSGMGWDWIRQPPGKGLEWIGSIDQRGSTYYNPSLEGRATISVDTSKNQFSLKLSSVTAADTAVYYCARDAGHGVDMDVWGKGTTVTVSS

SEQ ID NO: 32 - protein, 11 aa, artificial
  partial antibody sequence
RASQGISRWLA

SEQ ID NO: 33 - protein, 7 aa, artificial
  partial antibody sequence
AASSLQS

SEQ ID NO: 34 - protein, 9 aa, artificial
  partial antibody sequence
QQGYVFPLT

SEQ ID NO: 35 - protein, 23 aa, artificial
  partial antibody sequence
DIQMTQSPSSVSASVGDRVTITC

SEQ ID NO: 36 - protein, 15 aa, artificial
  partial antibody sequence
WYQQKPGKAPKLLIY

SEQ ID NO: 37 - protein, 32 aa, artificial
  partial antibody sequence
GVPSRFSGSGSGTDFTLTISSLQPEDFATYYC

SEQ ID NO: 38 - protein, 10 aa, artificial
  partial antibody sequence
FGGGTKVEIK

SEQ ID NO: 39 - protein, 107 aa, artificial
  VL-Sequence
DIQMTQSPSSVSASVGDRVTITCRASQGISRWLAWYQQKPGKAPKLLIYAASSLQSGVPSRFSGSGSGTDFTLTISSLQPEDFATYYCQQGYVFPLTFGGGTKVEIK

SEQ ID NO: 40 - protein, 468 aa, artificial
  HC-Sequence
MELGLCWVFLVAILEGVQCQVQLQESGPGLVKPSETLSLTCAVSGYSISSGMGWGWIRQPPGKGLEWIGSIDQRGSTYYNPSLKSRVTISVDTSKNQFSLKLSSVTAADTAVYYCARDAGHGVDMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 41 - protein, 458 aa, artificial
  HC-Sequence
MELGLCWVFLVAILEGVQCQVQLQESGPGLVKPSETLSLTCAVSGYSISSGMGWGWIRQPPGKGLEWIGSVDQRGSTYYNPSLKSRVTISVDTSKNQFSLKLSSVTAADTAVYYCARDAGHGADMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYT

SEQ ID NO: 42 - protein, 456 aa, artificial
  HC-Sequence
MELGLCWVFLVAILEGVQCQVQLQESGPGLVKPSETLSLTCAVSGYPISSGMGWGWIRQPPGKGLEWIGRIDQRGSTYYNPSLKSRATISVDTSKNQFSLKLSSVTAADTAVYYCARDAGHGVDMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNH

SEQ ID NO: 43 - protein, 456 aa, artificial
  HC-Sequence
MELGLCWVFLVAILEGVQCQVQLQESGPGLVKPSETLSLTCAVSGYSISSGMGWGWIRQPPGKGLEWIGRVDQRGSTYYNPSLKSRVTISVDTSKNQFSLKLSSVTAADTAVYYCARDAGHGVDMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNH

SEQ ID NO: 44 - protein, 468 aa, artificial
  HC-Sequence
MELGLCWVFLVAILEGVQCQVQLQESGPGLVRPSETLSLTCAVSGYSISSGMGWGWIRQPPGKGLEWIGSIDQRGSTYYNPSLKSRVTISVDTSKNQFSLKLSSVTAADTAVYYCARDAGHAVDMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 45 - protein, 468 aa, artificial
  HC-Sequence
MELGLCWVFLVAILEGVQCQVQLQESGPGLVKPSETLSLTCAVSGYSISSGMGWDWIRQPPGKGREWIGSIDQRGSTYYNPSLEGRATISVDTSKNQFSLKLSSVTAADTAVYYCARDAGHGVDMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 46 - protein, 468 aa, artificial
  HC-Sequence
MELGLCWVFLVAILEGVQCQVQLQESGPGLVKPSETLSLTCAVSGYSISSGMGWDWIRQPPGKGLEWIGSIDQRGSTYYNPPLESRVTISVDTSKNQFSLKLSSVTAADTAVYYCARDAGHGVDMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 47 - protein, 467 aa, artificial
  HC-Sequence
MELGLCWVFLVAILEGVQCEVQLQESGPGLVRPSETLSLTCAVSGYSISSGMGWGWIRQPPGKGLEWIGSIDQRGSTYYNPSLKSRVTISVDTSKNQFSLKLSSVTAADTAVYYCARDAGHAVDMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 48 - protein, 468 aa, artificial
  HC-Sequence
MELGLCWVFLVAILEGVQCQVQLQESGPGLVKPSETLSLTCAVSGYSISSGMGWGWIRQPPGKGLEWIGSIDQRGSTYYNPSLKSRVTISVDTSKNQFSLKLSSVTAADTAVYYCARDAGHAVDMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 49 - protein, 468 aa, artificial
  HC-Sequence
MELGLCWVFLVAILEGVQCQVQLQESGPGLVKPSETLSLTCAVSGYSISSGMGWDWIRQPPGKGLEWIGSIDQRGSTYYNPSLEGRVTISVDTSKNQFSLKLSSVTAADTAVYYCARDAGHGVDMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 50 - protein, 468 aa, artificial
  HC-Sequence
MELGLCWVFLVAILEGVQCQVQLQESGPGLVKPSETLSLTCAVSGYSISSGMGWDWIRQPPGKGREWIGSIDQRGSTYYNPSLEGRVTISVDTSKNQFSLKLSSVTAADTAVYYCARDAGHGVDMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 51 - protein, 468 aa, artificial
  HC-Sequence
MELGLCWVFLVAILEGVQCQVQLQESGPGLVKPSETLSLTCAVSGYSISSGMGWDWIRQPPGKGLEWIGSIDQRGSTYYNPSLEGRATISVDTSKNQFSLKLSSVTAADTAVYYCARDAGHGVDMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 52 - protein, 233 aa, artificial
  LC-Sequence
MELGLCWVFLVAILEGVQCDIQMTQSPSSVSASVGDRVTITCRASQGISRWLAWYQQKPGKAPKLLIYAASSLQSGVPSRFSGSGSGTDFTLTISSLQPEDFATYYCQQGYVFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 53 - DNA, 879 bp, Staphylococcus aureus
gccgacagcgacatcaacatcaaaacgggcacgacggacattggctcaaatacgacggtgaaaacgggcgatctggttacctatgacaaagaaaacggcatgcataaaaaagtgttttatagtttcatcgatgacaaaaaccacaacaaaaaactgctggtcattcgtaccaaaggcacgatcgcaggccagtatcgcgtgtacagcgaagaaggcgctaataaatcaggtctggcatggccgtcggcttttaaagttcagctgcaactgccggataacgaagtcgcgcaaattagcgactattacccgcgtaactctatcgataccaaagaatacatgtctaccctgacgtacggcttcaacggtaatgttaccggcgatgacacgggtaaaattggcggtctgatcggcgccaacgtgagcattggtcataccctgaaatatgttcagccggactttaaaaccatcctggaatctccgacggataaaaaagtgggctggaaagttatcttcaacaacatggttaaccagaactggggtccgtatgatcgtgactcatggaacccggtctacggcaatcaactgtttatgaaaacccgcaacggttcgatgaaagcggccgataacttcctggacccgaataaagcgagctctctgctgagttccggctttagtccggacttcgcgaccgtgattacgatggatcgcaaagcctccaaacagcaaaccaatattgatgtcatctatgaacgtgtgcgcgatgactaccagctgcactggaccagcacgaactggaaaggtaccaatacgaaagataaatggattgaccgctcctcggaacgctacaaaattgactgggaaaaagaagaaatgacgaac

SEQ ID NO: 54 - protein, 293 aa, Staphylococcus aureus
ADSDINIKTGTTDIGSNTTVKTGDLVTYDKENGMHKKVFYSFIDDKNHNKKLLVIRTKGTIAGQYRVYSEEGANKSGLAWPSAFKVQLQLPDNEVAQISDYYPRNSIDTKEYMSTLTYGFNGNVTGDDTGKIGGLIGANVSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWIDRSSERYKIDWEKEEMTN

SEQ ID NO: 55 - DNA, 852 bp, Staphylococcus aureus
gacaacaacattgaaaacattggtgatggcgcagaagtggtgaaacgcacggaagatacctcaagcgataaatggggtgtgacgcagaacattcagttcgatttcgtcaaagacaaaaaatacaacaaagatgcactgattctgaaaatgcaaggctttatcaacagcaaaaccacgtactacaactacaaaaacaccgaccatatcaaagctatgcgttggccgttccagtacaatatcggtctgaaaacgaacgatccgaatgttgacctgatcaactacctgccgaaaaacaaaatcgattcagtgaacgtttcgcaaaccctgggctacaatatcggcggtaactttaatagtggcccgtccaccggcggtaacggtagcttcaactactctaaaacgatcagttacaaccagcaaaactacatctctgaagtcgaacgtcagaacagcaaatctgtgcaatggggcattaaagcgaattcctttatcacctcactgggcaaaatgtcgggtcatgatccgaacctgtttgtgggttataaaccgtacagccagaacccgcgcgattatttcgttccggacaatgaactgccgccgctggtccattctggctttaacccgagtttcattgcaaccgtgagccacgaaaaaggctcgggtgataccagcgaatttgaaatcacgtatggtcgcaatatggacgttacccatgcgacgcgtcgcaccacgcactatggcaactcctacctggaaggttcacgtattcacaatgccttcgttaaccgcaattacacggtgaaatacgaagtcaactggaaaacgcacgaaatcaaagtgaaaggtcataac

SEQ ID NO: 56 - protein, 284 aa, Staphylococcus aureus
DNNIENIGDGAEVVKRTEDTSSDKWGVTQNIQFDFVKDKKYNKDALILKMQGFINSKTTYYNYKNTDHIKAMRWPFQYNIGLKTNDPNVDLINYLPKNKIDSVNVSQTLGYNIGGNFNSGPSTGGNGSFNYSKTISYNQQNYISEVERQNSKSVQWGIKANSFITSLGKMSGHDPNLFVGYKPYSQNPRDYFVPDNELPPLVHSGFNPSFIATVSHEKGSGDTSEFEITYGRNMDVTHATRRTTHYGNSYLEGSRIHNAFVNRNYTVKYEVNWKTHEIKVKGHN

SEQ ID NO: 57 - DNA, 903 bp, Staphylococcus aureus
gcgcagcacatcacgccggtctccgaaaaaaaagttgacgacaaaatcaccctgtataaaacgacggccacgagcgactctgacaaactgaaaatttctcagatcctgaccttcaacttcatcaaagataaaagttacgataaagacacgctgattctgaaagcggccggtaacatctattctggctacaccaaaccgaatccgaaagacacgatcagctctcaattctactggggttccaaatacaacatctcaatcaacagtgattccaacgactccgtcaatgtggttgattatgcaccgaaaaaccagaatgaagaattccaagtccagcaaaccgtgggctatagttacggcggtgacattaacatctcgaatggtctgagcggcggtggcaacggctcaaaatcgttcagcgaaacgatcaactacaaacaggaatcttaccgtaccagtctggataaacgcacgaatttcaagaaaattggttgggacgttgaagcgcataaaatcatgaacaatggttggggcccgtatggccgtgattcttatcacagtacctacggtaacgaaatgtttctgggctcccgccagtcaaacctgaatgccggtcaaaatttcctggaataccataaaatgccggttctgagccgtggtaactttaatccggaattcattggcgtcctgtcgcgcaaacagaacgcagcgaaaaaatctaaaatcaccgtgacgtatcagcgtgaaatggatcgctacaccaacttttggaatcaactgcattggatcggcaacaactacaaagatgaaaaccgtgccacccacacgagcatctacgaagttgactgggaaaaccacacggtgaaactgattgatacccaaagtaaagaaaaaaacccgatgtcg

SEQ ID NO: 58 - protein, 301 aa, Staphylococcus aureus
AQHITPVSEKKVDDKITLYKTTATSDSDKLKISQILTFNFIKDKSYDKDTLILKAAGNIYSGYTKPNPKDTISSQFYWGSKYNISINSDSNDSVNVVDYAPKNQNEEFQVQQTVGYSYGGDINISNGLSGGGNGSKSFSETINYKQESYRTSLDKRTNFKKIGWDVEAHKIMNNGWGPYGRDSYHSTYGNEMFLGSRQSNLNAGQNFLEYHKMPVLSRGNFNPEFIGVLSRKQNAAKKSKITVTYQREMDRYTNFWNQLHWIGNNYKDENRATHTSIYEVDWENHTVKLIDTQSKEKNPMS

SEQ ID NO: 59 - DNA, 849 bp, Staphylococcus aureus
aatacgaatatcgaaaatatcggcgacggcgcagaagttatcaaacgcacggaagatgtcagcagcaaaaaatggggtgttacgcagaatgttcagttcgatttcgtcaaagacaaaaaatacaacaaagatgcactgattgtgaaaatgcaaggctttatcaattctcgtaccagtttctccgacgttaaaggcagtggttatgaactgacgaaacgcatgatttggccgtttcagtacaacatcggtctgaccacgaaagatccgaacgtttccctgatcaactacctgccgaaaaacaaaatcgaaaccacggacgtcggccagaccctgggttacaacattggcggtaattttcaaagcgctccgtctatcggcggtaacggctcattcaattactcgaaaaccattagctatacgcagaaaagttacgtgtccgaagttgataaacaaaactcaaaatcggtcaaatggggcgtgaaagcgaacgaatttgtcaccccggatggtaaaaaatctgcccatgaccgttacctgtttgtgcagtcgccgaatggtccgacgggtagcgcacgtgaatactttgccccggataatcagctgccgccgctggtgcaatctggctttaacccgagtttcattaccacgctgagccatgaaaaaggcagctctgatacctccgaattcgaaatttcatatggtcgtaatctggacatcacctacgcaacgctgtttccgcgtaccggtatctatgcagaacgcaaacacaacgcttttgttaaccgcaatttcgttgtccgctacgaagtgaactggaaaacccatgaaatcaaagtgaaaggccataac

SEQ ID NO: 60 - protein, 283 aa, Staphylococcus aureus
NTNIENIGDGAEVIKRTEDVSSKKWGVTQNVQFDFVKDKKYNKDALIVKMQGFINSRTSFSDVKGSGYELTKRMIWPFQYNIGLTTKDPNVSLINYLPKNKIETTDVGQTLGYNIGGNFQSAPSIGGNGSFNYSKTISYTQKSYVSEVDKQNSKSVKWGVKANEFVTPDGKKSAHDRYLFVQSPNGPTGSAREYFAPDNQLPPLVQSGFNPSFITTLSHEKGSSDTSEFEISYGRNLDITYATLFPRTGIYAERKHNAFVNRNFVVRYEVNWKTHEIKVKGHN

SEQ ID NO: 61 - DNA, 903 bp, Staphylococcus aureus
gcccaacacattacgccggtctcggaaaaaaaagtggatgacaaaatcacgctgtataaaacgacggcaacctcagataacgacaaactgaacattagtcagatcctgaccttcaacttcatcaaagataaatcctacgataaagacacgctggtgctgaaagcggccggcaacattaattcaggttacaaaaaaccgaacccgaaagactataattactcgcagttttattggggcggtaaatacaacgtcagcgtgagctctgaatctaacgatgcagtcaatgtggttgactatgctccgaaaaaccagaatgaagaatttcaagtgcagcaaaccctgggctatagctacggcggtgatattaacatctcaaatggcctgtcgggcggtctgaacggttcgaaaagcttctctgaaaccatcaactacaaacaggaaagctaccgtaccacgattgatcgcaaaacgaaccataaatctatcggctggggtgttgaagcgcacaaaattatgaacaatggctggggtccgtatggccgtgattcctatgacccgacctacggtaatgaactgtttctgggcggtcgccagagttcctcaaacgcgggccaaaatttcctgccgacgcatcagatgccgctgctggcacgtggtaactttaatccggaattcatcagtgtgctgtcccacaaacaaaacgataccaaaaaatctaaaatcaaagttacgtatcaacgtgaaatggaccgctacaccaaccagtggaatcgcctgcattgggttggtaacaactacaaaaaccagaacaccgttacgttcacctctacgtacgaagtcgattggcaaaaccatacggtcaaactgattggcacggacagcaaagaaacgaacccgggcgtc

SEQ ID NO: 62 - protein, 301 aa, Staphylococcus aureus
AQHITPVSEKKVDDKITLYKTTATSDNDKLNISQILTFNFIKDKSYDKDTLVLKAAGNINSGYKKPNPKDYNYSQFYWGGKYNVSVSSESNDAVNVVDYAPKNQNEEFQVQQTLGYSYGGDINISNGLSGGLNGSKSFSETINYKQESYRTTIDRKTNHKSIGWGVEAHKIMNNGWGPYGRDSYDPTYGNELFLGGRQSSSNAGQNFLPTHQMPLLARGNFNPEFISVLSHKQNDTKKSKIKVTYQREMDRYTNQWNRLHWVGNNYKNQNTVTFTSTYEVDWQNHTVKLIGTDSKETNPGV

SEQ ID NO: 63 - DNA, 840 bp, Staphylococcus aureus
gaaaacaaaatcgaagacatcggccaaggtgctgaaatcatcaaacgcacgcaagacatcacgagtaaacgcctggcaatcacgcagaatattcagttcgatttcgtgaaagacaaaaaatacaacaaagatgcactggtggttaaaatgcaaggctttatcagctctcgtaccacgtacagcgatctgaaaaaatatccgtacattaaacgcatgatctggccgttccagtacaacatcagtctgaaaaccaaagattccaacgtggacctgattaattacctgccgaaaaacaaaatcgatagtgcggacgtttcccagaaactgggctataacattggcggtaattttcaatcagccccgtcgatcggcggtagtggttccttcaattactcaaaaaccatctcgtacaaccagaaaaattacgttacggaagtcgaaagccaaaactctaaaggcgtgaaatggggtgttaaagcgaattcatttgtcaccccgaacggccaggtgtcggcgtatgatcagtacctgtttgcacaagacccgacgggtccggcagcacgtgattatttcgttccggacaatcagctgccgccgctgattcaaagcggctttaacccgtctttcatcaccacgctgtcccatgaacgtggcaaaggtgataaaagcgaatttgaaattacctatggtcgcaacatggatgcaacctatgcttacgttacgcgtcatcgcctggcagtcgatcgtaaacacgacgctttcaaaaaccgcaatgtcaccgtgaaatacgaagtcaactggaaaacgcacgaagtcaaaatcaaatcaatcaccccgaaa

SEQ ID NO: 64 - protein, 280 aa, Staphylococcus aureus
ENKIEDIGQGAEIIKRTQDITSKRLAITQNIQFDFVKDKKYNKDALVVKMQGFISSRTTYSDLKKYPYIKRMIWPFQYNISLKTKDSNVDLINYLPKNKIDSADVSQKLGYNIGGNFQSAPSIGGSGSFNYSKTISYNQKNYVTEVESQNSKGVKWGVKANSFVTPNGQVSAYDQYLFAQDPTGPAARDYFVPDNQLPPLIQSGFNPSFITTLSHERGKGDKSEFEITYGRNMDATYAYVTRHRLAVDRKHDAFKNRNVTVKYEVNWKTHEVKIKSITPK

SEQ ID NO: 65 - DNA, 858 bp, Staphylococcus aureus
gcaaacgacacggaagacatcggcaaaggttcagacatcgaaatcatcaaacgcacggaagacaaaacgagcaataaatggggtgtgacccagaacattcaattcgatttcgtgaaagacaaaaaatacaataaagatgcgctgattctgaaaatgcagggctttatcagctctcgtaccacgtactacaactacaagaaaaccaaccatgttaaagccatgcgctggccgttccaatacaacatcggtctgaaaacgaatgacaaatatgtcagtctgattaactacctgccgaaaaataaaatcgaatcgaccaacgtgagccagacgctgggctataacattggcggtaattttcaatccgcaccgtcactgggcggtaacggttcattcaattactcaaaatcgatcagctatacccagcaaaactacgtgtctgaagttgaacagcaaaattctaaaagtgtcctgtggggcgtgaaagcgaatagctttgccacggaatctggtcagaaaagtgcatttgattccgacctgttcgtgggctataaaccgcattcaaaagatccgcgtgactacttcgtgccggattcggaactgccgccgctggttcagtcaggttttaacccgtcgttcattgctaccgttagtcacgaaaaaggcagttccgatacctccgaatttgaaattacgtatggtcgtaatatggacgtcacccatgcaatcaaacgcagcacgcactatggcaactcttacctggatggtcatcgtgttcacaatgcttttgtcaaccgcaattatacggtgaaatacgaagtcaactggaaaacgcacgaaatcaaagtcaaaggtcaaaac

SEQ ID NO: 66 - protein, 286 aa, Staphylococcus aureus
ANDTEDIGKGSDIEIIKRTEDKTSNKWGVTQNIQFDFVKDKKYNKDALILKMQGFISSRTTYYNYKKTNHVKAMRWPFQYNIGLKTNDKYVSLINYLPKNKIESTNVSQTLGYNIGGNFQSAPSLGGNGSFNYSKSISYTQQNYVSEVEQQNSKSVLWGVKANSFATESGQKSAFDSDLFVGYKPHSKDPRDYFVPDSELPPLVQSGFNPSFIATVSHEKGSSDTSEFEITYGRNMDVTHAIKRSTHYGNSYLDGHRVHNAFVNRNYTVKYEVNWKTHEIKVKGQN

SEQ ID NO: 67 - DNA, 900 bp, Staphylococcus aureus
gcggaaggcaaaattaccccggtctcggtgaaaaaagttgacgacaaagtgacgctgtataaaacgacggccacggctgattcggataaatttaaaattagccagatcctgaccttcaacttcatcaaagataaatcttacgataaagacaccctggtgctgaaagcaacgggcaacatcaatagcggttttgttaaaccgaacccgaatgattacgacttctcaaaactgtattggggcgcaaaatacaatgtttcgattagctctcagagtaacgattccgtcaatgtggttgactatgctccgaaaaaccaaaatgaagaatttcaggtgcaaaacaccctgggttacacgttcggcggtgatatttcaatctcgaatggcctgagtggcggtctgaacggtaataccgcgttttccgaaacgattaactataaacaggaaagctaccgtaccacgctgtctcgcaacaccaattataaaaatgtcggctggggtgtggaagcccataaaatcatgaacaatggctggggtccgtatggccgtgactcctttcacccgacgtacggcaacgaactgttcctggcaggtcgccagagttccgcatatgcaggtcaaaattttattgcccagcatcaaatgccgctgctgagccgttctaactttaatccggaattcctgtcagtcctgtcgcaccgccaggatggcgcgaaaaaatctaaaatcaccgttacgtaccagcgtgaaatggacctgtaccaaatccgctggaacggcttctattgggcaggtgctaactacaaaaacttcaaaacccgtacgttcaaatctacctatgaaatcgattgggaaaaccacaaagtcaaactgctggacacgaaagaaacggaaaataataaa

SEQ ID NO: 68 - protein, 300 aa, Staphylococcus aureus
AEGKITPVSVKKVDDKVTLYKTTATADSDKFKISQILTFNFIKDKSYDKDTLVLKATGNINSGFVKPNPNDYDFSKLYWGAKYNVSISSQSNDSVNVVDYAPKNQNEEFQVQNTLGYTFGGDISISNGLSGGLNGNTAFSETINYKQESYRTTLSRNTNYKNVGWGVEAHKIMNNGWGPYGRDSFHPTYGNELFLAGRQSSAYAGQNFIAQHQMPLLSRSNFNPEFLSVLSHRQDGAKKSKITVTYQREMDLYQIRWNGFYWAGANYKNFKTRTFKSTYEIDWENHKVKLLDTKETENNK

SEQ ID NO: 69 - DNA, 972 bp, Staphylococcus aureus
aactcggctcataaagatagtcaggatcaaaataaaaaagaacacgtggataaatcacaacagaaagataaacgcaatgtcaccaataaagataaaaatagcaccgcaccggatgacattggcaaaaacggtaaaatcaccaaacgtaccgaaacggtgtatgatgaaaaaacgaatattctgcagaacctgcaatttgatttcatcgatgacccgacctacgacaaaaatgtgctgctggttaaaaaacagggcagcattcattctaacctgaaattcgaaagtcacaaagaagagaaaaactccaactggctgaaatatccgtcagaataccatgtcgatttccaggtgaaacgtaatcgcaaaaccgaaattctggaccaactgccgaaaaacaaaatcagtaccgccaaagttgatagtacgttttcctatagctctggcggtaaattcgactctaccaaaggcatcggtcgtacgagttccaactcatactcgaaaaccatctcgtacaaccagcaaaactacgatacgatcgcaagcggcaaaaacaataactggcatgttcactggtctgtcattgctaacgatctgaaatatggcggtgaagttaaaaatcgcaacgacgaactgctgttttaccgtaatacccgcatcgcgacggtcgaaaacccggaactgtcattcgcgtcgaaatatcgttacccggccctggtgcgctccggttttaatccggaattcctgacctacctgagcaacgaaaaatctaacgaaaaaacgcagttcgaagtcacctatacgcgtaatcaagatattctgaaaaaccgtccgggcattcactacgcaccgccgatcctggagaaaaacaaagatggtcagcgcctgatcgtgacctatgaagttgactggaaaaacaaaaccgtgaaagtggtggacaaatactcggacgacaataaaccgtacaaagaaggt

SEQ ID NO: 70 - protein, 324 aa, Staphylococcus aureus
NSAHKDSQDQNKKEHVDKSQQKDKRNVTNKDKNSTAPDDIGKNGKITKRTETVYDEKTNILQNLQFDFIDDPTYDKNVLLVKKQGSIHSNLKFESHKEEKNSNWLKYPSEYHVDFQVKRNRKTEILDQLPKNKISTAKVDSTFSYSSGGKFDSTKGIGRTSSNSYSKTISYNQQNYDTIASGKNNNWHVHWSVIANDLKYGGEVKNRNDELLFYRNTRIATVENPELSFASKYRYPALVRSGFNPEFLTYLSNEKSNEKTQFEVTYTRNQDILKNRPGIHYAPPILEKNKDGQRLIVTYEVDWKNKTVKVVDKYSDDNKPYKEG

SEQ ID NO: 71 - DNA, 927 bp, Staphylococcus aureus
aaaatcaacagcgaaatcaaacaagtcagcgaaaaaaatctggatggcgatacgaaaatgtacacgcgcacggcaaccacgagcgattcgcagaaaaacatcacccagagcctgcaatttaatttcctgaccgaaccgaactacgataaagaaacggtgttcatcaaagcaaaaggcaccatcggctcaggtctgcgtattctggacccgaatggctactggaactcgaccctgcgctggccgggtagctattctgtgagtattcagaatgttgatgacaacaataacaccaacgttacggattttgctccgaaaaatcaagatgaaagccgtgaagtcaaatatacctacggctataaaacgggcggtgatttctctatcaatcgcggcggtctgaccggtaatattacgaaagaatcgaactatagcgaaaccatctcctaccagcaaccgtcatatcgtaccctgctggatcagtccacgtcacataaaggcgttggttggaaagtcgaagcgcacctgatcaataacatgggccatgatcacacccgtcaactgacgaatgatagcgacaaccgcacgaaatctgaaatttttagtctgacccgcaatggtaacctgtgggcgaaagataacttcacgccgaaagacaaaatgccggtcaccgtgtccgaaggctttaatccggaattcctggccgttatgtctcatgataaaaaagacaaaggtaaaagtcagttcgtggttcactacaaacgttccatggatgaattcaaaatcgactggaaccgccatggcttctggggttactggagcggtgaaaaccacgtcgataaaaaagaagaaaaactgtctgcactgtatgaagtggactggaaaacccacaatgtcaaattcgtgaaagttctgaatgataatgaaaaaaaa

SEQ ID NO: 72 - protein, 309 aa, Staphylococcus aureus
KINSEIKQVSEKNLDGDTKMYTRTATTSDSQKNITQSLQFNFLTEPNYDKETVFIKAKGTIGSGLRILDPNGYWNSTLRWPGSYSVSIQNVDDNNNTNVTDFAPKNQDESREVKYTYGYKTGGDFSINRGGLTGNITKESNYSETISYQQPSYRTLLDQSTSHKGVGWKVEAHLINNMGHDHTRQLTNDSDNRTKSEIFSLTRNGNLWAKDNFTPKDKMPVTVSEGFNPEFLAVMSHDKKDKGKSQFVVHYKRSMDEFKIDWNRHGFWGYWSGENHVDKKEEKLSALYEVDWKTHNVKFVKVLNDNEKK

SEQ ID NO: 73 - DNA, 966 bp, Staphylococcus aureus
gcaaacaaggactcccaggaccagaccaaaaaagaacacgtcgataaagcacagcagaaagaaaagcgtaatgtcaacgataaagataaaaataccccgggcccggatgacattggcaaaaacggcaaggttaccaaacgtaccgtcagtgaatatgacaaagaaaccaatattctgcagaacctgcaatttgatttcatcgatgacccgacgtacgacaaaaatgtgctgctggttaaaaagcaaggtagtatccattccaacctgaagtttgaaagccaccgtaatgaaaccaacgcgagttggctgaaatatccgtccgaataccatgtcgatttccaggtgcaacgcaatccgaaaacggaaattctggaccagctgccgaaaaacaagatctcaaccgcaaaagtggattcgacgtttagttattccctgggcggtaaattcgacagcaccaaaggcattggtcgcaccagcagcaacagctactcgaagagcatctcttacaaccagcaaaactacgataccatcgcaagcggcaaaaacaataaccgtcatgttcactggtctgtggttgctaatgatctgaagtatggtaacgaaatcaaaaatcgcaacgacgaatttctgttctaccgtaatacccgcctgagtacggtcgaaaacccggaactgtcatttgcgtcgaaatatcgttacccggccctggttcgctccggctttaatccggaatttctgacctacatcagcaacgaaaagtctaacgaaaagacgcgtttcgaagtgacctatacgcgcaatcaggatatcctgaaaaacaagccgggcattcactacggtcagccgatcctggaacaaaacaaagatggccagcgttttattgtcgtgtatgaagtggactggaaaaataagaccgttaaggttgtcgaaaaatattctgatcagaacaagccgtacaaagaaggt

SEQ ID NO: 74 - protein, 322 aa, Staphylococcus aureus
ANKDSQDQTKKEHVDKAQQKEKRNVNDKDKNTPGPDDIGKNGKVTKRTVSEYDKETNILQNLQFDFIDDPTYDKNVLLVKKQGSIHSNLKFESHRNETNASWLKYPSEYHVDFQVQRNPKTEILDQLPKNKISTAKVDSTFSYSLGGKFDSTKGIGRTSSNSYSKSISYNQQNYDTIASGKNNNRHVHWSVVANDLKYGNEIKNRNDEFLFYRNTRLSTVENPELSFASKYRYPALVRSGFNPEFLTYISNEKSNEKTRFEVTYTRNQDILKNKPGIHYGQPILEQNKDGQRFIVVYEVDWKNKTVKVVEKYSDQNKPYKEG

SEQ ID NO: 75 - DNA, 945 bp, Staphylococcus aureus
gcaagctcgtatgcggaaatcaaaagcaagatcaccaccgtctcagaaaagaacctggatggcgacaccaagatgtacacccgtaccgcgaccacgagcgatacggaaaagaaaattagccagtctctgcaatttaatttcctgaccgaaccgaactacgacaaagaaacggtgtttattaaagccaagggcaccatcggcagcggtctgaaaattctgaatccgaacggctactggaacagcaccctgcgttggccgggtagttattccgtttcaattcagaacgtcgatgacaacaataactcaaccaatgtcacggattttgcaccgaaaaaccaagacgaatcgcgtgaagtgaaatatacctacggctataagacgggcggtgatttcagtatcaatcgcggcggtctgaccggtaacatcacgaaggaaaagaactactcggaaaccatcagctaccagcaaccgtcttatcgtaccctgattgatcagccgaccacgaataaaggcgtcgcgtggaaggtggaagcccatagcatcaataacatgggtcatgatcacacccgtcaactgacgaacgactctgatgaccgcgtgaaatctgaaatttttagtctgacccgcaatggcaacctgtgggcaaaagataatttcacgccgaaaaacaagatgccggtgaccgtttccgaaggctttaatccggaatttctggctgttatgtcccatgataaaaacgacaaaggtaagtcacgtttcatcgtccactataaacgctcgatggatgactttaaactggattggaataagcatggcttctggggttactggagtggtgaaaaccacgttgaccagaaagaagaaaagctgtccgccctgtatgaagtggattggaaaacgcacgacgttaaactgattaagaccatcaacgataaagaacagaag

SEQ ID NO: 76 - protein, 315 aa, Staphylococcus aureus
ASSYAEIKSKITTVSEKNLDGDTKMYTRTATTSDTEKKISQSLQFNFLTEPNYDKETVFIKAKGTIGSGLKILNPNGYWNSTLRWPGSYSVSIQNVDDNNNSTNVTDFAPKNQDESREVKYTYGYKTGGDFSINRGGLTGNITKEKNYSETISYQQPSYRTLIDQPTTNKGVAWKVEAHSINNMGHDHTRQLTNDSDDRVKSEIFSLTRNGNLWAKDNFTPKNKMPVTVSEGFNPEFLAVMSHDKNDKGKSRFIVHYKRSMDDFKLDWNKHGFWGYWSGENHVDQKEEKLSALYEVDWKTHDVKLIKTINDKEQK

SEQ ID NO: 77 - DNA, 954 bp, Staphylococcus aureus
gactcacaggaccaaaacaaaaaggaacacgttgataaggcacagcagaaagacaagcaagatagcaccaagaaaggcaaaaacgttgcggccccggatgacgtcggcaaaaacggcaaggtgaccaaacgtacggaaagcgaatacgatgaaaagaccaacatcctgcagaacctggaatttaatttcatcgatgacccgacctacgataaagacgtcctgctggtgaaaaagcaaggcagtattcattccaacctgaagttcgaaagtcacaaagaagaaaagaacagcacctggctgaaatatccgtcagaataccatgttgatttccaggtcaagcgtaacccgaaaaccgaaattctggaccaactgccgaaaaataagatcagtacggcaaaagtggattcaaccttttcgtatacgctgggcggtaaattcgactccattaaaggcatcggtcgcaatagctctaacagctattctcagaccatttcgtataatcagcaaaactacgatacgatcgcgagcggcaaaaacaataactggcatgtgcactggtctgttattgccaacgatctgaagtatggcggtgaagttaaaaatcgtaacgacgaatttctgttctaccgtaacacccgcacgagttccgttgataatccggaatcatcgtttgcagctaaatatcgttacccggcactggtccgcagtggttttaatccggaatttctgacctatctgagcaacgaaaagtctaatgaaaaaacgcagtttgaagtgacctatacgcgtaaccaagatatcctgaaaaatagcccgggcctgcattacgctccgccgattctggaaaagaacaaggttggtcaccgctttatcgtcacctatgaagtggattggaaaaataagacggtgaaggtggttgacaaatactctgatgaccagccgttccgcgaaggt

SEQ ID NO: 78 - protein, 318 aa, Staphylococcus aureus
DSQDQNKKEHVDKAQQKDKQDSTKKGKNVAAPDDVGKNGKVTKRTESEYDEKTNILQNLEFNFIDDPTYDKDVLLVKKQGSIHSNLKFESHKEEKNSTWLKYPSEYHVDFQVKRNPKTEILDQLPKNKISTAKVDSTFSYTLGGKFDSIKGIGRNSSNSYSQTISYNQQNYDTIASGKNNNWHVHWSVIANDLKYGGEVKNRNDEFLFYRNTRTSSVDNPESSFAAKYRYPALVRSGFNPEFLTYLSNEKSNEKTQFEVTYTRNQDILKNSPGLHYAPPILEKNKVGHRFIVTYEVDWKNKTVKVVDKYSDDQPFREG

SEQ ID NO: 79 - DNA, 927 bp, Staphylococcus aureus
aaaatcaaatcggaaatcacgcaagttagcgaacagaatatcgacggcaatacgaagatgtttacccgcacggcaacgacctcggatagccagaaaaagatcagccagtctctgcaatttaacttcctgaccgaaccgaactacgacaaggaaacggtgttcatcaaggcaaagggcaccatcggctctggtctgaaaattctggacccgaacggctactggaatagtaccctgcgttggccgggtagttattccgtgtcaatccagaacgttgataacaataccaatacgaaggttacggattttgccccgaaaaaccaagacgaaacccgcgaagtcaagtatacctacggctataaaacgggcggtgatttctcgattagcccgggcggtattaccggtaacatcacgaaagaacgtaattattctgaaaccatcagttaccagcaaccgagttatcgcaccctgattgaccagccggcgacgaataagggcgttggttggaaagtcgaagcccatctgatcaacaatatgggccatgatcacacccgtcaactgacgaacgattccgacaatcgcgtgggctcagaaatttttaccctgacgcgtaacggtaatctgtgggcgaaagataacttcacgccgaaaaataagatgccggtcaccgtgtccgaaggctttaacccggaatttctggccgttatgtcgcatgataaaaaggacaaaggcaagagcaaatttgtggttcactataaacgtacgatggatgactttaaaatcgattggatgcgccatggcttctggggttactggaccggtaaaaatcacgttgaccagaaggaagaaaaactgtctgcactgtatgaagtcgattggaaaacccacgacgtgaagttcattaaagctctggatgacaaagaaaagaaa

SEQ ID NO: 80 - protein, 309 aa, Staphylococcus aureus
KIKSEITQVSEQNIDGNTKMFTRTATTSDSQKKISQSLQFNFLTEPNYDKETVFIKAKGTIGSGLKILDPNGYWNSTLRWPGSYSVSIQNVDNNTNTKVTDFAPKNQDETREVKYTYGYKTGGDFSISPGGITGNITKERNYSETISYQQPSYRTLIDQPATNKGVGWKVEAHLINNMGHDHTRQLTNDSDNRVGSEIFTLTRNGNLWAKDNFTPKNKMPVTVSEGFNPEFLAVMSHDKKDKGKSKFVVHYKRTMDDFKIDWMRHGFWGYWTGKNHVDQKEEKLSALYEVDWKTHDVKFIKALDDKEKK